(19) United States
(12) Patent Application Publication
(10) Pub. No.: US 2025/0295758 A1
(43) Pub. Date: Sep. 25, 2025

(54) NOVEL PEPTIDE CONJUGATE VACCINES

(71) Applicant: STICHTING AMSTERDAM UMC, Amsterdam (NL)

(72) Inventors: Arjan Willem GRIFFIOEN, Amsterdam (NL); Elisabeth Johanna Maria HUIJBERS, Amsterdam (NL); Karlijn VAN LOON, Amsterdam (NL)

(21) Appl. No.: 18/860,476

(22) PCT Filed: Apr. 28, 2023
(86) PCT No.: PCT/NL2023/050226

§ 371 Date: Oct. 25, 2024

(30) Foreign Application Priority Data
Apr. 28, 2022 (NL) 2031731

(51) Int. Cl. A61K 39/215; A61K 39/00; A61P 37/04; C07K 14/005; C07K 14/245

(52) U.S. Cl. CPC A61K 39/215; A61P 37/04; C07K 14/005; C07K 14/245; A61K 2039/6031; C07K 2319/00; C12N 2770/20022; C12N 2770/20034; C12N 2770/20071

(57) ABSTRACT

The present invention provides novel fusion polypeptides and compositions comprising the same. The fusion polypeptides are able to trigger and/or enhance an immune response against a peptide sequence of interest comprised therein and therefore are useful for vaccinating or treating a mammalian subject. Kits comprising the fusion polypeptide are also provided.

The present invention provides novel fusion polypeptides and compositions comprising the same. The fusion polypeptides are able to trigger and/or enhance an immune response against a peptide sequence of interest comprised therein and therefore are useful for vaccinating or treating a mammalian subject. Kits comprising the fusion polypeptide are also provided.

### BACKGROUND

The rapid spread of infectious diseases across the globe as well as the increasing number of cancerous diseases demonstrates the increasing number of challenges for the immune system of each individual. In cases of infectious disease, the immune system of a mammal is triggered to fight antigens that are non-mammalian, i.e., that are non-self. In contrast thereto, in cases of cancer, eliciting a specific immune response against certain self-antigens is desirable.

Poor immunogenicity of critical disease-associated epitopes is one of the major drawbacks for protein-based vaccine development and efficacy against infectious diseases. Even for disease-associated epitopes with high immunogenicity, it can be desirable to boost the immune response to obtain a stronger humoral and/or cellular immune response. In both of these circumstances, there is a need to boost the immune response to a non-self-antigen.

By contrast, when protein-based therapeutics are used in the treatment of cancer, it is essential that they elicit an immune response to target tumor-specific self-antigens. Historically, it has been extremely difficult to generate a humoral and/or cellular immune response against such self-antigens. This difficulty results from immunological tolerance mechanisms that prevent the antigen-driven expansion of B cells and/or T cells with self-specificities. For these reasons, developing protein-based vaccines for use in treating cancer is a challenge. In such circumstances, there is a need to boost the immune response to a self-antigen.

There is a need for novel compositions that induce a clinically relevant immune response to a self-antigen or a non-self-antigen in a mammal.

### BRIEF SUMMARY OF THE DISCLOSURE

The inventors have studied the potential use of immune stimulating peptide sequences in order to trigger and/or enhance immune responses against non-mammalian peptide sequences or mammalian peptide sequences. When the immune system encounters an antigen which is recognized as being non-self, i.e. foreign, an immune response will typically be initiated against this antigen. Some foreign antigens will trigger a strong immune response whereas others will trigger only a weak or even no immune response at all. In some cases, it can be desirable to enhance the immune response to non-self antigens further.

The inventors have suprisingly observed that when immunizing a mammalian subject with a fusion polypeptide comprising a non-mammalian antigen of interest together with an immune stimulating peptide, a faster, stronger and more balanced immune response against the antigen of interest can be obtained compared to immunization with the antigen of interest alone. Suprisingly, an increased immune response is observed even when the non-self-antigen of interest is an antigen that would be expected to elicit an immune response when used on its own (e.g. a viral antigen). This advantage is considered of utmost importance during the development of a protein-based therapeutic against highly infectious agents. The immune stimulating peptides described herein may therefore advantageously be used as part of a fusion polypeptide that comprises a non-self-antigen of interest, in order to boost the immune response against the non-self-antigen.

The immune stimulating peptides provided herein have also been found to be suprisingly useful when linked to a self-antigen of interest. The inventors have surprisingly found that by administering a “self-antigen” linked to the specific immune stimulating peptide sequences described herein, a clinically relevant immune response can be triggered against the self-antigen. This is particularly useful e.g. when the self-antigen is a disease-associated antigen, such as a self-antigen that is associated with cancer.

Several different immune stimulating peptide sequences are described herein, for example SEQ ID NO: 5 (also referred to herein as “CDP” or “chimeric designer peptide”); SEQ ID NO: 6 (also referred to herein as “IDP1” or “iBoost designer partner 1”); SEQ ID NO: 7 (also referred to herein as “IDP2” or “iBoost designer partner 2”); SEQ ID NO: 8 (also referred to herein as “IDP3” or “iBoost designer partner 3”); SEQ ID NO: 10 (also referred to herein as “TRXtrunc”); SEQ ID NO: 11 (also referred to herein as “Type-1 fimbrial protein, A”); and SEQ ID NO: 12 (also referred to herein as “Variant Type-1 fimbrial protein, A”).

The inventors considered several different criteria when designing the immune stimulating peptide sequences described herein, and they found that the ratio of bulky hydrophilic or charged amino acids to the total amino acids within the immune stimulating peptide sequence provided a reliable measure of the efficacy of the immune stimulating peptide sequence in stimulating an immune response to a linked antigen of interest. Additional criteria that they considered to further optimize the immune stimulating peptide sequence per se include optimization of its length, isoelectric point (pI), immunogenicity (e.g. the presence of predicted T cell epitopes) and/or solubility. It is well understood that some of the criteria are associated with each other and by considering one another might change as well. For example, by increasing the solubility, immunogenicity may also be increased.

The inventors also considered these factors in respect of the fusion polypeptide as a whole (i.e. comprising an immune stimulating peptide sequence as described herein linked to an antigen sequence of interest). For example, low pI, high immunogenicity, optimal size of the fusion polypeptide, and/or high solubility may be desirable characteristics. The choice of immune stimulating peptide sequence may therefore be based on its desired effect on these characteristics of the fusion polypeptide as a whole. Based on these considerations, the optimal fusion polypeptide for therapeutic use may be generated.

The inventors found that when considering these criteria in their design of an immune stimulating peptide sequence (wherein the designed immune stimulating peptide sequence fulfills at least some or all of these criteria), an immune response against almost any peptide sequence of interest (irrespective of whether it is self, non-self, immunogenic, or non-immunogenic) can be elicited and/or enhanced. Whereas for certain antigens of interest it might be sufficient to only factor in one of these criteria, other antigens might require the consideration of two or more criteria together. Thus, for some antigens it might be sufficient if the ratio of bulky hydrophilic or charged amino acids to the total amino acids within the immune stimulating peptide is set properly. By contrast, for other antigens, immune stimulating peptide sequences that also lower the overall pI of the fusion polypeptide, increase immunogenicity of the antigen of interest, and/or increase the overall solubility fusion polypeptide may be desirable.

In one aspect, the invention provides a fusion polypeptide for use in vaccinating or treating a mammalian subject, the fusion polypeptide comprising:
(i) a non-mammalian peptide sequence, wherein the peptide sequence is a sequence that is associated with a disease or disorder against which the mammalian subject is being vaccinated or treated; and
(ii) an immune stimulating peptide sequence having a ratio of bulky hydrophilic or charged amino acids to total amino acids of less than or equal to 0.55.

Suitably, the fusion polypeptide may have an isoelectric point (pI) of less than or equal to 6.

Suitably, the non-mammalian peptide sequence may be a viral peptide and the disease or disorder may be a viral infection.

Suitably, the non-mammalian peptide sequence may be a mycobacterial or a bacterial peptide and the disease or disorder may be a mycobacterial or a bacterial infection.

Suitably, the non-mammalian peptide sequence may be a yeast peptide and the disease or disorder may be a yeast infection.

Suitably, the non-mammalian peptide sequence may be a parasite peptide and the disease or disorder may be a parasite infection.

Suitably, the non-mammalian peptide sequence may be a SARS-CoV-2 peptide and the disease or disorder may be a SARS-CoV-2 infection.

Suitably, the SARS-CoV-2 peptide may be a SARS-CoV-2 receptor binding domain (RBD) peptide.

Suitably, the RBD peptide may comprise the amino acid sequence: NITNLCPFGEVFNATRFASVYAWNRKRISNCVADYSVLYNSASFSTFKCYGVSPTKLNDLCFTN VYADSFVIRGDEVRQIAPGQTGKIADYNYKLPDDFTGCVIAWNSNNLDSKVGGNYNYLYRLFRK SNLKPFERDISTEIYQAGSTPCNGVEGFNCYFPLQSYGFQPTNGVGYQPYRVVVLSFELLHAP ATV (SEQ ID NO:1), or a conservative amino acid variant thereof.

Suitably, SARS-CoV-2 peptide is a S protein peptide, optionally wherein:
(a) the S protein peptide is a S1 subdomain peptide, further optionally wherein the S1 subdomain peptide is a RBD peptide (such as a receptor binding motif (RBM) peptide), a SD1 peptide or a SD2 peptide, or a combination thereof;
(b) the S protein peptide is a S2 subdomain peptide, further optionally wherein the S2 subdomain peptide is a S1/S2 peptide, a FP+S2′ peptide, or a HR1 peptide, or a combination thereof.

Suitably, the SARS-CoV-2 peptide comprises the amino acid sequence:
(a) of SEQ ID NO:39, or a conservative amino acid variant thereof;
(b) of SEQ ID NO:40, or a conservative amino acid variant thereof; or
(c) of SEQ ID NO:41, or a conservative amino acid variant thereof, preferably wherein the immune stimulating peptide comprises the amino acid sequence of SEQ ID NO:5.

Suitably, the non-mammalian peptide sequence may be an influenza virus peptide and the disease or disorder may be influenza.

Suitably, the influenza virus peptide sequence is a H1 influenza virus peptide, a N1 influenza virus peptide, or a H1/N1 influenza virus peptide.

Suitably, the influenza virus peptide comprises the amino acid sequence:
(a) of SEQ ID NO:42, or a conservative amino acid variant thereof;
(b) of SEQ ID NO:43, or a conservative amino acid variant thereof; or
(c) of SEQ ID NO:44, or a conservative amino acid variant thereof, preferably wherein the immune stimulating peptide comprises the amino acid sequence of SEQ ID NO:5.

Suitably, the immune stimulating peptide sequence may be at least 40 amino acids long and no more than 150 amino acids long.

Suitably, the immune stimulating peptide sequence may have an isoelectric point (pI) of less than or equal to 4.

Suitably, the immune stimulating peptide sequence may comprise the amino acid sequence DDEDFVDEDDD (SEQ ID NO:4) of the RRAB protein or a conservative amino acid variant thereof.

Suitably, the immune stimulating peptide sequence may comprise at least one predicted T cell epitope.

Suitably, the predicted T cell epitopes may be selected from: a MSYB protein T cell epitope and/or a RRAB protein T cell epitope.

Suitably, the MSYB protein T cell epitope may be comprised within the amino sequence: NPGIDAEDANVQQFNAQKYVLQDGDIMWQV (SEQ ID NO:2), and/or EGEFQLEPPLDTEEGRAAADE (SEQ ID NO:3), or a conservative amino acid variant thereof.

Suitably, the immune stimulating peptide sequence may comprise the amino acid sequence:
a) DNNSLSQEVQNGSNHLENNQSQSNGGGSDSALSLSSKTAALAAATTVNDGSDGA TSSAVG (SEQ ID NO:5), or a conservative amino acid variant thereof; or
b) HHAHTMYATLEEAIDAAREEFLADNPGIDAEDANVQQFNAQKYVLQDGDIMWQVE FFADEGEEGEDDEDFVDEDDD (SEQ ID NO:6), or a conservative amino acid variant thereof; or
c) HHAHNPGIDAEDANVQQFNAQKYVLQDGDIMWQVEGEFQLEPPLDTEEGRAAAD EDDEDFVDEDDD (SEQ ID NO:7), or a conservative amino acid variant thereof; or
d) HHAHNPGIDAEDANVQQFNAQKYVLQDGDIMWQVDDEDFVDEDDD (SEQ ID NO:8), or a conservative amino acid variant thereof.

In one aspect, the invention provides a fusion polypeptide for use in vaccinating or treating a mammalian subject, the fusion polypeptide comprising:
(i) a non-mammalian peptide sequence, wherein the peptide sequence is a sequence that is associated with a disease or disorder against which the mammalian subject is being vaccinated or treated; and
(ii) an immune stimulating peptide sequence comprising the amino acid sequence:
a) DNNSLSQEVQNGSNHLENNQSQSNGGGSDSALSLSSKTAALAAATTVNDGSDGA TSSAVG (SEQ ID NO:5), or a conservative amino acid variant thereof; or
b) HHAHTMYATLEEAIDAAREEFLADNPGIDAEDANVQQFNAQKYVLQDGDIMWQVE FFADEGEEGEDDEDFVDEDDD (SEQ ID NO:6), or a conservative amino acid variant thereof; or
c) HHAHNPGIDAEDANVQQFNAQKYVLQDGDIMWQVEGEFQLEPPLDTEEGRAAAD EDDEDFVDEDDD (SEQ ID NO:7), or a conservative amino acid variant thereof; or
d) HHAHNPGIDAEDANVQQFNAQKYVLQDGDIMWQVDDEDFVDEDDD (SEQ ID NO:8), or a conservative amino acid variant thereof.

In another aspect, the invention provides a fusion polypeptide comprising:
(i) a non-mammalian peptide sequence, wherein the peptide sequence is a sequence that is associated with a disease or disorder against which the mammalian subject is being vaccinated or treated; and
(ii) an immune stimulating peptide sequence having a ratio of bulky hydrophilic or charged amino acids to total amino acids of less than or equal to 0.55;
   wherein the non-mammalian peptide sequence is linked to the immune stimulating peptide sequence and wherein the immune stimulating peptide sequence comprises the amino acid sequence:
a) HHAHTMYATLEEAIDAAREEFLADNPGIDAEDANVQQFNAQKYVLQDGDIMWQVE FFADEGEEGEDDEDFVDEDDD (SEQ ID NO:6), or a conservative amino acid variant thereof; or
b) HHAHNPGIDAEDANVQQFNAQKYVLQDGDIMWQVEGEFQLEPPLDTEEGRAAAD EDDEDFVDEDDD (SEQ ID NO:7), or a conservative amino acid variant thereof; or
c) HHAHNPGIDAEDANVQQFNAQKYVLQDGDIMWQVDDEDFVDEDDD (SEQ ID NO:8), or a conservative amino acid variant thereof; or
d) DNNSLSQEVQNGSNHLENNQSQSNGGGSDSALSLSSKTAALAAATTVNDGSDGA TSSAVG (SEQ ID NO:5), or a conservative amino acid variant thereof.

In another aspect, the invention provides a fusion polypeptide comprising:
(i) a non-mammalian peptide sequence, wherein the peptide sequence is a sequence that is associated with a disease or disorder against which the mammalian subject is being vaccinated or treated; and
(ii) an immune stimulating peptide sequence;
   wherein the non-mammalian peptide sequence is linked to the immune stimulating peptide sequence and wherein the immune stimulating peptide sequence comprises the amino acid sequence:
a) HHAHTMYATLEEAIDAAREEFLADNPGIDAEDANVQQFNAQKYVLQDGDIMWQVE FFADEGEEGEDDEDFVDEDDD (SEQ ID NO:6), or a conservative amino acid variant thereof; or
b) HHAHNPGIDAEDANVQQFNAQKYVLQDGDIMWQVEGEFQLEPPLDTEEGRAAAD EDDEDFVDEDDD (SEQ ID NO:7), or a conservative amino acid variant thereof; or
c) HHAHNPGIDAEDANVQQFNAQKYVLQDGDIMWQVDDEDFVDEDDD (SEQ ID NO:8), or a conservative amino acid variant thereof; or
d) DNNSLSQEVQNGSNHLENNQSQSNGGGSDSALSLSSKTAALAAATTVNDGSDGA TSSAVG (SEQ ID NO:5), or a conservative amino acid variant thereof.

In another aspect, the invention provides a fusion polypeptide comprising:
(i) a mammalian peptide sequence, wherein the peptide sequence is a sequence that is associated with a disease or disorder against which the mammalian subject is being vaccinated or treated; and
(ii) an immune stimulating peptide sequence having a ratio of bulky hydrophilic or charged amino acids to total amino acids of less than or equal to 0.55;
   wherein the mammalian peptide sequence is linked to the immune stimulating peptide sequence and wherein the immune stimulating peptide sequence comprises the amino acid sequence:
a) HHAHTMYATLEEAIDAAREEFLADNPGIDAEDANVQQFNAQKYVLQDGDIMWQVE FFADEGEEGEDDEDFVDEDDD (SEQ ID NO:6), or a conservative amino acid variant thereof; or
b) HHAHNPGIDAEDANVQQFNAQKYVLQDGDIMWQVEGEFQLEPPLDTEEGRAAAD EDDEDFVDEDDD (SEQ ID NO:7), or a conservative amino acid variant thereof; or
c) HHAHNPGIDAEDANVQQFNAQKYVLQDGDIMWQVDDEDFVDEDDD (SEQ ID NO:8), or a conservative amino acid variant thereof.

In another aspect, the invention provides a fusion polypeptide comprising:
(i) a mammalian peptide sequence, wherein the peptide sequence is a sequence that is associated with a disease or disorder against which the mammalian subject is being vaccinated or treated; and
(ii) an immune stimulating peptide sequence;
   wherein the mammalian peptide sequence is linked to the immune stimulating peptide sequence and wherein the immune stimulating peptide sequence comprises the amino acid sequence:
a) HHAHTMYATLEEAIDAAREEFLADNPGIDAEDANVQQFNAQKYVLQDGDIMWQVE FFADEGEEGEDDEDFVDEDDD (SEQ ID NO:6), or a conservative amino acid variant thereof; or
b) HHAHNPGIDAEDANVQQFNAQKYVLQDGDIMWQVEGEFQLEPPLDTEEGRAAAD EDDEDFVDEDDD (SEQ ID NO:7), or a conservative amino acid variant thereof; or
c) HHAHNPGIDAEDANVQQFNAQKYVLQDGDIMWQVDDEDFVDEDDD (SEQ ID NO:8), or a conservative amino acid variant thereof.

Suitably, the fusion polypeptide may have an isoelectric point (pI) of less than or equal to 6.

Suitably, the non-mammalian peptide sequence may be a viral peptide and the disease or disorder may be a viral infection.

Suitably, the non-mammalian peptide sequence may be a mycobacterial or a bacterial peptide and the disease or disorder may be a mycobacterial or a bacterial infection.

Suitably, the non-mammalian peptide sequence may be a yeast peptide and the disease or disorder may be a yeast infection.

Suitably, the non-mammalian peptide sequence may be a parasite peptide and the disease or disorder may be a parasite infection.

Suitably, the mammalian peptide sequence may be associated with tumor angiogenesis.

Suitably, the mammalian peptide sequence may be vimentin (Vim), apelin, notum or timp1.

Suitably, the non-mammalian peptide sequence may be a SARS-CoV-2 peptide.

Suitably, the SARS-CoV-2 peptide may be a SARS-CoV-2 receptor binding domain (RBD) peptide.

Suitably, the RBD peptide may comprise the amino acid sequence: NITNLCPFGEVFNATRFASVYAWNRKRISNCVADYSVLYNSASFSTFKCYGVSPTKLNDLCFTN VYADSFVIRGDEVRQIAPGQTGKIADYNYKLPDDFTGCVIAWNSNNLDSKVGGNYNYLYRLFRK SNLKPFERDISTEIYQAGSTPCNGVEGFNCYFPLQSYGFQPTNGVGYQPYRVVVLSFELLHAP ATV (SEQ ID NO:1), or a conservative amino acid variant thereof.

Suitably, the SARS-CoV-2 peptide is a S protein peptide, optionally wherein:
(a) the S protein peptide is a S1 subdomain peptide, further optionally wherein the S1 subdomain peptide is a RBD peptide (such as a receptor binding motif (RBM) peptide), a SD1 peptide or a SD2 peptide, or a combination thereof;
(b) the S protein peptide is a S2 subdomain peptide, further optionally wherein the S2 subdomain peptide is a S1/S2 peptide, a FP+S2′ peptide, or a HR1 peptide, or a combination thereof.

Suitably, the SARS-CoV-2 peptide comprises the amino acid sequence:
(a) of SEQ ID NO:39, or a conservative amino acid variant thereof;
(b) of SEQ ID NO:40, or a conservative amino acid variant thereof; or
(c) of SEQ ID NO:41, or a conservative amino acid variant thereof.

Suitably, the non-mammalian peptide sequence is an influenza virus peptide sequence and the disease or disorder is influenza.

Suitably, the influenza virus peptide sequence is a H1 influenza virus peptide, a N1 influenza virus peptide, or a H1/N1 influenza virus peptide.

Suitably, the influenza virus peptide comprises the amino acid sequence:
(a) of SEQ ID NO:42, or a conservative amino acid variant thereof;
(b) of SEQ ID NO:43, or a conservative amino acid variant thereof; or
(c) of SEQ ID NO:44, or a conservative amino acid variant thereof.

Suitably, the immune stimulating peptide sequence may be at least 40 amino acids long and no more than 150 amino acids long. Suitably, the immune stimulating peptide sequence may be at least 40 amino acids long and no more than 100 amino acids long.

Suitably, the immune stimulating peptide sequence may have an isoelectric point (pI) of less than or equal to 4.

In another aspect, the invention provides a nucleic acid encoding a fusion polypeptide according to the invention, optionally wherein the nucleic acid is an expression vector.

In another aspect, the invention provides a pharmaceutical composition comprising a fusion polypeptide according to the invention, or a nucleic acid according to the invention, wherein the composition further comprises a pharmaceutically acceptable carrier, adjuvant, excipient or diluent.

In another aspect, the invention provides a fusion polypeptide according to the invention, or a composition according to the invention, for use as a medicament.

In another aspect, the invention provides a use of a fusion polypeptide according to the invention, or a composition according to the invention, for vaccination or treatment of a subject.

In another aspect, the invention provides a method for vaccinating or treating a subject, the method comprising administering a fusion polypeptide according to the invention, or a composition according to the invention, to the subject.

Various aspects of the invention are described in further detail below.

### BRIEF DESCRIPTION OF THE FIGURES

Embodiments of the invention are further described hereinafter with reference to the accompanying figures, in which “CDP” corresponds to SEQ ID NO:5:

FIG. 1 shows a conjugate vaccine targeting the RBD domain of SARS-CoV-2. (A-C) Schematic representation of the RBD domain (A), chimeric designer peptide (CDP) (B) and produced CDP-RBD (28.9 kDa) and RBD (22.8 kDa) vaccine proteins (C). (B) CDP is composed of several highly-immunogenic clusters originating from three different bacterial proteins (cell division protein ZapB, type-1 fimbrial protein TFP and small heat shock protein IbpA). (C) The theoretical isoelectric point (pI) and the predicted solubility score (Sol) for each vaccine protein. (D) Coomassie staining after SDS-PAGE (left) and anti-RBD western blot (right) analysis showing RBD and CDP-RBD. (E) Size exclusion chromatogram (10/300 GL Superdex 75, Cytivia) showing purified CDP-RBD (black arrow).

FIG. 2 shows the protein purification and CDP-RBD sequence. (A, B) Coomassie staining after SDS-PAGE analysis showing eluted fractions of RBD (A) or CDP-RBD (B) during purification using Ni-NTA beads. Proteins were eluted with four fractions of 200 mM imidazole, followed by four fractions elution buffer (pH 4.5, 8 M urea). (C) Amino acid sequence of the CDP-RBD protein. Predicted secondary structures and B cell as well as T cell epitopes are shown.

FIG. 3 shows a conjugate CDP-RBD vaccine elicits faster antibody responses compared to its unconjugated counterpart. (A) Timeline of mouse vaccinations. Mice were immunized with 100 μg of RBD or CDP-RBD at day 0 and at day 14. Sera were collected from all mice prior to vaccination (day 0) and at experimental days 13, 21, 28 and 35 (n=10 for days 13 and 21, n=5 for days 28 and 35). (B) Vaccine proteins were mixed with Montanide ISA 720 together with CpG 1826 oligonucleotide (MnC). (C) Anti-RBD total immunoglobulin (Ig) endpoint titers for both vaccine groups at experimental days 13, 21, 28 and 35 as assessed by ELISA. The number of responding mice is provided for each timepoint. (D) Anti-RBD total immunoglobulin (Ig) titers at day 21 (left) and day 35 (right). (E) Analysis of endpoint titers of RBD-specific IgG subclasses (IgG1, IgG2a, IgG2b, IgG3) at day 21. The number of responding mice is provided for each IgG subclass. (F) Surrogate virus neutralization assay. Circulating neutralizing antibodies in the sera of vaccinated mice inhibit the interaction between RBD and the ACE-2 receptor. (G, H) Surface Plasmon Resonance (SPR) biosensor assay. Binding of anti-RBD antibodies of mouse sera towards commercial RBD. Sera were diluted 1:100. Data are shown as geometric mean values ±geometric SD (C, E) or as mean values ±SD (D, F, H). Statistical significance was determined by an unpaired, Mann-Whitney test for each time point (C, H) or IgG subclass (E) or by a two-way ANOVA followed by Sidka's multiple-comparison test (F). (*p<0.05, **p<0.01).

FIG. 4 show the antibody responses upon vaccination with CDP-RBD. (A) Anti-RBD total immunoglobulin (Ig) levels for both vaccine groups at experimental days 0, 13, 21 and 35 were assessed by ELISA. Sera were diluted 1:100. (B) Analysis of anti-RBD IgG subclasses at day 21. Mouse sera were diluted 1:100. (C) Anti-CDP total Ig levels at day 21. Mouse sera were diluted 1:100. (D) Anti-RBD total Ig endpoint titers at day 21 after immunization with RBD or CDP-RBD in combination with the Sepivac adjuvant. (E) Effect of different vaccine dosages of CDP-RBD combined with either MnC or Sep on the RBD response. (F, G) Surface Plasmon Resonance (SPR) biosensor assay. Binding of anti-RBD antibodies of sera derived from mice immunized with RBD/Sep or CDP-RBD/Sep towards commercial RBD. Sera were diluted 1:100. Data are shown as mean values ±SD (A-D, G) or as geometric mean values ±geometric SD (E). Statistical significance was determined by a two-way ANOVA followed by Sidka's multiple-comparison test (A) or an unpaired, Mann-Whitney test for each subclass (B), vaccine group (C), CDP-RBD protein dosage (E) or time point (G). (ns; no significance, *p<0.05, ***p<0.001).

FIG. 5 shows that CDP-RBD vaccination evokes systemic T cell immunity and further improves the CTL response in mice compared to RBD. (A) Timeline and experimental procedure followed for the detection of splenic RBD-specific T cells in immunized mice. (B, D) Representative dot plots depicting IFNγ and TNFα expression in CD4+ T cells (B) and CD8+ T cells (D) upon no stimulation (−) or ex vivo stimulation (+) with a SARS-CoV-2 Peptide Mix for 5 hours in the presence of Brefeldin A. (C, E) Quantification of the percentage (%) of RBD-reactive IFNγ+(monofunctional) and IFNγ+TNFα+ (polyfunctional) CD4+ (C) and CD8+ (E) T cells upon vaccination with the RBD/MnC or CDP-RBD/MnC regimens (n=5 mice per group). Each dot represents the mean value of two technical replicates. Error bars indicate mean frequencies ±SD. Statistical significance was determined by an unpaired Student's t test (ns; no significance, *p<0.05). (F) Heatmap showing the correlation between different components of the humoral and cellular immune response induced in mice 21 days after immunization with RBD/MnC or CDP-RBD/MnC. The Y axis corresponds to antibody end titers (Total Ig, IgG1, IgG2a, IgG2b, IgG3) or the % of RBD:ACE-2 binding inhibition (nAbs), while the X axis corresponds to the % of RBD-specific T cells. The Pearson correlation coefficient (r) value was used for plotting and for estimating the relevance of the features included. (G, H) Representative scatter plots illustrating the correlation between the % of RBD-reactive CD4+ (G) or CD8+ (H) T cells and the corresponding Total Ig endpoint titers. Regression lines denote linear regression for each vaccine group. Both the Pearson correlation coefficient (r) and the coefficient of determination (r2) values are provided for each scatter plot.

FIG. 6 shows RBD-specific T cell responses induced upon vaccination with RBD or CDP-RBD. (A) Gating strategy applied during flow cytometric analysis. Gated CD4+ and CD8+ T cells were selected for further analysis. (B) Percentage (%) of IFNγ+, TNFα+, and IFNγ+TNFα+CD4+ or CD8+ T cells detected in the absence or presence of the SARS-CoV-2 Peptide Mix. Each dot represents the mean value of two technical replicates (n=5 mice per group).

FIG. 7 shows that CDP-RBD elicits neutralizing antibodies and protects against SARS-CoV-2-mediated lung lesions in hamsters. (A) Timeline of the vaccination strategy and SARS-CoV-2 challenge of hamsters. Animals were immunized with 100 μg of CDP-RBD protein in combination with MnC as adjuvant or with Tris-sucrose (control) at day 0 and day 21. Sera were collected from all hamsters at experimental days 0, 21, 42, 46 and 49. At day 42 all hamsters were challenged with SARS-CoV-2. Four days post infection (dpi) half of the animals per group (4 out of 8) were sacrificed, at day 49 (7 dpi) the four remaining animals were sacrificed. (B) Anti-RBD IgG levels at experimental days 0, 21, 42, 46 and 49 as assessed by ELISA. Sera were diluted 1:100. (C) Throat swaps were analyzed to measure the replication of competent SARS-CoV-2 the first four dpi. (D) Weight progression after infection with SARS-CoV-2. At day 49 only the remaining 4 hamsters were weighed. (E) Surrogate virus neutralization assay with sera of hamsters at experimental day 0, 21, 42, 46 and 49. (F) Hematoxylin and eosin (HE) stained images of infected hamster lungs seven dpi (left). Lungs of control hamsters show strong infiltration of immune cells (asterisk). Quantification of lung lesions four and seven dpi (right). Scale bar, 100 μm. Data are shown as mean values ±SD. Statistical significance was determined by and unpaired, Mann-Whitney test for each time point (A) or two-way ANOVA followed by Sidak's multiple-comparison test (C-F). (*p<0.05, **p<0.01, ****p<0.0001).

FIG. 8 shows antibody response and viral load after hamster vaccination. (A) Anti-RBD IgG end point titers at experimental days 21, 42, 46 and 49 assessed by ELISA. (B) Analysis of competent virus in samples of lung and nasal turbines at experimental day 46. (C) HE stained lungs of control animals after SARS-CoV-2 infection. Hyperplasia of bronchial epithelial cells (BEC, double-headed arrow, left) and strong infiltration of immune cells (right) were visible. Scale bars, 100 μm (10×) or 25 μm (40×). Data are shown as geometric mean values ±geometric SD (A) or mean values ±SD (B). Statistical significance was determined by an unpaired, Mann-Whitney test for each time point (A) or organ (B).

FIG. 9 shows IDP1 (SEQ ID NO:6) fused to mouse Vimentin (63 kDa). Most of the IDP1-mVim is eluted in the imidazole fractions (11-14). Buffer E fractions (E1-E4), supernatant after sonication of the bacterial pellet (sup).

FIG. 10 shows IDP2 (SEQ ID NO:7) fused to mouse Vimentin (62 kDa). Most of the IDP2-mVim is eluted in the imidazole fractions (11-14). Buffer E fractions (E1-E4), supernatant after sonication of the bacterial pellet (sup).

FIG. 11 shows IDP3 (SEQ ID NO:8) fused to mouse Vimentin (60 kDa). Most of the IDP3-mVim is eluted in the imidazole fractions (11-14). Buffer E fractions (E1-E4), supernatant after sonication of the bacterial pellet (sup).

FIG. 12 shows CDP (SEQ ID NO:5) fused to mouse Vimentin (61 kDa). CDP-mVim is eluted in the buffer E fractions (E1-E4). Pooled imidazole fractions (I), wash of the column (W), supernatant after sonication of the bacterial pellet (sup).

FIG. 13 shows TRXtr (SEQ ID NO:10) fused to mouse Vimentin (61 kDa). TRXtr-mVim is eluted in the buffer E fractions (E1-E4). Pooled imidazole fractions (I), supernatant after sonication of the bacterial pellet (sup).

FIG. 14 shows an SDS-PAGE gel of the eluted fractions during protein purification of TRXtr (SEQ ID NO: 10) and CDP (SEQ ID NO:5) each fused to mouse apelin. TRXtr-Apelin (16.2 kDa) could only be eluted in buffer containing 100 mM EDTA (F1-F4), whereas when mouse apelin was conjugated to CDP (CDP-Apelin (16.0 kDa)) the protein was eluted in buffer containing 200 mM imidazole.

FIG. 15 shows an SDS-page gel of the eluted fractions obtained during protein purification of CDP2.2 (also referred to as IDP2) (SEQ ID NO:7) fused to mouse apelin (16.70 kDa). A) SDS-PAGE gel CDP2.2-Apelin 16.7 kDa is eluted mainly in 12 and 13. B) 12 of the SDS-PAGE gel on Western Blot. S (supernatant). Conjugation of mouse Apelin to CDP2.2 (IDP2) drastically decreases the isoelectric point (pI) of the fusion protein construct and makes elution of the protein with 200 mM imidazole possible (11-14) (FIG. 15A). The protein is mainly eluted in the imidazole fractions, meaning that imidazole competes with histidine binding and that elution is not dependent on low pH (buffer E) and thus changing of charge of the protein.

FIG. 16 shows the purification of different mouse Timp1 constructs. (A) a schematic of the steps for protein production and purification. (B) SDS-PAGE of the pellet and supernatant following sonication for: mouse Timp1, and mouse Timp1 fused to each of TRXtr (SEQ ID NO:10), CDP (SEQ ID NO:5), CDP2.1 (also referred to as IDP1) (SEQ ID NO:6), CDP 2.2 (also referred to as IDP2) (SEQ ID NO:7) and CDP2.3 (also referred to as IDP3) (SEQ ID NO:8)—thick bands at the size of the fusion proteins were found in the supernatant (white arrows). (C) Results from the purification of 5 mL supernatant with 200 μL of Ni-Agarose beads. TRXtr-Timp1 could only be eluted in buffer E, by changing of charge of the protein, whereas the CDP conjugated proteins can be eluted in imidazole.

FIG. 17 shows an SDS-page gel of the eluted fractions during protein purification of CDP (SEQ ID NO:5) fused to mouse Notum (62.0 kDa) eluted with 200 mM of imidazole (12 and 13).

FIG. 18 shows an SDS-page gel of the eluted fractions during protein purification of TRXtr (SEQ ID NO:10) fused to mouse Notum (62.3 kDa). TRXtr-Notum is eluted in the imidazole and the buffer E fractions.

FIG. 19 shows an SDS-page gel of the eluted fractions during protein purification of CDP 2.2 (SEQ ID NO:7) fused to mouse Notum (62.9 kDa). CDP2.2-Notum is mainly eluted in the imidazole fractions.

FIG. 20 shows an SDS-page gel of the eluted fractions during protein purification of CDP 2.3 (SEQ ID NO:8) fused to mouse Notum (60.5 kDa). CDP2.3-Notum is mainly eluted in the imidazole fractions.

FIG. 21 shows an SDS-page gel of the eluted fractions during protein purification of CDP (SEQ ID NO:5) fused to influenza protein sequences. Addition of CDP to the H1/N1 sequences lowers the isoelectric point of the protein and the protein can be eluted with imidazole (11-14).

FIG. 22 shows an SDS-page gel of the eluted fractions during protein purification of control influenza constructs. Influenza constructs not conjugated to CDP can only be eluted with buffer E (E1-E4).

FIG. 23 shows a schematic of immunization studies that have been performed in mice. Left: SDS-PAGE gel showing the different influenza constructs. Balb/c mice were vaccinated with 30 ug protein mixed with Sepivac adjuvant. A primer vaccination was given at day 0 and a booster at day 14. Blood samples were taken at day 0, day 7, day 21, day 28 and day 35 at end of the experiment.

FIG. 24 shows levels of Anti-H1 and anti-N1 antibodies in the sera of vaccinated mice. Conjugation of CDP to H1, N1 or H1N1 enhances the antibody response towards H1 (FIG. 24 A, circles), N1 (FIG. 24B, diamonds) and against H1N1 (FIGS. 24A and B; downward pointing triangles) in mice. The total amount of antibodies induced after vaccination with the CDP-H1N1 protein is equal to the amount of antibodies induced with the single constructs (CDP-H1, CDP-N1) (FIG. 25 below).

FIG. 25 shows levels of total anti-H1N1 antibodies in the sera of vaccinated mice.

FIG. 26 shows antibody titers induced with the different influenza constructs. Conjugation to CDP enhances the anti-H1, N1 and H1N1 antibody response in mice.

FIG. 27 shows (A) a schematic of the SARS-CoV2 virus, (B) a schematic of various functional domains of SARS-CoV2, namely, RBM, SD1, SD2, S1/S2 cleavage site, FP and S2′ protease cleavage site (FP+S2′) and HR1, (C) an outline of experiments involving BALB/c mice subcutaneously immunized with 100 μg CDP-complete vaccine protein mixed 1:1 (v/v) with Sepivac, an oil-in-water adjuvant—injections were given at day 1 and 14 and blood samples were taken at time points (0, 7, 13, 21 28 and 35 days post immunization), and (D) the corresponding antibody titers of each time point obtained using ELISA.

FIG. 28 shows (A) Colloidal Coomassie blue-stained SDS-PAGE gels of CDP-Complete, CDP-Binding and CDP-Fusion vaccines expressed in E. coli BL21 cells, and the OD levels (655 nm) of (B) anti S1/S2 antibody and (C) anti-RMB, anti-SD1, anti-SD2, anti-S1/S2, anti-FP, and anti-HR1 total Ig antibodies for each vaccine.

FIG. 29 shows (A) Spleens from mice immunized with CDP-Complete were isolated on day 35 and splenocytes restimulated ex vivo for 24 h with the RBM, SD1, SD2, S1/S2, FP+S2′ and HR1 synthesized peptides. Unstimulated splenocytes treated with Brefeldin A were taken along as a negative control. After stimulation for 5 hours in the presence of Brefeldin A, the frequency of antigen-specific CD4⁺ and CD8⁺ T cells was examined using flow cytometry. (B) A clear population of monofunctional (IFNγ⁺ or TNFα⁺), as well as multifunctional (IFNγ⁺ TNFα⁺) CD4⁺ T cells was observed after stimulation with each of the individual peptides. Indicating that systemic T cell immunity and CD8⁺ T cell responses are induced upon vaccination with the CDP-Complete vaccine.

FIG. 30 shows (A) body weight of the hamsters monitored during the experiment. Group 1: PBS control (circles), Group 2: CDP-Complete vaccine (squares) (B) Hamster body weight post SARS-CoV2 challenge (day 42-Day 49). (C) Virus load in lung and nasal turbinate samples at D+3 post infection. Data shown as mean of bacterial load ±SEM. Circles: PBS control, squares: CDP-Complete vaccine. (D) virus neutralization of the hamster sera DO-D7 post 1^{st} vaccination. circles: PBS control (n=8), squares: CDP-Complete vaccine (n=8). Upon SARS-CoV2 infection, animals immunized with the CDP-Complete vaccine developed a neutralizing antibody response earlier than the control group.

### DETAILED DESCRIPTION

The present invention provides novel fusion polypeptides and compositions comprising the same. The fusion polypeptides are able to trigger and/or enhance an immune response against a peptide sequence of interest comprised therein and therefore are useful for vaccinating or treating a mammalian subject. Kits comprising the fusion polypeptide are also provided.

### Vaccination and Treatment of a Subject

A fusion polypeptide for use in vaccinating or treating a mammalian subject is provided herein, the fusion polypeptide comprising:
(i) a non-mammalian peptide sequence, wherein the peptide sequence is a sequence that is associated with a disease or disorder against which the mammalian subject is being vaccinated or treated; and
(ii) an immune stimulating peptide sequence having a ratio of bulky hydrophilic or charged amino acids to total amino acids of less than or equal to 0.55.

The term “fusion polypeptide” as used herein refers to a polypeptide sequence comprising at least the two parts/polypeptide moieties of which one part is the sequence against which an immune response should be elicited/enhanced (antigen/epitope of interest) and wherein the other part, the immune stimulating peptide sequence, is the sequence that triggers and/or enhances the immune response against the antigen/epitope of interest. The two parts are typically in their native, folded conformation and are generally joined (“linked”) by their respective carboxyl and amino termini.

The terms “polypeptide”, “protein” and “peptide” encompass (poly)peptide analogs, which may have, for example, modifications rendering them more stable (e.g. in vivo) or more capable of penetrating into cells (e.g. in vivo). Such modifications include, but are not limited to, N-terminus modification, C-terminus modification, peptide bond modification, including, but not limited to, CH₂—NH, CH₂—S, CH₂—S=0, 0=C—NH, CH₂—O, CH₂—CH₂, S═C—NH, CH═CH or CF═CH and/or backbone modifications. Methods for preparing peptidomimetic compounds are well known in the art and are specified, for example, in Quantitative Drug Design, C. A. Ramsden Gd., Chapter 17.2, F. Choplin, Pergamon Press (1992).

There may be a peptide linker between the two parts of the fusion polypeptide (i.e. between the antigen/epitope of interest and the immune stimulating peptide sequence).

The terms “linker peptide”, “linker”, or “peptide linker”, as used herein, refer to amino acid sequences that connect or link at least two polypeptide moieties. The linker can be a tandem repeat linker. The tandem repeat linker may be a tandem repeat of the amino acids glycine-serine. For example, the linker may have the amino acid sequence GS or GSGS, or GSGSGS (SEQ ID NO:27).

In addition to the linker, the fusion polypeptide can further comprise a N-terminal or C-terminal tag, e.g. a N-terminal or C-terminal purification tag. For example, the purification tag can be a tag comprising the amino acid sequence HHAH or HHHHHH (His tag; SEQ ID NO:26).

The fusion polypeptide described herein (comprising an immune stimulating peptide sequence and an antigen/epitope of interest, optionally together with a linker peptide and/or a N-terminal or C-terminal tag) may have a predicted isoelectric point (pI) of less than or equal to 6.

The isoelectric point (pI) of a polypeptide is directly correlated to its solubility and has a direct impact on the ability of the fusion polypeptide to trigger or enhance an immune response against the antigen of interest.

The “isoelectric point” (pI) as used herein is the pH of a solution at which the net charge of a protein becomes zero. At solution pH that is above the pI, the surface of the protein is predominantly negatively charged. Proteins are expected to be 50% soluble near their isoelectric points. Fusion polypeptides as described herein with a pI that is less than physiological pH (of around 7.4) will have increased solubility in vivo since the overall charge of the protein is negative, which is desirable when eliciting an immune response, as precipitated/insoluble protein will not elicit an immune response. One of the explanations for this is that only particles of a certain size can be taken up by antigen presenting cells. Proteins with a pI above physiological pH are not soluble because they are positively charged.

Methods for determining the predicted pI of a protein are well known in the art (for example https://web.expasy.org/protparam/).

In one example, the predicted pI of the fusion polypeptide described herein is less than or equal to 5.5 or less than or equal to 5.

In another example, the predicted pI of the fusion polypeptide described herein is less than or equal to 4.5, or less than or equal to 4.

By providing a fusion polypeptide as described herein, it is possible to elicit an immune response against an antigen of interest, irrespective of whether the antigen itself has a high, medium, low or even no immunogenicity. An antigen with low or no immunogenicity would not activate the immune system of a subject or would only do so to a very small extent which might not be sufficient to provide protection for the subject against the pathogen from which the antigen originates. Thus, with a fusion polypeptide as described herein, the mentioned drawbacks for vaccine development and efficacy due to a too weak immune response of some epitopes can be overcome. With a fusion polypeptide as described herein it is therefore possible to boost the immunogenicity of an antigen of interest (e.g. a non-self antigen or a self antigen).

The fusion polypeptides provided herein are for administration to a mammalian subject. The fusion polypeptides may be used to elicit or boost an immune response in a mammalian subject against an antigen of interest, wherein the antigen of interest may be a “self antigen” or a “non-self antigen”. A person of skill in the art will readily be able to identify self antigens and non-self antigens by reference to the subject to which the fusion polypeptide is being administered. For example, when the mammalian subject is a human, “self antigens” correspond to antigens that originate/are present in human proteins. The term “self antigen” encompasses “neo antigens”, which are self antigens but which are recognized as foreign antigens by the immune system. Similarly, when the mammalian subject is a human, “non-self antigens” correspond to antigens that do not originate/are not present in human proteins. In another example, for any mammalian subject, peptide sequences that do not originate from/are not present in mammals (non-mammalian peptide sequences) may be referred to as “non-self antigens”.

In some examples, the fusion polypeptides described herein comprise a non-mammalian peptide sequence, wherein the non-mammalian peptide sequence is a sequence that is associated with a disease or disorder against which the mammalian subject is being vaccinated or treated. In this context the non-mammalian peptide sequence may also be described as a peptide sequence comprising a non-mammalian antigen/epitope of interest. In other words, it may be described as a peptide sequence comprising a non-self antigen/epitope of interest. Alternatively, it may be described as a peptide sequence comprising a foreign antigen/epitope of interest. The terms “non self” and “foreign” are used interchangeably herein.

The non-mammalian peptide sequence may originate from a pathogen that is associated with a disease or disorder. In this context, the term “associated with” means that the pathogen, in particular certain specific parts of the pathogen, are responsible for the onset, outbreak, worsening or relapse of a disease appearing when an infection with this pathogen takes place. This also includes secondary diseases based on an infection by such a pathogen.

In one example, the non-mammalian peptide sequence is a mycobacterial or a bacterial peptide and the disease or disorder against which the mammalian subject is being vaccinated or treated is a mycobacterial or a bacterial infection. In this example, the peptide may be any appropriate peptide (antigen/epitope) that is present in a mycobacteria or bacteria against which the mammalian subject is being vaccinated or treated. In this context, the peptide may be a peptide from a mycobacteria or bacteria selected from the group consisting of: Mycobacterium tuberculosis (the causative agent of tuberculosis), Borrelia burgdorferi (the causative agent of lyme disease), Bacillus anthracis (the causative agent of anthrax), and Clostridium tetani (the causative agent of tetanus). Suitable peptides are well known to a person of skill in the art.

In another example, the non-mammalian peptide sequence is a yeast peptide and the disease or disorder against which the mammalian subject is being vaccinated or treated is a yeast infection. In this example, the peptide may be any appropriate peptide (antigen/epitope) that is present in a yeast against which the mammalian subject is being vaccinated or treated. In this context, the peptide may be a peptide from Candida albicans. Suitable peptides are well known to a person of skill in the art.

In a further example, the non-mammalian peptide sequence is a parasite peptide and the disease or disorder against which the mammalian subject is being vaccinated or treated is a parasite infection. In this example, the peptide may be any appropriate peptide (antigen/epitope) that is present in a parasite against which the mammalian subject is being vaccinated or treated. In this context, the peptide may be a peptide from a parasite selected from the group consisting of: Plasmodium (the causative agent of malaria), Leishmania (the causative agent of leishmaniasis), Schistosoma (the causative agent of Schistosomiasis), hook worms and ticks. Suitable peptides are well known to a person of skill in the art.

In one example, the non-mammalian peptide sequence is a viral peptide and the disease or disorder against which the mammalian subject is being vaccinated or treated is a viral infection. In this example, the viral peptide may be any appropriate peptide (antigen/epitope) that is present in a virus against which the mammalian subject is being vaccinated or treated. In this context, the viral peptide may be a peptide from a virus selected from the group consisting of but not limited to corona virus, influenza virus, respiratory syncytial human deficiency virus, Dengue virus, human papilloma virus, herpes simplex virus, varicella-zoster virus, mumps virus, measles virus, Rota virus, Noro virus, zika virus, Ebola virus, hepatitis a virus, hepatitis b virus, hepatitis c virus, and Epstein-Barr virus. Suitable peptides are well known to a person of skill in the art and include, for example, corona virus spike protein (e.g. peptides from the S1 or S2 subunits of the spike protein, such as peptides from the receptor binding domain (RBD) or fusion domain), or influenza hemagglutinin or neuraminidase. In this example, where the viral infection is influenza virus, the influenza virus peptide may be a H1 influenza virus peptide, a N1 influenza virus peptide, or a H1/N1 influenza virus peptide. For example, the influenza virus peptide may comprise the sequence of SEQ ID NO:42 (H1), 43 (N1) or 44 (H1N1). The immune stimulating peptide may comprise any immune stimulating peptide disclosed herein, preferably CDP (SEQ ID NO:5). Accordingly, the fusion polypeptide may comprise the sequence of SEQ ID NO: 45 (CDP-H1), 46 (CDP-N1) or 47 (CDP-H1N1).

In one example, the non-mammalian peptide sequence is a SARS-CoV-2 peptide (antigen/epitope) and the disease or disorder is a SARS-CoV-2 infection. For example, the SARS-CoV-2 peptide (antigen/epitope) may be a SARS-CoV-2 spike protein peptide (antigen/epitope). For example, the spike protein peptide may be a S1 subunit peptide or a S2 subunit peptide. In another example, the peptide may be a fusion domain peptide. In a further example, the SARS-CoV-2 peptide (antigen/epitope) may be a SARS-CoV-2 receptor binding domain (RBD) peptide (antigen/epitope), e.g. the RBD of the spike protein described in Wrapp et al., 2020. RBD is responsible for cell entry and subsequent infection through binding to angiotensin converting enzyme 2 (ACE-2) expressed by host cells. The vast majority of neutralizing antibodies detected in the serum of Covid-19 patients are directed against RBD. In this example, the SARS-CoV-2 peptide may comprise any one of more of: an RBM domain (amino acids 438-506 of the S1 subunit), an SD1 domain (amino acids 527-562 of the S1 subunit), an SD2 domain (amino acids 618-646 of the S1 subunit), an S1/S2 domain (amino acids 674-696 of the S2 subunit), FP+S2′ domain (amino acids 786-848 of the S2 subunit) and a HR1 domain (amino acids 912-943 of the S2 subunit). A RBM domain may comprise the sequence of SEQ ID NO: 69. An SD1 domain may comprise the sequence of SEQ ID NO: 70. An SD2 domain may comprise the sequence of SEQ ID NO: 71. An S1/S2 domain may comprise the sequence of SEQ ID NO:72. An FP domain may comprise the sequence of SEQ ID NO: 73. An HR1 domain may comprise the sequence of SEQ ID NO: 74. In one example, the SARS-CoV2 peptide may comprise the sequence of SEQ ID NO: 39, 40 or 41. The immune stimulating peptide may be any immune stimulating peptide disclosed herein, preferably CDP (SEQ ID NO:5). Accordingly, the fusion polypeptide may comprise the sequence of SEQ ID NO: 63 (CDP-Complete), SEQ ID NO: 64 (CDP-Binding) or SEQ ID NO: 65 (CDP-Fusion). The DNA sequence encoding the fusion polypeptide may for example, comprise the sequence of SEQ ID NO: 66 (CDP-Complete), 67 (CDP-Binding) or 68 (CDP-Fusion).

As would be clear to a person of skill in the art, the non-mammalian peptide sequence may include one or more antigens/epitopes. For example, the non-mammalian peptide sequence may comprise two or more SARS-CoV-2 antigens/epitopes. Accordingly, where reference is made to e.g. the non-mammalian peptide sequence being a SARS-CoV-2 peptide (antigen/epitope), this encompasses non-mammalian peptide sequences that comprise one or more, e.g. two or more, SARS-CoV-2 antigens/epitopes. The SARS-CoV-2 antigens/epitopes within the non-mammalian peptide sequence may be directly adjacent to each other, overlapping, or may be spatially separate within the non-mammalian peptide sequence.

In one example, the RBD peptide (antigen/epitope) comprises an amino acid sequence shown in SEQ ID NO:1, or a functional amino acid variant (or functional fragment) thereof. Such variants may be naturally occurring (e.g., allelic), synthetic, or synthetically improved functional variants of SEQ ID NO:1. The term “variant” also encompasses homologues.

Functional variants may typically contain only conservative amino acid substitutions of one or more amino acids of SEQ ID NO:1, or substitution, deletion or insertion of non-critical amino acids in non-critical regions of the protein. A functional variant of SEQ ID NO:1 may therefore be a conservative amino acid sequence variant of SEQ ID NO:1, wherein the variant still comprises the sequence elements necessary to obtain protective antibodies.

Methods for identifying functional and non-functional variants (e.g. functional and non-functional allelic variants) are well known to a person of ordinary skill in the art.

The RBD peptide described herein may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO: 1 or portions or fragments thereof. Suitably, percent identity can be calculated as the percentage of identity to the entire length of the reference sequence (e.g., SEQ ID NO:1), or portions or fragments thereof.

In another example, the non-mammalian peptide sequence is an influenza virus peptide and the disease or disorder is influenza. The term “influenza” as used herein comprises all types of influenza infections that can be caused by any of the four types of influenza virus, a, b, c, and d.

The non-mammalian peptide sequence itself may be highly immunogenic or moderately immunogenic. Alternatively, it may have low immunogenicity, or it may not be immunogenic at all. Due to the second part of the fusion polypeptide, the immune stimulating peptide, the immune response against the non-mammalian peptide sequence will be boosted relative to the immune response that would be elicited by the non-mammalian peptide sequence alone.

The “immune simulating peptide sequence” that is responsible for triggering, eliciting, enhancing, boosting the immune response against the non-mammalian peptide sequence may also be referred to as an “immune activating peptide”.

The immune stimulating peptide sequence may be synthetic or artificial sequence. Alternatively, the immune stimulating peptide sequence may be a sequence that occurs in nature. In one example, as demonstrated in the examples below, the immune stimulating peptide sequence may be a sequence that originates from one or more bacterial sequences. An immune stimulating peptide sequence that comprises one or more bacterial sequences is advantageous, as it will be recognized as “foreign” by the mammalian immune system. This renders the immune stimulating peptide sequence immunogenic.

The immunogenicity of the immune stimulating peptide sequence may be optimized by the ratio of bulky hydrophilic or charged amino acids to the total amino acids that is present within the immune stimulating peptide sequence. The inventors have found that the ratio of bulky hydrophilic or charged amino acids to the total amino acids within the immune stimulating peptide sequence provides a reliable measure of the efficacy of the immune stimulating peptide sequence in stimulating an immune response to a linked antigen of interest. An immune stimulating peptide sequence having a ratio of bulky hydrophilic or charged amino acids to the total amino acids of less than or equal to 0.55 increases the solubility of the whole fusion polypeptide, which in turn results in better recognition by the immune system and increased immunogenicity in vivo. Accordingly, in the context of the invention, it is important that the ratio of bulky hydrophilic or charged amino acids to the total amino acids of the immune stimulating peptide should be less than or equal to 0.55.

The term “bulky hydrophilic or charged amino acids” as used herein refers to a group of amino acid residues, consisting of histidine, glutamic acid, arginine, glutamine, aspartic acid and lysine.

The ratio of bulky hydrophilic or charged amino acids to the total amino acids is calculated by dividing the total number of bulky hydrophilic or charged amino acids by the number of total amino acids in the immune stimulating peptide sequence. For example, if the immune stimulating peptide sequence has a total of 100 amino acids, of which 20 fall under the definition of “bulky hydrophilic or charged amino acids” as described above, the ratio would be 20:100=0.20. This ratio is calculated on the basis of the amino acids in the core immune stimulating peptide sequence only. For the avoidance of doubt, auxiliary tags that are present within the fusion polypeptide (e.g. N-terminal or C terminal tags such as His tags) do not form part of the core immune stimulating peptide sequence (as they are typically removed from the final fusion protein before it is administered to a patient), thus should be disregarded when calculating this ratio (even if they are immediately adjacent to the core immune stimulating peptide sequence). Such auxiliary tags are well known to a person of skill in the art, and will be readily identifiable to them. In addition, any auxiliary linkers (e.g. GS linkers, such as GSGS or GSGSGS (SEQ ID NO:27) linkers) do not form part of the core immune stimulating peptide sequence, thus should be disregarded when calculating this ratio (even if they are immediately adjacent to the core immune stimulating peptide sequence e.g. linking the core immune stimulating peptide sequence to the antigen of interest). Such auxiliary linkers are well known to a person of skill in the art, and will be readily identifiable to them. Finally, the first methionine (encoded by the AUG start codon) is also discounted from the ratio calculation.

As shown herein, the sequence HHAH can serve as a purification tag (as an alternative to a His tag (HHHHHH; SEQ ID NO:26)). The sequence HHAH is a naturally occurring sequence originating from the P30131|HYPF_ECOLI Carbamoyltransferase HypF OS=Escherichia coli (strain K12) protein. As such, there is no need to remove this sequence before obtaining the final fusion polypeptide (as would have to be done with a His tag). For this reason, the HHAH sequence is considered as part of the sequence when calculating the ratio of bulky hydrophilic or charged amino acids to the total amino acids in the immune stimulating peptide sequence, whereas a His tag is not.

The total length of the immune stimulating peptide sequence may also be considered when choosing a suitable immune stimulating peptide sequence for inclusion into the fusion polypeptides described herein. For example, a short immune stimulating peptide may be used to reduce/minimize the immune response to the immune stimulating peptide itself (shifting the focus of the immune response to the antigen of interest instead).

In one example, the immune stimulating peptide sequence is at least 40 amino acids long and is no more than 150 amino acids long. For example, the immune stimulating peptide sequence may be at least 40 amino acids, at least 45 amino acids, at least 50 amino acids, or at least 55 amino acids long (but no more than 150 amino acids long). In a further example, the immune stimulating peptide sequence may be at least 60 amino acids, at least 65 amino acids or at least 70 amino acids long (but no more than 150 amino acids long).

In one example, the immune stimulating peptide sequence is no more than 150 amino acids, no more than 145 amino acids, or no more than 140 amino acids long (with a minimum length of 40 amino acids).

In a further example, the immune stimulating peptide sequence is no more than 135 amino acids, no more than 130 amino acids, no more than 125 amino acids long, or no more than 120 amino acids long (with a minimum length of 40 amino acids).

In a further example, the immune stimulating peptide sequence is no more than 100 amino acids (with a minimum length of 40 amino acids).

The length of the immune stimulating peptide sequence may also depend on the length of the peptide sequence of interest. When the sequence of the non-mammalian or mammalian peptide sequence is short, the attached immune stimulating peptide should be less than 50% of the total protein. As a guideline, a suitable length of the immune stimulating peptide sequence may be a length that is about ⅓ (one third) of the non-mammalian or mammalian peptide sequence (i.e. the antigen of interest). However, when selecting the length, the overall solubility of the fusion protein should also be considered.

The isoelectric point of the immune stimulating peptide sequence may also be optimized for inclusion into a fusion polypeptide as described herein. As described elsewhere herein, the pI of a protein affects its solubility at physiological pH. In one example, the immune stimulating peptide sequence has a predicted isoelectric point (pI) of less than or equal to 4.

For example, the immune stimulating peptide sequence may have a predicted pI of less than or equal to 3.96, of less than or equal to 3.8, such as of less than or equal to 3.78, 3.7, or 3.63.

In one example, the immune stimulating peptide sequence comprises the amino acid sequence of SEQ ID NO:4 or a conservative amino acid variant thereof. This sequence increases the solubility of the fusion polypeptide because amino acids D (aspartic acid) and E (glutamic acid) have a very low pI and are negatively charged at physiological pH (pH 7.4). The immune stimulating peptide sequence may comprise an amino acid sequence having at least about 80%, 90%, 91%, 92%, 93%, 94%, 95%, 96%, 97%, 98%, 99%, 100% identity to the amino acid of SEQ ID NO:4, or portions or fragments thereof. Suitably, percent identity can be calculated as a percentage of identity to the entire length of the reference sequence (e.g., SEQ ID NO:4), or portions or fragments thereof.

When replacing amino acids this may result in a change of the pI and therefore a person skilled in the art would be aware of only making amendments to this sequence that would still provide the desired overall pI of the fusion polypeptide.

As described elsewhere herein, the pI of the fusion polypeptide described herein can be an important feature, as a low pI can increase solubility and immunogenicity. The immune simulating peptide sequence may be selected to provide an overall pI for the fusion polypeptide that results in high solubility and immunogenicity under physiological conditions. Accordingly, for antigens of interest (e.g. non-mammalian peptide sequences or mammalian peptide sequences) that have a relatively high predicted pI (e.g. a predicted pI of around pH 7.4, i.e., physiological conditions, or above), which would be expected have low solubility/immunogenicity in vivo, an immune simulating peptide sequence may be selected with a low pI to counteract this and lower the overall pI of the fusion polypeptide to more desirable levels (resulting in higher solubility and immunogenicity for the antigen of interest (e.g. non-mammalian peptide sequence or mammalian peptide sequence)). Suitable combinations of immune simulating peptide sequences and antigens of interest (e.g. non-mammalian peptide sequences or mammalian peptide sequences) are provided herein.

Several immune stimulating peptide sequences having a ratio of bulky hydrophilic or charged amino acids to the total amino acids of less than or equal to 0.55 are described in detail herein (e.g. wherein the immune stimulating peptide sequence may be at least 40 amino acids long and no more than 150 amino acids long and/or have a predicted pI of less than or equal to 4). Alternative appropriate immune stimulating peptide sequences may be readily identified by a person of skill in the art, using the features described herein.

For example, immunogenic bacterial sequences (or a composite of such bacterial sequences) may provide a suitable immune stimulating peptide sequence having a ratio of bulky hydrophilic or charged amino acids to the total amino acids of less than or equal to 0.55 (e.g. wherein the immune stimulating peptide sequence may be at least 40 amino acids long and no more than 150 amino acids long and/or have a predicted pI of less than or equal to 4).

Examples of such immune stimulating peptide sequences are provided elsewhere herein, and include the bacterial protein thioredoxin (TRX UNIPROT #P0AA25), a truncated version thereof (TRXtr), type-1 fimbrial protein, chain A (TFP, UNIPROT #P04128), a variant thereof (TFPv), and immune stimulating peptides 1 to 4. These are described in detail further below.

Accordingly, in one example the fusion polypeptide for use in vaccinating or treating a mammalian subject comprises:
(6) a non-mammalian peptide sequence, wherein the peptide sequence is a sequence that is associated with a disease or disorder against which the mammalian subject is being vaccinated or treated; and
(ii) an immune stimulating peptide sequence selected from the group consisting of:
   (a) a sequence comprising SEQ ID NO:10 or conservative amino acid variants thereof, optionally wherein the immune stimulating peptide sequence is no longer than 80 amino acids;
   (b) a sequence comprising SEQ ID NO:12 or conservative amino acid variants thereof;
   (c) a sequence comprising SEQ ID NO:5 or conservative amino acid variants thereof;
   (d) a sequence comprising SEQ ID NO:6 or conservative amino acid variants thereof;
   (e) a sequence comprising SEQ ID NO:7 or conservative amino acid variants thereof; or
   (f) a sequence comprising SEQ ID NO:8 or conservative amino acid variants thereof.

In one example, the fusion polypeptide comprises:
(i) a non-mammalian peptide sequence comprising SEQ ID NO:1, and
(ii) an immune stimulating peptide sequence having a ratio of bulky hydrophilic or charged amino acids to remaining amino acids of less than or equal to 0.55.

In one example, the fusion polypeptide comprises:
(i) a non-mammalian peptide sequence comprising SEQ ID NO:1, and
(ii) an immune stimulating peptide sequence selected from the group consisting of:
   (a) a sequence comprising SEQ ID NO:10 or conservative amino acid variants thereof, optionally wherein the immune stimulating peptide sequence is no longer than 80 amino acids;
   (b) a sequence comprising SEQ ID NO:12 or conservative amino acid variants thereof;
   (c) a sequence comprising SEQ ID NO:5 or conservative amino acid variants thereof;
   (d) a sequence comprising SEQ ID NO:6 or conservative amino acid variants thereof;
   (e) a sequence comprising SEQ ID NO:7 or conservative amino acid variants thereof; or
   (f) a sequence comprising SEQ ID NO:8 or conservative amino acid variants thereof.

The immune stimulating peptide sequence may, in some advantageous examples, comprise at least one (predicted) T-cell epitope.

The term “T-cell epitope” as used herein refers to T-cell epitopes, in particular to CD4+ T-cell epitopes. In other words, they are epitopes that are recognized and bound by T cell receptors (TCRs) found on the surface of CD4+ T cells. T-cell epitopes are presented by major histocompatibility complex (MHC) molecules to TCRs. In humans, professional antigen presenting cells are specialized to present T-cell epitopes in the context of MHC II, whereas most nucleated somatic cells present T-cell epitopes in the context of MHC II. MHC class II molecules present peptides, 13-17 amino acids in length, and non-classical MHC molecules also present non-peptic epitopes such as glycolipids.

For predicting whether a peptide sequence is or comprises a T-cell epitope online tools can be used like for example provided on the website:

http://www.cbs.dtu.dk/services/NetMHCpan/.

The immune stimulating peptide sequence may comprise one, two, three, four or more (predicted) T-cell epitopes. For example, the immune stimulating peptide sequence comprises one or two (predicted) T-cell epitopes.

In one example, the immune stimulating peptide sequence comprises one or two (predicted) CD4+ T-cell epitopes.

Within the immune stimulating peptide sequence the (predicted) T-cell epitopes may either be directly adjacent/fused to each other or they may be separated by a linker or any other filling sequence.

In one example, the immune stimulating peptide sequence comprises a (predicted) T-cell epitope selected from a MsyB protein (P257381MSYB_ECOLI Acidic protein MsyB OS=Escherichia coli (strain K12)) and/or a RRAB protein (P0AF90IRRAB_ECOLI Regulator of ribonuclease activity B OS=Escherichia coli (strain K12)). MsyB and RRAB proteins originate from E. coli, and both are considered as acidic proteins.

For example, the following (predicted) CD4+ T-cell epitopes within the RRAB protein (based on http://www.cbs.dtu.dk/services/NetMHCpan/) may be present within the immune stimulating peptide sequence: KLGYEVYEVTDPEELEVE (SEQ ID NO:13), FKLGYEVYEVTDPEELEV (SEQ ID NO:14) and/orYEVTDPEEL (SEQ ID NO:15).

Alternatively, or in addition, one or more of the following (predicted) CD4+ T-cell epitopes within the MsyB protein (based on http://www.cbs.dtu.dk/services/NetMHCpan/) may be present within the immune stimulating peptide sequence: EGEFQLEPPLDTEEG (SEQ ID NO:16), AQKYVLQDGDIMWQV (SEQ ID NO:17), DEGEFQLEPPLDTEE (SEQ ID NO:18), NAQKYVLQDGDIMWQ (SEQ ID NO:19), EPPLDTEEGRAADE (SEQ ID NO:20), NPGIDAEDANVQQFN (SEQ ID NO:21), LEPPLDTEEGRAAD (SEQ ID NO:22), FQLEPPLDT YVLQDGDIM (SEQ ID NO:23), LDTEEGRAA (SEQ ID NO:24) and/or IDAEDANVQ (SEQ ID NO:25).

As would be clear to a person of skill in the art, the immune stimulating peptide sequence may comprise two or more of these T-cell epitopes. For example, two or more of the T-cell epitopes may be located directly adjacent to each other, or may be partially overlapping (where their sequence allows) within the immune stimulating peptide sequence; e.g. as shown in NPGIDAEDANVQQFNAQKYVLQDGDIMWQV (SEQ ID NO:2) and/or EGEFQLEPPLDTEEGRAAADE (SEQ ID NO:3).

The Immune stimulating peptide sequence as described herein may comprise one (predicted) T-cell epitope from the MsyB protein or one (predicted) T-cell epitope from the RRAB protein or it may comprise several (predicted) T-cell epitopes of the MsyB protein or several (predicted) T-cell epitopes of the RRAB protein, or it may comprise a combination of one or more (predicted) T-cell epitopes of either of the MsyB or RRAB protein.

For example, the immune stimulating peptide sequence may comprise one MsyB protein (predicted) T-cell epitope. In another example the immune stimulating peptide sequence may comprise two MsyB protein (predicted) T-cell epitopes.

In one example, the immune stimulating peptide sequence may comprise the amino acid sequence SEQ ID NO:2 and/or SEQ ID NO:3 or a conservative amino acid variant thereof.

An immune simulating peptide sequence may comprise an amino acid sequence having at least about 60%, 65%, 70%, 75%, 80%, 85%, 85%, 90%, 91%, 92%, 93%, 94%, 95%, 96%, 97%, 98%, 99% or 100% identity to the amino acid sequence of SEQ ID NO:2, or portions or fragments thereof, suitably, percent identity can be calculated as the percentage of identity to the entire length of the reference sequence (e.g., SEQ ID NO:2), or portions or fragments thereof.

An Immune simulating peptide sequence may comprise an amino acid sequence having at least about 60%, 65%, 70%, 75%, 80%, 85%, 85%, 90%, 91%, 92%, 93%, 94%, 95%, 96%, 97%, 98%, 99% or 100% identity to the amino acid sequence of SEQ ID NO:3, or portions or fragments thereof, suitably, percent identity can be calculated as the percentage of identity to the entire length of the reference sequence (e.g., SEQ ID NO:3), or portions or fragments thereof.

When replacing amino acids this may result in a change of the pI and therefore a person skilled in the art would be aware of only making amendments to this sequence that would still provide the desired overall pI of the fusion polypeptide.

In one example, the immune stimulating peptide sequence comprises both amino acid sequences SEQ ID NO:2 and SEQ ID NO:3. Optionally, when the immune stimulating peptide sequence comprises both amino acid sequences SEQ ID NO:2 and SEQ ID NO:3 these sequences are directly adjacent/fused to each other within the peptide sequence.

In one example, the immune stimulating peptide sequence comprises or consists of the amino acid sequence: DNNSLSQEVQNGSNHLENNQSQSNGGGSDSALSLSSKTAALAAATTVNDGSDGATSSAVG (CDP; SEQ ID NO:5), or a conservative amino acid variant thereof.

CDP comprises the following peptide stretches: NNSLSQEVQN (SEQ ID NO: 28) (10 aa) derived from ZapB (amino acids 36-45), NHLENNQSQSNGG (SEQ ID NO: 29) (13 aa) derived from IbpA (amino acids 21-33), SALSLSSTAALAAATTVN (SEQ ID NO:30) (18 aa) and GATSSAVG (SEQ ID NO:31) (8 aa) derived from TFP (amino acids 12-39 and 69-76 respectively). This is depicted below:

CDP (SEQ ID NO: 5):
ZapB IbpA TFP TFP
DNNSLSQEVQNGSNHLENNQSQSNGGGSDSALSLSSKTAALAAATTVNDGSDGATSSAVG

A total of five amino acids within CDP have bulky hydrophilic/charged side chains (2×E, 3×Q and 1×H). To increase the immunogenicity of CDP, some alanine or glycine residues were mutated to lysine or aspartic acid residues (underlined) to obtain a ratio of bulky hydrophilic/charged amino acid and remaining amino acids of 0.18 (11 bulky hydrophilic, charged aa/60 total aa). The peptide stretches were linked by GS-linkers (1×GS).

An immune simulating peptide sequence may therefore comprise an amino acid sequence having at least about 60%, 65%, 70%, 75%, 80%, 85%, 85%, 90%, 91%, 92%, 93%, 94%, 95%, 96%, 97%, 98%, 99% or 100% identity to the amino acid sequence of SEQ ID NO:5, or portions or fragments thereof, suitably, percent identity can be calculated as the percentage of identity to the entire length of the reference sequence (e.g., SEQ ID NO:5), or portions or fragments thereof. In another example, the immune stimulating peptide sequence comprises or consists of the amino acid sequence: HHAHTMYATLEEAIDAAREEFLADNPGIDAEDANVQQFNAQKYVLQDGDIMWQVEFFADEGEE GEDDEDFVDEDDD (IDP1; SEQ ID NO:6), or a conservative amino acid variant thereof.

IDP1 comprises the natural histidine-rich HHAH sequence of the E. coli protein P30131|HYPF_ECOLI Carbamoyltransferase HypF OS=Escherichia coli (strain K12) for purification purposes, the N-terminal part of the MsyB protein (amino acids 2 to 62) including the (predicted) T-cell epitope construct according to SEQ ID NO:2. In addition thereto it contains the amino acid sequence according to SEQ ID NO:4 of the RRAB protein. IDP1 has a predicted pI of 3.63 and a molecular weight of 8813.23 Da with a solubility of 0.815. The ratio of bulky hydrophilic or charged amino acids to the total amino acids is 0.47 (36 bulky hydrophilic, charged aa/76 aa in total).

An Immune simulating peptide sequence may comprise an amino acid sequence having at least about 60%, 65%, 70%, 75%, 80%, 85%, 85%, 90%, 91%, 92%, 93%, 94%, 95%, 96%, 97%, 98%, 99% or 100% identity to the amino acid sequence of SEQ ID NO:6, or portions or fragments thereof, suitably, percent identity can be calculated as the percentage of identity to the entire length of the reference sequence (e.g., SEQ ID NO:6), or portions or fragments thereof.

In a further example, the immune stimulating peptide sequence comprises or consists of the amino acid sequence: HHAHNPGIDAEDANVQQFNAQKYVLQDGDIMWQVEGEFQLEPPLDTEEGRAAADEDDEDFVD EDDD (IDP2; SEQ ID NO:7), or a conservative amino acid variant thereof.

IDP2 comprises the natural histidine-rich HHAH sequence of the E. coli protein P30131|HYPF_ECOLI Carbamoyltransferase HypF OS=Escherichia coli (strain K12) for purification purposes, the two (predicted) T-cell epitope constructs of the MsyB protein according to SEQ ID NO:2 and SEQ ID NO:3, fused to each other. Additionally, the sequence comprises the amino acid sequence according to SEQ ID NO:4 fused to the (predicted) T-cell epitope construct according to SEQ ID NO:3.

IDP2 has a predicted pI of 3.70 in the molecular weight of 7616.93 Da with a solubility of 0.815. The ratio of bulky hydrophilic or charged amino acids to the total amino acids is 0.45 (30 bulky hydrophilic, charged aa/66 aa in total).

An immune simulating peptide sequence may comprise an amino acid sequence having at least about 60%, 65%, 70%, 75%, 80%, 85%, 85%, 90%, 91%, 92%, 93%, 94%, 95%, 96%, 97%, 98%, 99% or 100% identity to the amino acid sequence of SEQ ID NO:7, or portions or fragments thereof, suitably, percent identity can be calculated as the percentage of identity to the entire length of the reference sequence (e.g., SEQ ID NO:7), or portions or fragments thereof.

In a further example, the immune stimulating peptide sequence comprises or consists of the amino acid sequence: HHAHNPGIDAEDANVQQFNAQKYVLQDGDIMWQVDDEDFVDEDDD (IDP3; SEQ ID NO:8), or a conservative amino acid variant thereof.

IDP3 comprises the natural histidine-rich HHAH sequence of the E. coli protein P30131|HYPF_ECOLI Carbamoyltransferase HypF OS=Escherichia coli (strain K12) for purification purposes, the first (predicted) T-cell epitope construct of the MysB protein according to SEQ ID NO:2 fused to the amino acid sequence according to SEQ ID NO:4.

IDP3 has a predicted pI of 3.78, a molecular weight of 5331.57 Da with a solubility of 0.855. The ratio of bulky hydrophilic or charged amino acids to the total amino acids is 0.44 (20 bulky hydrophilic, charged aa/45 aa in total).

An immune simulating peptide sequence may comprise an amino acid sequence having at least about 60%, 65%, 70%, 75%, 80%, 85%, 85%, 90%, 91%, 92%, 93%, 94%, 95%, 96%, 97%, 98%, 99% or 100% identity to the amino acid sequence of SEQ ID NO:8, or portions or fragments thereof, suitably, percent identity can be calculated as the percentage of identity to the entire length of the reference sequence (e.g., SEQ ID NO:8), or portions or fragments thereof.

All four sequences according SEQ ID NO:5, SEQ ID NO:6, SEQ ID NO:7, and SEQ ID NO:8 (and conservative amino acid variants thereof) represent a core immune stimulating peptide sequence as described herein. Ancillary tags and linkers may be added to these sequences as described elsewhere herein. For example, a natural histidine rich sequence like HHAH (from the E. coli protein P30131, HYPF_E. coli) if not already present or HHHHHH may be added at the N-terminus of the sequences. Furthermore, a person skilled in the art would be aware that a methionine at the beginning of each sequence will be required in order to obtain a synthesized protein. In these examples, as described elsewhere herein, when calculating the ratio of bulky hydrophilic or charged amino acids to the total amino acids within the immune stimulating peptide sequence, neither the methionine nor the His tag would be taken into account. The histidine rich sequence HHAH would, however, be taken into account.

Alternative immune stimulating peptide sequences that can be included in a fusion polypeptide as described herein include: thioredoxin (TRX), a truncated version of thioredoxin (TRXtr), Type-1 fimbrial protein (A chain) (TFP), or variant Type-1 fimbrial protein (A chain) (TFPv). The usefulness of these immune stimulating peptide sequences as fusion polypeptides with self-antigens has been described previously. A few details on these sequences are provided below

Thioredoxin (TRX; 11.8 kDa)
(SEQ ID NO: 9)
SDKIIHLTDDSFDTDVLKADGAILVDFWAEWCGPCKMIAPILDEIADEYQ
GKLTVAKLNIDQNPGTAPKYGIRGIPTLLLFKNGEVAATKVGALSKGQLK
EFLDANLA (108 aa)

For this immune stimulating peptide sequence, the amino acids with bulky hydrophilic/charged side chains within the TRX protein sequence are: 1×H, 5×E, 1×R, 3×Q, 11×D, 10×K. In total there are 31 amino acids that have bulky hydrophilic/charged side chains; therefore, the ratio of bulky hydrophilic/charged amino acid to total amino acids within the TRX protein is 31aa/108aa=0.29.

Thioredoxin trunc (TRXtr; 6.2 kDa)
(SEQ ID NO: 10)
GKLTVAKLNIDQNPGTAPKYGIRGIPTLLLFKNGEVAATKVGALSKGQLK
EFLDANLA (58 aa)

In TRXtr the C-terminal part of the full-length thioredoxin sequence (aa 51-109) is only used, (i.e. the last 58 C-terminal amino acids of the protein). An additional methionine is added at the N-terminus and a GS-linker (4× glycine-serine) and His-tag (6× histidine) at the C-terminal end of the TRXtr protein sequence. A GS-linker is included to provide the fusion protein constructs with flexibility and the His-tag for purification purposes. Compared to full-length thioredoxin, the TRXtr sequence only contains 14 amino acids with bulky hydrophilic/charged side chains (2×E, 2×Q, 1×R, 2×D and 7×K), giving a ratio of bulky hydrophilic/charged amino acid to total amino acids of 14aa/58aa=0.24.

Type-1 fimbrial protein, A chain
(TFP; 13 kDa)
(SEQ ID NO: 11)
AATTVNGGTVHFKGEVVNAACAVDAGSVDQTVQLGQVRTASLAQEGATSS
AVGFNIQLNDCDTNVASKAAVAFLGTAIDAGHTNVLALQSSAAGSATNVG
VQILDRTGAALTLDGATFSSETTLNNGTNTI (131 aa)

Of the full-length TFP protein only the protein sequence without the signaling peptide is used (aa 24-182; 13 kDa) and a N-terminal methionine is added. The number of amino acids with bulky hydrophilic/charged side chains within the TFP protein sequence is: 2×H, 3×E, 2×R, 7×Q, 7×D, 2×K. In total there are 23 amino acids which have bulky hydrophilic/charged side chains; therefore the ratio of bulky hydrophilic/charged amino acid to total amino acids within the TFP protein is 23aa/131aa=0.18.

variant Type-1 fimbrial protein, A chain
(TFPv; 13.3 kDa)
(SEQ ID NO: 12)
KATTVNGGTVHFKGEVVNAACAVDAGSVDQTVQLGQVRTASLAQEGATSS
KVGFNIQLNDCDTNVASKAAVAFLGTKIDAGHTNVLALQSSAAGSDTNVG
VQILDRTGAALTLDGATFSSETTLNNDTNTI (131 aa)

A TFPvariant (TFPv) protein was obtained by mutating a number of alanine (Ala, A) or glycine residues (Gly, G) to lysine or aspartic acid (underlined); thereby resulting in a TFP protein with higher immunogenicity. Amino acids with bulky hydrophilic/charged side chains within the TFPvariant protein sequence are: 2×H, 3×E, 2×R, 7×Q, 8×D, 5×K. In total there are 27 amino acids which have bulky hydrophilic/charged side chains; therefore the ratio of bulky hydrophilic/charged amino acid to total amino acids within the TFP protein is 27aa/131aa=0.21

### Fusion Polypeptides

According to a further aspect of the present invention a fusion polypeptide is provided comprising:
(6) a non-mammalian peptide sequence, wherein the peptide sequence is a sequence that is associated with a disease or disorder against which the mammalian subject is being vaccinated or treated; and
(ii) an immune stimulating peptide sequence having a ratio of bulky hydrophilic or charged amino acids to total amino acids of less than or equal to 0.55;
   wherein the non-mammalian peptide sequence is linked to the immune stimulating peptide sequence and wherein the immune stimulating peptide sequence comprises the amino acid sequence:
a) HHAHTMYATLEEAIDAAREEFLADNPGIDAEDANVQQFNAQKYVLQDGDIMWQVE FFADEGEEGEDDEDFVDEDDD (IDP1; SEQ ID NO:6), or a conservative amino acid variant thereof; or
b) HHAHNPGIDAEDANVQQFNAQKYVLQDGDIMWQVEGEFQLEPPLDTEEGRAAAD EDDEDFVDEDDD (IDP2; SEQ ID NO:7), or a conservative amino acid variant thereof; or
c) HHAHNPGIDAEDANVQQFNAQKYVLQDGDIMWQVDDEDFVDEDDD (IDP3; SEQ ID NO:8), or a conservative amino acid variant thereof; or
d) DNNSLSQEVQNGSNHLENNQSQSNGGGSDSALSLSSKTAALAAATTVNDGSDGA TSSAVG (CDP; SEQ ID NO:5), or a conservative amino acid variant thereof.

In one example,
a) the non-mammalian peptide sequence is a viral peptide and the disease or disorder is a viral infection; or
b) the non-mammalian peptide sequence is a mycobacterial or a bacterial peptide and the disease or disorder is a mycobacterial or a bacterial infection; or
c) the non-mammalian peptide sequence is a yeast peptide and the disease or disorder is a yeast infection; or
d) the non-mammalian peptide sequence is a parasite peptide and the disease or disorder is a parasite infection.

Further details with regard to the viral, bacterial, mycobacterial, yeast and parasite peptides and corresponding infections are described elsewhere herein and equally apply here.

In one example, the non-mammalian peptide sequence is a SARS-CoV-2 peptide and the disease or disorder is a SARS-CoV-2 infection.

In one example, the non-mammalian peptide sequence is a SARS-CoV-2 peptide (antigen/epitope) and the disease or disorder is a SARS-CoV-2 infection. For example, the SARS-CoV-2 peptide (antigen/epitope) may be a SARS-CoV-2 spike protein peptide (antigen/epitope). For example, the SARS-CoV-2 peptide (antigen/epitope) may be a SARS-CoV-2 receptor binding domain (RBD) peptide (antigen/epitope), e.g. the RBD of the spike protein described in Wrapp et al., 2020.

In one example, the RBD peptide (antigen/epitope) comprises an amino acid sequence shown in SEQ ID NO:1, or a functional amino acid variant (or functional fragment) thereof. Such variants may be naturally occurring (e.g., allelic), synthetic, or synthetically improved functional variants of SEQ ID NO:1. The term “variant” also encompasses homologues.

Suitable RBD peptides and functional variants are described elsewhere herein and equally apply here.

In another example, the non-mammalian peptide sequence is an influenza virus peptide and the disease or disorder is influenza. The term “influenza” is defined elsewhere herein, and applies equally to this aspect.

Preferably, the fusion polypeptide elicits in a subject a clinically relevant immune response in the sense that a serum level of immunoglobulins against said non-mammalian peptide sequence of at least ELISA-detectable levels of immunoglobulins directed against the non-mammalian peptide sequence is achieved. ELISA-detectable levels refer to statistically significant detection above background or above a control or threshold value in an ELISA protocol. Preferably, said relevant immune response involves a serum level of immunoglobulins against said non-mammalian peptide sequence of at least 5 times the signal measured in control serum of non-vaccinated individuals, detected by ELISA, preferably identical with at least 0.1% of the total serum immunoglobulin G (IgG) level.

According to another aspect of the present invention a fusion polypeptide is provided comprising
(6) a mammalian peptide sequence, wherein the peptide sequence is a sequence that is associated with a disease or disorder against which the mammalian subject is being vaccinated or treated; and
(ii) an immune stimulating peptide sequence having a ratio of bulky hydrophilic or charged amino acids to total amino acids of less than or equal to 0.55;
   wherein the mammalian peptide sequence is linked to the immune stimulating peptide sequence and wherein the immune stimulating peptide sequence comprises the amino acid sequence:
a) HHAHTMYATLEEAIDAAREEFLADNPGIDAEDANVQQFNAQKYVLQDGDIMWQVE FFADEGEEGEDDEDFVDEDDD (IDP1; SEQ ID NO:6), or a conservative amino acid variant thereof; or
b) HHAHNPGIDAEDANVQQFNAQKYVLQDGDIMWQVEGEFQLEPPLDTEEGRAAAD EDDEDFVDEDDD (IDP2; SEQ ID NO:7), or a conservative amino acid variant thereof; or
c) HHAHNPGIDAEDANVQQFNAQKYVLQDGDIMWQVDDEDFVDEDDD (IDP3; SEQ ID NO:8), or a conservative amino acid variant thereof.

In one embodiment the mammalian peptide sequence is associated with tumor angiogenesis. Several examples of suitable peptides associated with tumor angiogenesis are described in detail elsewhere herein.

In one embodiment the mammalian peptide sequence is vimentin (Vim).

In one embodiment, the mammalian peptide sequence is apelin, such as mouse apelin.

In one embodiment, the mammalian peptide sequence is notum, such as mouse notum.

In one embodiment, the mammalian peptide sequence is timp1, such as mouse timp1.

With a fusion polypeptide comprising a mammalian peptide sequence against which an immune response should be elicited, it is possible to trigger the immune system to produce anti-self antibodies when administering the fusion polypeptide to a subject.

Preferably, the fusion polypeptide elicits in a subject a clinically relevant immune response in the sense that a serum level of immunoglobulins against said mammalian peptide sequence of at least ELISA-detectable levels of immunoglobulins directed against the mammalian peptide sequence is achieved. ELISA-detectable levels refer to statistically significant detection above background or above a control or threshold value in an ELISA protocol. Preferably, said relevant immune response involves a serum level of immunoglobulins against said mammalian peptide sequence of at least 5 times the signal measured in control serum of non-vaccinated individuals, detected by ELISA, preferably identical with at least 0.1% of the total serum immunoglobulin G (IgG) level.

The fusion polypeptide described herein may have a predicted isoelectric point (pI) of less than or equal to 6. As mentioned elsewhere herein, the isoelectric point (pI) of a polypeptide is directly correlated to its solubility and has a direct impact on the ability of the fusion polypeptide to trigger or enhance an immune response against the antigen of interest. Isoelectric point is defined elsewhere herein, which applies equally to this aspect.

Fusion polypeptides as described herein with a pI that is less than physiological pH (of around 7.4) will have increased solubility in vivo, which is desirable when eliciting an immune response.

In one example, the predicted pI of the fusion polypeptide described herein is less than or equal to 5.5 or less than or equal to 5.

In another example, the predicted pI of the fusion polypeptide described herein is less than or equal to 4.5, or less than or equal to 4.

The immune stimulating peptide sequences of SEQ ID NO: 5 to 8 (and variants thereof) have been discussed in detail elsewhere herein. These details apply equally to this aspect.

Suitably the immune stimulating peptide sequence is at least 40 amino acids long and is no more than 150 amino acids long.

In one example, the immune stimulating peptide sequence is at least 40 amino acids long and is no more than 150 amino acids long. For example, the immune stimulating peptide sequence may be at least 40 amino acids, at least 45 amino acids, at least 50 amino acids, or at least 55 amino acids long (but no more than 150 amino acids long). In a further example, the immune stimulating peptide sequence may be at least 60 amino acids, at least 65 amino acids or at least 70 amino acids long (but no more than 150 amino acids long).

In one example, the immune stimulating peptide sequence is no more than 150 amino acids, no more than 145 amino acids, or no more than 140 amino acids long (with a minimum length of 40 amino acids).

In a further example, the immune stimulating peptide sequence is no more than 135 amino acids, no more than 130 amino acids, no more than 125 amino acids long, or no more than 120 amino acids long (with a minimum length of 40 amino acids).

In a further example, the immune stimulating peptide sequence is no more than 100 amino acids (with a minimum length of 40 amino acids).

The length of the immune stimulating peptide sequence may also depend on the length of the peptide sequence of interest. Details of this are provided elsewhere herein and apply equally to this aspect.

The isoelectric point of the immune stimulating peptide sequence may also be optimized for inclusion into a fusion polypeptide as described herein. As described elsewhere herein, the pI of a protein affects its solubility at physiological pH. In one example, the immune stimulating peptide sequence has a predicted isoelectric point (pI) of less than or equal to 4.

For example, the immune stimulating peptide sequence may have a predicted pI of less than or equal to 3.8, such as of less than or equal to 3.78, 3.7, or 3.63.

Any aspects discussed herein in respect of the immune stimulating peptide sequence and fusion polypeptide apply equally to these aspects.

### Nucleic Acids, Compositions and Kits

According to another aspect a nucleic acid is provided herein encoding a fusion polypeptide according to the invention, optionally wherein the nucleic acid is an expression vector.

A pharmaceutical composition is also provided herein, wherein the composition comprises a fusion polypeptide according to the invention, or a nucleic acid sequence or vector according to the invention and a pharmaceutically acceptable carrier, adjuvant, excipient or diluent. Compositions may routinely contain pharmaceutically acceptable concentrations of salt, buffering agents, preservatives, compatible carriers, supplementary immune potentiating agents, such as adjuvants and cytokines and optionally other therapeutic agents or compounds.

The pharmaceutical composition described herein may be used to vaccinate or treat a subject in need of an immune response against a certain peptide sequence. This can be immune responses triggered against non-mammalian or mammalian peptide sequences. Details with regard to the fusion polypeptide, the non-mammalian peptide sequence, the mammalian peptide sequence, the immune stimulating peptide sequence, diseases, disorders and ways of treatment are described herein and equally apply here. The definitions and explanations provided elsewhere herein equally apply to this aspect.

A kit Is also provided comprising the fusion polypeptide described herein, or a pharmaceutical composition comprising the fusion polypeptide or the nucleic acid as described herein.

### Medical Uses

Also provided herein is the use of a fusion polypeptide or a pharmaceutical composition described herein as a medicament. Also provided herein is the use of a fusion polypeptide or a pharmaceutical composition described herein for vaccination or treatment of a subject.

Details with regard to vaccination, treatment, subjects, diseases or disorders are described elsewhere herein and equally apply here.

A method of vaccinating or treating a subject is also provided herein, the method comprising administering a fusion polypeptide described herein or a pharmaceutical composition as described herein to the subject.

A method of vaccinating a subject against SARS-CoV-2 with a fusion polypeptide or pharmaceutical composition as described herein is provided herein as well. In particular a method of vaccinating or treating a subject is provided herein by administering to the subject a fusion polypeptide or pharmaceutical composition as described herein, wherein the fusion polypeptide comprises a non-mammalian peptide sequence, the RBD of SARS-CoV-2, and an immune stimulating peptide sequence selected from the group consisting of: immune stimulating peptide sequences 1-4 (as described herein).

### General Definitions

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Features, integers, characteristics, compounds, chemical moieties or groups described in conjunction with a particular aspect, embodiment or example of the invention are to be understood to be applicable to any other aspect, embodiment or example described herein unless incompatible therewith.

As used herein the terms “vaccinate”, “vaccinating”, “vaccination”, “immunization” are taken to include an intervention performed with the intention of reducing risks of getting a disease and/or for lowering the chance of severe disease by working with the body's natural defenses to build protection, i.e., to immunize the body. Immunization is the process of becoming protected against a disease. But it can also mean the same thing as vaccination, which is getting a vaccine to become protected against a disease. This is obtained by administering to the subject a vaccine. A vaccine can be anything known to a person in the art like for example live-attenuated vaccines, inactivated vaccines, subunit, recombinant, polysaccharide, and conjugate vaccines, toxoid vaccines, mRNA vaccines or viral vector vaccines. They can be administered in any form like injections (shots), liquids, pills, or nasal sprays. Vaccination is a preventive treatment in order to obtain protection before and/or after encountering the pathogen.

As used herein, the terms “treat”, “treating” and “treatment” are taken to include an intervention performed with the intention of preventing the development or altering the pathology of a condition, disorder or symptom. Accordingly, “treatment” refers to both therapeutic treatment and prophylactic or preventative measures, wherein the object is to prevent or slow down (lessen) the targeted condition, disorder or symptom.

As used herein, the “administration” or “administering” of a pharmaceutical composition described herein to a subject includes any route of introducing or delivering to a subject which allows for the composition to perform its intended function. Administration can be carried out by any suitable route, including orally, intranasally, intraocularly, ophthalmically, parenterally (intravenously, intramuscularly, intraperitoneally, intradermally or subcutaneously), or topically. Administration includes self-administration and the administration by another. The composition can be administered as a therapeutically effective amount. As used herein, the phrase “therapeutically effective amount” means a dose or plasma concentration in a subject that provides the specific pharmacological effect for which the described compositions are administered, e.g. to treat a disease of interest in a target subject. The therapeutically effective amount may vary based on the route of administration and dosage form, the age and weight of the subject, and/or the disease or condition being treated.

As used herein the term “subject” refers to an individual, e.g., a human, dog, cat, pig, horse, mouse, cow, rat etc having or at risk of having a specified condition, disorder or symptom. The subject may be a patient i.e., a subject in need of treatment/vaccination in accordance with the invention. The subject may have received vaccination/treatment for the condition, disorder or symptom. Alternatively, the subject has not been treated/vaccinated prior to treatment/vaccination in accordance with the present invention. The subject is preferably in need of administration of a fusion polypeptide or composition of the invention.

As used herein the term “non-mammalian peptide sequence” refers to a peptide sequence that does not originate from or are in no way related to mammals. In particular it refers to peptide sequences which are considered foreign to the subject vaccinated or treated with the method or fusion polypeptide as described herein which means, that the immune system of the subject would identify it as non-self and therefore would, if possible, develop antibodies against it. If the fusion polypeptide described herein contains a non-mammalian peptide sequence against which the immune response should be triggered/enhanced by the immune stimulating peptide sequence, the non-mammalian peptide sequence may also be called “peptide (sequence) of interest”.

As used herein the term “mammalian peptide sequence” refers to any sequence relating to or originating from a mammal. Such a peptide sequence would be considered as self/self antigen by the immune system of the subject. If the fusion polypeptide described herein comprises a mammalian peptide sequence against which an immune response should be triggered/enhanced by the immune stimulating peptide sequence, the mammalian peptide sequence may also be called “peptide of interest”.

The term “antigen as a self antigen”, or “self antigen”, as used in the context of the present invention, refers to an antigenic polypeptide moiety. Preferably, such a polypeptide moiety is specific to tumor angiogenesis and not specific to non-tumor related angiogenesis, such as wound healing. These moieties can be conjugated to an immunogen, as described herein, by their full length or a part thereof so as to present or display at least one epitope or antigenic determinant.

The self antigen may be a structural component of a blood vessel formed during tumor angiogenesis, preferably a structural component specific to a blood vessel formed in tumor angiogenesis and not specific to a blood vessel formed in non-tumor related angiogenesis, such as a polypeptide moiety, or a part thereof, selected from the group formed by, or consisting of, tissue inhibitor of metalloproteinase 1 (Timp1), apelin (Apln), serum amyloid A3 (Saa3), CD93 antigen (CD93), heart development protein with EGF-like domains 1 (Heg1), Notch 4, apelin receptor (Aplnr), nestin (Nes), tenascin C (Tnc), C-domain of Tenascin C, pentraxin related gene (Ptx3), vimentin (Vim), prostate specific membrane antigen (PSMA), human EGF receptor-2 (HER2), HER3, tumor necrosis factor alpha induced protein 6 (Tnfaip6), carboxypeptidase Z (Cpz), snail family zinc finger 1 (Snail), premelanosome protein (Pmel), arylsulfatase I (Arsi), WNT inducible signaling pathway protein 1 (Wisp1), glutathione peroxidase 7 (Gpx7), a disintegrin-like and metallopeptidase (reprolysin type) with thrombospondin type 1 motif, 4 (Adamts4), endothelial cell-specific molecule 1 (Esm1), integrin alpha 5 (fibronectin receptor alpha) (Itga5), a disintegrin-like and metallopeptidase (reprolysin type) with thrombospondin type 1 motif, 2 (Adamts2), thrombospondin 2 (Thbs2), matrix metallopeptidase 14 (membrane-inserted) (Mmp14), insulin-like growth factor binding protein 3 (Igfbp3), thrombospondin 1 (Thbsl1, fibrillin 1 (Fbn1), periostin, osteoblast specific factor (Postn), leucine rich repeat containing 17 (Lrrc17), fibrillin 2 (Fbn2), cerebral endothelial cell adhesion molecule (Cercam), secreted frizzled-related protein 4 (Sfrp4), C lq and tumor necrosis factor related protein 6 (C lqtnf6), lysyl oxidase-like 3 (Loxl3), immunoglobulin superfamily, member 10 (Igsf10), secreted frizzled-related protein 2 (Sfrp2), FK506 binding protein 10 (Fkbp10), glutamine fructose-6-phosphate transaminase 2 (Gfpt2), carboxypeptidase X 1 (Cpxm I), microfibrillar associated protein 5 (Mfap5), epidermal growth factor-containing fibulin-like extracellular matrix protein 2 (Efemp2), nephroblastoma overexpressed gene (Nov), versican (42yn), elastin (Eln), cysteine rich protein 61 (Cyr61), sulfatase 1 (Sulf 1), nidogen 2 (Nid2), CD248 antigen, endosialin (Cd248), lysyl oxidase-like 2 (Loxl2), follistatin-like 1 (Fstl1), von Willebrand factor type A, EGF and pentraxin domain containing 1 (Svep1), laminin, alpha 4 (Lama4), slit homolog 3 (Slit3), mannose receptor, C type 2 (Mrc2), cytoskeleton-associated protein 4 (Ckap4), G protein-coupled receptor 133 (Gpr133), fascin homolog 1, actin bundling protein (Fscn1), elastin microfibril interfacer 2 (Emilin2), scavenger receptor class A, member 3 (Scara3), serine (or cysteine) peptidase inhibitor, clade B, member 2 (Serpinb2), chemokine (C—C motif) ligand 2 (Ccl2), insulin receptor (Insr), folate hydrolase 1 (Folh1), CD99 antigen (CD99), casein kappa (Csn3), calcitonin receptor-like (Calcrl), activin A receptor, type II-like 1 (Acvrl1), colony stimulating factor 3 receptor (Csf3r), chloride channel calcium activated 2 (Clca2), C-type lectin domain family 14, member a (Clec14a), transmembrane protein 100 (Tmem100), cysteine and tyrosine-rich protein 1 (Cyyr1), alkaline ceramidase 2 (Acer2), trichorhinophalangeal syndrome I (TrpsI), ArfGAP with RhoGAP domain, ankyrin repeat and PH domain 3 (Arap3), integrin alpha 8 (Itga8), sema domain, immunoglobulin domain (Ig), short basic domain, secreted, (43ynthesized) 3G (Sema3g), transmembrane protein 2 (Tmem2), tumor necrosis factor (ligand) superfamily, member 10 (Tnfsf10), HOP homeobox (Hopx), lactalbumin, alpha (Lalba), phosphatidylinositol-3,4,5-trisphosphate-dependent Rac exchange factor 2 (Prex2), mucin 15 (Muc15), rhotekin 2 (Rtkn2), SRY (sex determining region Y)-box 4 (Sox4), tetraspanin 18 (Tspan18), G protein-coupled receptor 126 (Gpr126), C-type lectin domain family 1, member a (Clecla), extra domain-B of fibronectin (ED-B), extra domain-A of fibronectin (ED-A) and hairy/enhancer-of-split related with YRPW motif 1 (Hey1) as indicated in Tables 1 and 2 below.

TABLE 1
list of tumour angiogenesis specific markers

| | Gene ID | Gene ID | Gene | |
|---|---|---|---|---|
| # | mouse | human | name | Gene ID |
| 1 | 21857 | 7076 | Timp1 | tissue inhibitor of metalloproteinase 1 |
| 2 | 30878 | 8862 | Apln | apelin |
| 3 | 20210 | 6290 | Saa3 | serum amyloid A3 |
| 4 | 17064 | 22918 | Cd93 | CD93 antigen |
| 5 | 77446 | 57493 | Heg1 | heart development protein with EGF-like domains 1 |
| 6 | 18132 | 4855 | Notch4 | notch 4 [Source: MGI Symbol; Acc: MGI: 107471] |
| 7 | 23796 | 187 | Aplnr | apelin receptor [Source: MGI Symbol; Acc: MGI: 1346086] |
| 8 | 18008 | 10763 | Nes | nestin [Source: MGI Symbol; Acc: MGI: 101784] |
| 9 | 21923 | 3371 | Tnc | tenascin C [Source: MGI Symbol; Acc: MGI: 101922] |
| 10 | 19288 | 5806 | Ptx3 | pentraxin related gene [Source: MGI Symbol; Acc: MGI: 104641] |
| 11 | 22352 | 7431 | Vim | vimentin [Source: MGI Symbol; Acc: MGI: 98932] |
| 12 | 21930 | 7130 | Tnfaip6 | tumor necrosis factor alpha induced protein 6 [Source: MGI Symbol; |
| | | | | Acc: MGI: 1195266] |
| 13 | 242939 | 8532 | Cpz | carboxypeptidase Z [Source: MGI Symbol; Acc: MGI: 88487] |
| 14 | 20613 | 6615 | Snai1 | snail family zinc finger 1 |
| 15 | 20431 | 6490 | Pmel | premelanosome protein [Source: MGI Symbol; Acc: MGI: 98301] |
| 16 | 545260 | 340075 | Arsi | arylsulfatase I [Source: MGI Symbol; Acc: MGI: 2670959] |
| 17 | 22402 | 8840 | Wisp1 | WNT1 inducible signaling pathway protein 1 [Source: MGI Symbol; |
| | | | | Acc: MGI: 1197008] |
| 18 | 67305 | 2882 | Gpx7 | glutathione peroxidase 7 [Source: MGI Symbol; Acc: MGI: 1914555] |
| 19 | 240913 | 9507 | Adamts4 | a disintegrin-like and metallopeptidase (reprolysin type) with |
| | | | | thrombospondin type 1 motif, 4 [Source: MGI Symbol; |
| | | | | Acc: MGI: 1339949] |
| 20 | 71690 | 11082 | Esm1 | endothelial cell-specific molecule 1 [Source: MGI Symbol; |
| | | | | Acc: MGI: 1918940] |
| 21 | 16402 | 3678 | Itga5 | integrin alpha 5 (fibronectin receptor alpha) [Source: MGI Symbol; |
| | | | | Acc: MGI: 96604] |
| 22 | 216725 | 9509 | Adamts2 | a disintegrin-like and metallopeptidase (reprolysin type) with |
| | | | | thrombospondin type 1 motif, 2 [Source: MGI Symbol; |
| | | | | Acc: MGI: 1347356] |
| 23 | 21826 | 7058 | Thbs2 | thrombospondin 2 [Source: MGI Symbol; Acc: MGI: 98738] |
| 24 | 17387 | 4323 | Mmp14 | matrix metallopeptidase 14 (membrane-inserted) [Source: MGI Symbol; |
| | | | | Acc: MGI: 101900] |
| 25 | 16009 | 3486 | Igfbp3 | insulin-like growth factor binding protein 3 [Source: MGI Symbol; |
| | | | | Acc: MGI: 96438] |
| 26 | 21825 | 7057 | Thbs1 | thrombospondin 1 [Source: MGI Symbol; Acc: MGI: 98737] |
| 27 | 14118 | 2200 | Fbn1 | fibrillin 1 [Source: MGI Symbol; Acc: MGI: 95489] |
| 28 | 50706 | 10631 | Postn | periostin, osteoblast specific factor [Source: MGI Symbol; |
| | | | | Acc: MGI: 1926321] |
| 29 | 74511 | 10234 | Lrrc17 | leucine rich repeat containing 17 [Source: MGI Symbol; |
| | | | | Acc: MGI: 1921761] |
| 30 | 14119 | 2201 | Fbn2 | fibrillin 2 [Source: MGI Symbol; Acc: MGI: 95490] |
| 31 | 99151 | 51148 | Cercam | cerebral endothelial cell adhesion molecule [Source: MGI Symbol; |
| | | | | Acc: MGI: 2139134] |
| 32 | 20379 | 6424 | Sfrp4 | secreted frizzled-related protein 4 [Source: MGI Symbol; |
| | | | | Acc: MGI: 892010] |
| 33 | 72709 | 114904 | C1qtnf6 | C1q and tumor necrosis factor related protein 6 [Source: MGI |
| | | | | Symbol; Acc: MGI: 1919959] |
| 34 | 16950 | 84695 | Loxl3 | lysyl oxidase-like 3 [Source: MGI Symbol; Acc: MGI: 1337004], |
| | | | | also known as Loxl2 |
| 35 | 242050 | 285313 | Igsf10 | immunoglobulin superfamily, member 10 [Source: MGI Symbol; |
| | | | | Acc: MGI: 1923481] |
| 36 | 20319 | 6423 | Sfrp2 | secreted frizzled-related protein 2 [Source: MGI Symbol; |
| | | | | Acc: MGI: 108078] |
| 37 | 14230 | 60681 | Fkbp10 | FK506 binding protein 10 [Source: MGI Symbol; Acc: MGI: 104769] |
| 38 | 14584 | 9945 | Gfpt2 | glutamine fructose-6-phosphate transaminase 2 [Source: MGI |
| | | | | Symbol; Acc: MGI: 1338883] |
| 39 | 56264 | 56265 | Cpxm1 | carboxypeptidase X 1 (M14 family) [Source: MGI Symbol; |
| | | | | Acc: MGI: 1934569] |
| 40 | 50530 | 8076 | Mfap5 | microfibrillar associated protein 5 [Source: MGI Symbol; |
| | | | | Acc: MGI: 1354387] |
| 41 | 58859 | 30008 | Efemp2 | epidermal growth factor-containing fibulin-like extracellular |
| | | | | matrix protein 2 [Source: MGI Symbol; Acc: MGI: 1891209] |
| 42 | 18133 | 4856 | Nov | nephroblastoma overexpressed gene [Source: MGI Symbol; |
| | | | | Acc: MGI: 109185] |
| 43 | 13003 | 1462 | Vcan | versican [Source: MGI Symbol; Acc: MGI: 102889] |
| 44 | 13717 | 2006 | Eln | elastin [Source: MGI Symbol; Acc: MGI: 95317] |
| 45 | 16007 | 3491 | Cyr61 | cysteine rich protein 61 [Source: MGI Symbol; Acc: MGI: 88613] |
| 46 | 240725 | 23213 | Sulf1 | sulfatase 1 [Source: MGI Symbol; Acc: MGI: 2138563] |
| 47 | 18074 | 22795 | Nid2 | nidogen 2 [Source: MGI Symbol; Acc: MGI: 1298229] |
| 48 | 70445 | 57124 | Cd248 | CD248 antigen, endosialin [Source: MGI Symbol; |
| | | | | Acc: MGI: 1917695] |
| 49 | 94352 | 4017 | Loxl2 | lysyl oxidase-like 2 [Source: MGI Symbol; Acc: MGI: 2137913] |
| 50 | 14314 | 11167 | Fstl1 | follistatin-like 1 [Source: MGI Symbol; Acc: MGI: 102793] |
| 51 | 64817 | 79987 | Svep1 | sushi, von Willebrand factor type A, EGF and pentraxin domain |
| | | | | containing 1 [Source: MGI Symbol; Acc: MGI: 1928849] |
| 52 | 16775 | 3910 | Lama4 | laminin, alpha 4 [Source: MGI Symbol; Acc: MGI: 109321] |
| 53 | 20564 | 6586 | Slit3 | slit homolog 3 (Drosophila) [Source: MGI Symbol; |
| | | | | Acc: MGI: 1315202] |
| 54 | 17534 | 9902 | Mrc2 | mannose receptor, C type 2 [Source: MGI Symbol; |
| | | | | Acc: MGI: 107818] |
| 55 | 216197 | 10970 | Ckap4 | cytoskeleton-associated protein 4 [Source: MGI Symbol; |
| | | | | Acc: MGI: 2444926] |
| 56 | 243277 | 283383 | Gpr133 | G protein-coupled receptor 133 [Source: MGI Symbol; |
| | | | | Acc: MGI: 3041203] |
| 57 | 14086 | 6624 | Fscn1 | fascin homolog 1, actin bundling protein (Strongylocentrotus |
| | | | | purpuratus) [Source: MGI Symbol; Acc: MGI: 1352745] |
| 58 | 246707 | 84034 | Emilin2 | elastin microfibril interfacer 2 [Source: MGI Symbol; |
| | | | | Acc: MGI: 2389136] |
| 59 | 219151 | 51435 | Scara3 | scavenger receptor class A, member 3 [Source: MGI Symbol; |
| | | | | Acc: MGI: 2444418] |
| 60 | 18788 | 5055 | Serpinb2 | serine (or cysteine) peptidase inhibitor, clade B, member 2 |
| | | | | [Source: MGI Symbol; Acc: MGI: 97609] |
| 61 | 20296 | 6347 | Ccl2 | chemokine (C-C motif) ligand 2 [Source: MGI Symbol; |
| | | | | Acc: MGI: 98259] |
| 62 | 16337 | 3643 | Insr | insulin receptor [Source: MGI Symbol; Acc: MGI: 96575]; |
| | | | | D630014A15Rik, CD220, IR-B, 4932439J01Rik, IR-A, IR |
| 63 | 53320 | 219595 | Folh1 | folate hydrolase 1 [Source: MGI Symbol; Acc: MGI: 1858193]; |
| | | | | prostate-specific membrane antigen, glutamate carboxypeptidase II |
| 64 | 673094 | 4267 | CD99 | CD99 antigen |
| 65 | 12994 | 1448 | Csn3 | casein kappa [Source: MGI Symbol; Acc: MGI: 107461] |
| 66 | 54598 | 10203 | Calcrl | calcitonin receptor-like [Source: MGI Symbol; Acc: MGI: 1926944] |
| 67 | 11482 | 94 | Acvrl1 | activin A receptor, type II-like 1 [Source: MGI Symbol; |
| | | | | Acc: MGI: 1338946], ALK1 |
| 68 | 12986 | 1441 | Csf3r | colony stimulating factor 3 receptor (granulocyte) [Source: MGI |
| | | | | Symbol; Acc: MGI: 1339755] |
| 69 | 80797 | | Clca2 | chloride channel calcium activated 2 [Source: MGI Symbol; |
| | | | | Acc: MGI: 1931471] |
| 70 | 66864 | 161198 | Clec14a | C-type lectin domain family 14, member a [Source: MGI Symbol; |
| | | | | Acc: MGI: 1914114] |
| 71 | 67888 | 55273 | Tmem100 | transmembrane protein 100 [Source: MGI Symbol; |
| | | | | Acc: MGI: 1915138] |
| 72 | 224405 | 116159 | Cyyr1 | cysteine and tyrosine-rich protein 1 [Source: MGI Symbol; |
| | | | | Acc: MGI: 2152187] |
| 73 | 13717 | 2006 | Eln | elastin [Source: MGI Symbol; Acc: MGI: 95317] |
| 74 | 230379 | 340485 | Acer2 | alkaline ceramidase 2 [Source: MGI Symbol; Acc: MGI: 1920932] |
| 75 | 83925 | 7227 | Trps1 | trichorhinophalangeal syndrome I (human) [Source: MGI Symbol; |
| | | | | Acc: MGI: 1927616] |
| 76 | 106952 | 64411 | Arap3 | ArfGAP with RhoGAP domain, ankyrin repeat and PH domain 3 |
| | | | | [Source: MGI Symbol; Acc: MGI: 2147274] |
| 77 | 241226 | 8516 | Itga8 | integrin alpha 8 [Source: MGI Symbol; Acc: MGI: 109442] |
| 78 | 218877 | 56920 | Sema3g | sema domain, immunoglobulin domain (Ig), short basic domain, |
| | | | | secreted, (48ynthesized) 3G [Source: MGI Symbol; |
| | | | | Acc: MGI: 3041242] |
| 79 | 83921 | 23670 | Tmem2 | transmembrane protein 2 [Source: MGI Symbol; Acc: MGI: 1890373] |
| 80 | 22035 | 8743 | Tnfsf10 | tumor necrosis factor (ligand) superfamily, member 10 |
| | | | | [Source: MGI Symbol; Acc: MGI: 107414] |
| 81 | 74318 | 84525 | Hopx | HOP homeobox [Source: MGI Symbol; Acc: MGI: 1916782] |
| 82 | 16770 | 3906 | Lalba | lactalbumin, alpha [Source: MGI Symbol; Acc: MGI: 96742] |
| 83 | 109294 | 80243 | Prex2 | phosphatidylinositol-3,4,5-trisphosphate-dependent Rac exchange |
| | | | | factor 2 [Source: MGI Symbol; Acc: MGI: 1923385] |
| 84 | 269328 | 143662 | Muc15 | mucin 15 [Source: MGI Symbol; Acc: MGI: 2442110] |
| 85 | 170799 | 219790 | Rtkn2 | rhotekin 2 [Source: MGI Symbol; Acc: MGI: 2158417] |
| 86 | 20677 | 6659 | Sox4 | SRY (sex determining region Y)-box 4 [Source: MGI Symbol; |
| | | | | Acc: MGI: 98366] |
| 87 | 241556 | 90139 | Tspan18 | tetraspanin 18 [Source: MGI Symbol; Acc: MGI: 1917186] |
| 88 | 215798 | 57211 | Gpr126 | G protein-coupled receptor 126 [Source: MGI Symbol; |
| | | | | Acc: MGI: 1916151] |
| 89 | 243653 | 51267 | Clec1a | C-type lectin domain family 1, member a [Source: MGI Symbol; |
| | | | | Acc: MGI: 2444151] |
| 90 | 15213 | 23462 | Hey1 | hairy/enhancer-of-split related with YRPW motif 1 [Source: MGI |
| | | | | Symbol; Acc: MGI: 1341800] |

TABLE 2
Tumour angiogenesis specific markers coupled to mRNA/protein Genbank Acc. No.'s

| # | GenBank Acc. No. mRNA/protein mouse | GenBank Acc. No. mRNA/protein human |
|---|---|---|
| 1 | NM_001044384.1 | NM_003254.2 |
| 2 | NM_013912.3 | NM_017413.4 |
| 3 | NM_011315.3 | S73444.1 |
| 4 | NP_034870.1, NM_010740.3 | NP_036204.2, NM_012072.3 |
| 5 | NP_780465.4, NM_175256.5 | NP_065784.1, NM_020733.1 |
| 6 | NP_035059.2, NM_010929.2 | NP_004548.3, NM_004557.3 |
| 7 | NP_035914.1, NM_011784.3 | NP_005152.1, NM_005161.4, NR_027991.1 |
| 8 | NP_057910.3, NM_016701.3 | NP_006608.1, NM_006617.1 |
| 9 | NP_035737.2, NM_011607.3 | NP_002151.2, NM_002160.3 |
| 10 | NP_033013.3, NM_008987.3 | NP_002843.2, NM_002852.3 |
| 11 | NP_035831.2, NM_011701.4 | NP_003371.2, NM_003380.3 |
| 12 | NP_033424.1, NM_009398.2 | NP_009046.2, NM_007115.3 |
| 13 | NP_694747.2, NM_153107.2 | NP_003643.2, NM_003652.3, NP_001014447.1, |
| | | NM_001014447.2, NP_001014448.1, NM_001014448.2 |
| 14 | NP_035557.1, NM_011427.2 | NP_005976.2, NM_005985.3 |
| 15 | NP_068682.2, NM_021882.4, | NP_001186983.1, NM_001200054.1, |
| | XP_006513468.1, XM_006513405.2 | NP_001186982.1, NM_001200053.1 |
| 16 | NP_001033588.1, NM_001038499.1 | NP_001012301.1, NM_001012301.2 |
| 17 | NP_061353.1, NM_018865.2 | NP_001191799.1, NM_001204870.1 |
| 18 | NP_077160.1, NM_024198.3 | NM_015696.4 |
| 19 | NM_172845.2 | NP_005090.3, NM_005099.4 |
| 20 | NP_076101.1, NM_023612.3 | NP_008967.1, NM_007036.4, NP_001129076.1, |
| | | NM_001135604.1 |
| 21 | NP_034707.3, NM_010577.3 | NP_002196.2, NM_002205.2 |
| 22 | NP_001264234.1, NM_001277305.1, | NP_067610.1, NM_021599.2, NP_055059.2, |
| | NP_783574.1, NM_175643.3 | NM_014244.4 |
| 23 | NP_035711.2, NM_011581.3 | NP_003238.2, NM_003247.3 |
| 24 | NP_032634.3, NM_008608.3 | NP_004986.1, NM_004995.3 |
| 25 | NP_032369.2, NM_008343.2 | NP_001013416.1, NM_001013398.1, NP_000589.2, |
| | | NM_000598.4 |
| 26 | NP_035710.2, NM_011580.3 | NP_003237.2, NM_003246.2 |
| 27 | NP_032019.2, NM_007993.2 | NP_000129.3, NM_000138.4 |
| 28 | NP_001185695.1, NM_001198766.1 | NP_001273596.1, NM_001286667.1 |
| 29 | NP_083253.1, NM_028977.1 | NP_005815.2, NM_005824.2 |
| 30 | NP_034311.2, NM_010181.2 | NP_001990.2, NM_001999.3 |
| 31 | NP_997181.1, NM_207298.2 | NM_001286760.1, NP_001273689.1 |
| 32 | NP_057896.1, NM_016687.3 | NP_003005.2, NM_003014.3 |
| 33 | NM_001204153.1, NP_001191082.1 | NM_031910.3, NP_114116.3 |
| 34 | NP_038614.2, NM_013586.4 | NP_001276093.1, NM_001289164.1 |
| 35 | NP_001156356.1, NM_001162884.1 | NM_001178145.1, NP_001171616.1 |
| 36 | NP_033170.1, NM_009144.2 | NP_003004.1, NM_003013.2 |
| 37 | NP_038557.1, NM_013529.3 | NM_021939.3, NP_068758.3 |
| 38 | NP_038557.1, NM_013529.3 | NP_005101.1, NM_005110.2 |
| 39 | NP_062670.2, NM_019696.2 | NP_001171628.1, NM_001184699.1 |
| 40 | NM_015776.2, NP_056591.1 | NP_001284639.1, NM_001297710.1 |
| 41 | NM_001164352.1, NP_001157824.1 | NP_058634.4, NM_016938.4 |
| 42 | NP_035060.1, NM_010930.4 | NP_002505.1, NM_002514.3 |
| 43 | NP_001127947.1, NM_001134475.1 | NP_004376.2, NM_004385.4 |
| 44 | NP_031951.2, NM_007925.3 | NP_001075223.1, NM_001081754.2 |
| 45 | NP_034646.1, NM_010516.2 | NP_001545.2, NM_001554.4 |
| 46 | NP_001185494.1, NM_001198565.1 | NP_055985.2, NM_015170.2 |
| 47 | NP_032721.2, NM_008695.2 | NP_031387.3, NM_007361.3 |
| 48 | NP_473383.1, NM_054042.2 | NP_065137.1, NM_020404.2 |
| 49 | NP_201582.2, NM_033325.2 | |
| 50 | NP_032073.2, NM_008047.5 | NP_009016.1, NM_007085.4 |
| 51 | NP_073725.2, NM_022814.2 | NP_699197.3, NM_153366.3 |
| 52 | NP_034811.2, NM_010681.4 | NP_002281.3, NM_002290.4 |
| 53 | NP_035542.2, NM_011412.3 | NP_003053.1, NM_003062.3 |
| 54 | NP_032652.3, NM_008626.3 | NP_006030.2, NM_006039.4 |
| 55 | NP_780660.1, NM_175451.1 | NP_006816.2, NM_006825.3 |
| 56 | NP_001074811.1, NM_001081342.1 | NP_942122.2, NM_198827.3 |
| 57 | NP_032010.2, NM_007984.2 | NP_003079.1, NM_003088.3 |
| 58 | NP_660140.1, NM_145158.3 | NP_114437.2, NM_032048.2 |
| 59 | NP_766192.1, NM_172604.3 | NP_878185.1, NM_182826.1 |
| 60 | NM_001174170.1, NP_001167641.1 | NP_001137290.1, NM_001143818.1 |
| 61 | NP_035463.1, NM_011333.3 | NP_002973.1, NM_002982.3 |
| 62 | NP_034698.2, NM_010568.2 | NP_001073285.1, NM_001079817.1 |
| 63 | NP_001153178.1, NM_001159706.1 | NP_710163.1, NM_153696.2 |
| 64 | NP_079860.2, NM_025584.2 | NP_001264639.1, NM_001277710.1 |
| 65 | NP_031812.2, NM_007786.4 | NP_005203.2, NM_005212.2 |
| 66 | NP_061252.2, NM_018782.2 | NP_001258680.1, NM_001271751.1 |
| 67 | NP_001264188.1, NM_001277259.1 | NP_000011.2, NM_000020.2 |
| 68 | NP_031808.2, NM_007782.3 | NP_758519.1, NM_172313.2 |
| 69 | NP_085104.1, NM_030601.3 | |
| 70 | NP_080085.3, NM_025809.5 | NP_778230.1, NM_175060.2 |
| 71 | NP_080709.1, NM_026433.2 | NP_001093110.1, NM_001099640.1 |
| 72 | NP_659102.1, NM_144853.3 | NP_443186.1, NM_052954.2 |
| 73 | NP_031951.2, NM_007925.3 | NP_001075223.1, NM_001081754.2 |
| 74 | NP_001277472.1, NM_001290543.1 | NP_001010887.2, NM_001010887.2 |
| 75 | NP_114389.2, NM_032000.2 | NP_054831.2, NM_014112.4 |
| 76 | NP_001192265.1, NM_001205336.1 | NP_071926.4, NM_022481.5 |
| 77 | NP_001001309.1, NM_001001309.2 | NP_001278423.1, NM_001291494.1 |
| 78 | NP_001020550.1, NM_001025379.1 | NP_064548.1, NM_020163.1 |
| 79 | NP_001028931.1, NM_001033759.2 | NP_001129292.1, NM_001135820.1 |
| 80 | NP_033451.1, NM_009425.2 | NP_001177871.1, NM_001190942.1 |
| 81 | NP_783199.1, NM_175606.3 | NP_001138932.1, NM_001145460.1 |
| 82 | NP_034809.1, NM_010679.1 | NP_002280.1, NM_002289.2 |
| 83 | NP_083801.1, NM_029525.1 | NP_079146.2, NM_024870.2 |
| 84 | NP_766567.1, NM_172979.3 | NP_001128564.1, NM_001135092.1 |
| 85 | NP_001074815.1, NM_001081346.1 | NP_660350.2, NM_145307.3 |
| 86 | NP_033264.2, NM_009238.2 | NP_003098.1, NM_003107.2 |
| 87 | NP_899003.1, NM_183180.2 | NP_570139.3, NM_130783.4 |
| 88 | NP_001002268.1, NM_001002268.3 | NP_065188.4, NM_020455.5 |
| 89 | NP_780735.2, NM_175526.3 | NP_001284678.1, NM_001297749.1 |
| 90 | NP_034553.2, NM_010423.2 | NP_036390.3, NM_012258.3 |

More preferably, the polypeptide moieties are specific to (i) embryonic tissue and (ii) blood vessels formed in tumor angiogenesis, or (iii) tumor cells. Even more preferably, the self antigen is selected from the group formed by or consisting of vimentin (Vim), CD93, CD99, HER2, HER3, prostate specific membrane antigen (PSMA). Based on the screening method as described herein, the skilled person is able to identify further self antigens that fulfill the aforementioned criteria.

In an alternatively preferred embodiment, the self antigen is a specific disease antigen selected from Vim and CD99. These self antigens have an anti-tumor effect.

In another alternatively preferred embodiment, the self antigen is CD93 antigen. This self antigen is high in METs and especially beneficial in aspects of this invention.

In yet another alternatively preferred embodiment, the self antigen is a specific disease antigen selected from the group consisting of Mmp 14, Cercam, Sulf1, Gpr133, Scara3, Insr, Folh1, CD99, Acvrl1, Itga8, Muc15. These self antigens are membrane antigens.

In yet another alternatively preferred embodiment of aspects of this invention, the self antigen is a specific disease antigen selected from the group consisting of Timp1, Apln, Saa3, Ptx3, Tnfaip6, Cpz, Pmel, Arsi, Adamts4, Thbs2, Mmp14, Igfbp3, Thbs1, Fbn1, Postn, Lox13, Mfap5, Efemp2, Nov, 55yn, Eln, Cyr61, Sulf1, Nid2, Lama4, Slit3, Mrc2, Serpinb2, Ccl2, CD99, Tnfsf10, Lalba, Vim. These self antigens are secreted antigens.

In yet another alternatively preferred embodiment, the self antigen is a specific disease antigen selected from the group consisting of Apln, Saa3, Vim, Pmel, Arsi, Wisp1, Fkbp10. These self antigens are cytoplasmatic targets.

In yet another alternatively preferred embodiment of aspects of this invention, the self antigen is a specific disease antigen selected from the group consisting of Timp1, Apln, Saa3, Ptx3, Vim, Tnfaip6, Cpz, Arsi, Esml, Itga5, Aclamts2, Thbs2, Mmp14, Thbs1, Fbn1, Postn, Fbn2, Cercam, Lox13, Mfap5, Efemp2, Nov, 55yn, Eln, Cyr61, Sulf1, Nid2, Lama4, Slit3, Mrc2, Gpr133, Scara3, Insr, Folh1, CD99, Acvrl1, Eln, Itga8, Tnfsf10, Lalba, Muc15. These self antigens are extracellular targets.

In yet another alternatively preferred embodiment of aspects of this invention, the self antigen is a specific disease antigen selected from the group consisting of Vim, Cd93, Heg1, Notch4, Aplnr, Nes, Tnc, Snail, Gpx7, Lrrc17, Sfrp4, Clqtnf6, Igsf10, Sfrp2, Gfpt2, Cpxm1, Cd248, Lox12, Fstl1, Svep1, Ckap4, Emilin2, Csn3, Calcrl, Csf3r, Clca2, Clec14a, Tmem100, Cyyr1, Acer2, Trps1, Arap3, Sema3g, Tmem2, Hopx, Prex2, Rtkn2, Sox4, Tspan18, Gpr126, Clecla, Hey1. These self antigens are involved in various disease related processes.

In yet another alternatively preferred embodiment of aspects of this invention, the self antigen is a specific disease antigen selected from the group consisting of Vim, Timp1, Apln, Saa3, Cd93, Heg1, Notch4, Aplnr, Nes, Tnc, Ptx3, Vim, Tnfaip6, Cpz, Snail, Pmel, Arsi, Wisp 1, Gpx7, Adamts4, Esm1, Itga5, Adamts2, Thbs2, Mmp14, Igfbp3, Thbs1, Fbn1, Postn, Lrrc17, FFbn2, Cercam, Sfrp4, Clqtnf6, Lox13, Igsf10, Sfrp2, Fkbp10, Gfpt2, Cpxm1, Mfap5, Efemp2, Nov, 56yn, Eln, Cyr61, Sulf1, Nid2, Cd248, Lox12, Fstl1, Svep1, Lama4, Slit3, Mrc2, Ckap4, Gpr133, Fscn1, Emilin2, Scara3, Serpinb2, Ccl2, Insr, Folh1, CD99. These self antigens are involved in tumor development.

In yet another alternatively preferred embodiment of aspects of this invention, the self antigen is a specific disease antigen selected from the group consisting of vim, Csn3, Calcrl, Acvrl1, Csf3r, Clca2, Clec14a, Tmem100, Cyyr1, Eln, Acer2, Trps1, Arap3, Itga8, Sema3g, Tmem2, Tnfsf10, Hopx, Lalba, Prex2, Muc15, Rtkn2, Sox4, Tspan18, Gpr126, Clec1a, Hey1. These self antigens are involved in metastasis.

Preferably, the self antigen is extra domain-B of fibronectin or vimentin (also referred to as Vim herein). More preferably, the self antigen is human extra domain-B of fibronectin or human vimentin.

The term “blood vessel”, as used herein, may refer to part of the vascular system comprised of endothelial cells, extracellular matrix, a basal membrane.

The term “tumor vasculature”, as used herein, refers to a system of blood vessels, recruited during tumor angiogenesis, securing a blood supply to the tumor. One skilled in the art can identify tumor vasculature on the basis of blood vessel structure parameters such as extracellular matrix composition and/or vascular permeability by using (electron)microscopy.

As used herein the term “immune stimulating peptide sequence” refers to a peptide sequence that can be considered antigenic, immunogenic, immune activating etc., whereby all these terms are used interchangeably.

These terms, as used herein, refer to the ability of a (poly)peptide, molecule, compound, for example formulated in a vaccine, to elicit an immune response, either humoral or cell-mediated, or both. If an antigen/peptide sequence elicits such an immune response it can also be referred to as an immunogen. Preferably, the immune response is a humoral immune response mediated primarily by B cells and helper T cells. The skilled person is well aware of standard tests for measuring immunogenicity. It is well established that a T-cell response can be determined with ELISPOT or ELISA, and that a humoral immune response can be determined with ELISA.

The terms “trigger an Immune response”, “elicit an Immune response”, “stimulate the Immune system” and “activate the immune system” as used herein are used interchangeably and refer to the activation of the immune system to respond to an antigen/peptide by a humoral and/or cell-mediated immune response. The terms “enhance”, “increase”, or “boost” as used herein in this context refer to an already existing immune response that is intensified.

Herein the terms “diseases”, “disorders”, and “conditions” are used interchangeably and refer to a disorder of structure or function in a human or animal, especially one that produces specific symptoms or that affects a specific location and is not simply a direct result of physical injury.

Diseases that can be treated and/or prevented with the compositions described herein can be infectious diseases and diseases in which angiogenesis is involved. Examples of diseases that can be treated and/or prevented with the compositions described herein are arthritis (e.g. rheumatoid arthritis), atherosclerosis, restenosis, transplant arteriopathy, warts, scar keloids, synovitis, osteomyelitis, asthma, nasal polyps, polypoidal choroidal vasculopathy, age-related macular degeneration, retinopathy of prematurity, diabetic retinopathy, AIDS, IBD, Crohn's disease, endometriosis, uterine bleeding, psoriasis, myoma's, cancer, or combinations thereof.

“Infectious diseases” are diseases caused by the entrance into the body of pathogenic agents or microorganisms (such as bacteria, viruses, protozoans, parasites, yeast, mycobacteria or fungi) which grow and multiply there. Examples of infectious diseases that can be treated and or prevented with the compositions described herein are influenza and corona virus disease (2019).

Cancers treatable using the fusion polypeptides and compositions of the invention include carcinomas, sarcomas, leukaemias, lymphomas, and other types of cancer.

The term “tumor”, as used herein, refers to a cellular mass exhibiting abnormal growth in tissue, which occurs when cellular proliferation is more rapid than proliferation of normal tissue and continues to grow after the stimuli that normally initiate growth cease. As used herein the term “tumor” includes cancer cells, necrosis, as well as stroma. Tumors generally exhibit partial or complete lack of structural organization and functional coordination with normal, healthy tissue, and usually form a distinct mass of tissue which may be benign or malignant. Preferably, the tumor treated or vaccinated against is a malignant tumor requiring tumor angiogenesis in order to continue growing and eventually metastasizing. More preferably, said tumor is a leukemia or a solid tumor or solid cancer, preferably selected from the group formed by sarcomas, carcinomas, and lymphomas. Exemplary solid tumors include but are not limited to sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, osteosarcoma, hemangiosarcoma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatocellular carcinoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioblastoma multiforme, astrocytoma, medulloblastoma, craniopharyngioma, ependymoma, pinealoma, hemangioblastoma, acoustic neuroma, oligodendroglioma, melanoma, neuroblastoma, and retinoblastoma.

The term “leukaemia” refers broadly to progressive, malignant diseases of the blood-forming organs and is generally characterized by a distorted proliferation and development of leukocytes and their precursors in the blood and bone marrow. Leukaemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukaemic or aleukaemic (subleukaemic). Accordingly, the present invention includes a method of treating leukaemia, and, preferably, a method of treating acute nonlymphocytic leukaemia, chronic lymphocytic leukaemia, acute granulocytic leukaemia, chronic granulocytic leukaemia, acute promyelocyte leukaemia, adult T-cell leukaemia, aleukaemic leukaemia, a leukocythemic leukaemia, 58 ynthesize leukaemia, blast cell leukaemia, bovine leukaemia, chronic myelocytic leukaemia, leukaemia cutis, embryonal leukaemia, eosinophihc leukaemia, Gross' leukaemia, hairy-cell leukaemia, hemoblastic leukaemia, hemocytoblastic leukaemia, histiocytic leukaemia, stem cell leukaemia, acute monocytic leukaemia, leukopenic leukaemia, lymphatic leukaemia, lymphoblastic leukaemia, lymphocytic leukaemia, lymphogenous leukaemia, lymphoid leukaemia, lymphosarcoma cell leukaemia, mast cell leukaemia, megakaryocyte leukaemia, micromyeloblastic leukaemia, monocytic leukaemia, myeloblasts leukaemia, myelocytic leukaemia, myeloid granulocytic leukaemia, myelomonocytic leukaemia, Naegeh leukaemia, plasma cell leukaemia, plasmacytic leukaemia, promyelocyte leukaemia, Rieder cell leukaemia, Schilling's leukaemia, stem cell leukaemia, subleukaemic leukaemia, and undifferentiated cell leukaemia.

The terms “antigen” and “epitope” as used herein are well known to a person skilled in the art and although the epitope corresponds the (small molecular) part of the antigen against which the immune system will produce antibodies the terms may be used interchangeably herein (unless the context does not allow it) since they both represent a part to which the immune system will react to. Both originate from a pathogen against which protection is desired.

Conservative variants will typically contain only conservative substitutions of one or more amino acids of SEQ ID NO: 1, or a substitution, deletion or insertion of non-critical amino acids in non-critical regions of the protein. A conservative variant of SEQ ID NO: 1 may therefore be a conservative amino acid sequence wherein of SEQ ID NO: 1, wherein the variant still comprises those parts of the amino acid sequence that are necessary to obtain antibodies directed against them that in the end will provide protection against a SARS-CoV-2 infection.

Conservative substitutions are well known to a person skilled in the art and also encompass variants wherein amino acids with similar characteristic are replaced by each other. For example, leucine and isoleucine are both aliphatic, branched hydrophobes. Similarly, aspartic acid and glutamic acid are both small, negatively charged residues. Based on their structure and general chemical characteristics of their side chains amino acids can be sorted into the following six groups:
Aliphatic (Glycine, Alanine, Valine, Leucine, Isoleucine)
Hydroxyl or sulfur/selenium-containing (Serine, Cysteine, Selenocysteine, Threonine, Methionine)
Cyclic (Proline)
Aromatic (Phenylalanine, Tyrosine, Tryptophan)
Basic (Histidine, Lysine, Arginine)
Acidic and their amides (Aspartate, Glutamate, Asparagine, Glutamine)

Replacements of amino acids by amino acids of the same group can be considered conservative.

Calculations of sequence homology or identity (the terms are used interchangeably herein) between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970, J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

Alternatively, the percent identity between two amino acid or nucleotide sequences can be determined using the algorithm of Meyers et al. (1989, CABIOS 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990, J. Mol. Biol. 215:403-410). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used. See <http://www.ncbi.nlm.nih.gov>.

The polypeptides described herein can have amino acid sequences sufficiently or substantially identical to the amino acid sequences of SEQ ID NO:1 to 22. The terms “sufficiently identical” or “substantially identical” are used herein to refer to a first amino acid or nucleotide sequence that contains a sufficient or minimum number of identical or equivalent (e.g. with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical.

As used herein, “pharmaceutically acceptable” refers to a material that is not biologically or otherwise undesirable, i.e., the material may be administered to an individual along with the selected neurotoxin without causing any undesirable biological effects or interacting in a deleterious manner with any of the other components of the pharmaceutical composition in which it is contained.

Excipients are natural or synthetic substances formulated alongside an active ingredient (e.g. a neurotoxin as provided herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art and include cell-penetrating peptides. A suitable adjuvant is therefore easily identifiable by one of ordinary skill in the art.

Diluents are diluting agents. Pharmaceutically acceptable diluents are well known in the art and include water or saline. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art and include serum albumin. A suitable carrier is therefore easily identifiable by one of ordinary skill in the art.

The term “nucleic acid” as used he″ein ′ypically refers to an oligomer or polymer (preferably) a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or 62ynthesized bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term nucleic acid further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically 62ynthesized) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically 62ynthesized. A nucleic acid can be double-stranded, partly double stranded, or single-stranded. In adenoviruses the linear nucleic acid that forms the viral genome typically have polypeptides derived from the precursor of the terminal protein (pTP) covalently coupled to each of the 5′ ends of the polynucleotide chain. Where single-stranded, the nucleic acid can be the sense strand or the antisense strand. In addition, nucleic acid can be circular or linear. The terms “polynucleotide” and “oligonucleotide” are used interchangeably and refer to a polymeric form of nucleotides of any length, either deoxyribonucleotides or ribonucleotides or analogs thereof.

The term “vector” Is well known In the art, and as used herein refers to a nucleic acid molecule, e.g. double-stranded DNA. In one example, the vector has an exogenous nucleic acid sequence inserted into it. A vector can suitably be used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a suitable host cell, the vector can replicate independently of, or coincidental with, the host chromosomal DNA; several copies of the vector and its inserted nucleic acid molecule may be generated. Vectors of the present invention can be episomal vectors (i.e., that do not integrate into the genome of a host cell) or can be vectors that integrate into the host cell genome. This definition includes both non-viral and viral vectors. Non-viral vectors include but are not limited to plasmid vectors (e.g. pMA-RQ, pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Larger vectors such as artificial chromosomes (bacteria (BAC), yeast (YAC), or human (HAC)) may be used to accommodate larger inserts. In one particular example, a vector described herein may therefore be a plasmid vector. Such plasmid vectors may be present within a cell. In one example, therefore a cell may be provided which comprises a vector (e.g. a plasmid as described herein) comprising a nucleic acid sequence described herein. A cell may therefore be provided comprising a nucleic acid sequence of the invention.

An expression vector of the Invention is preferably suited for insertion of a nucleic acid molecule of the invention into a prokaryotic cell including a bacterial cell, or, more preferred, into a eukaryotic host cell, such as a yeast cell and a mammalian cell. Particularly preferred is the expression vector pET-21a or pCMV4. Further expression vectors compatible with the present invention are TOPO, CMV or pcDNA 3.1.

A vector as defined herein may also be a viral vector. A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors (AAV), alphavirus vectors and the like. Typically, but not necessarily, viral vectors are replication-deficient as they have lost the ability to propagate in a given cell since viral genes essential for replication have been eliminated from the viral vector. However, some viral vectors can also be adapted to replicate specifically or preferentially in a given cell, such as e.g. a cancer cell, and are typically used to trigger the (cancer) cell-specific (onco)lysis. These viral vectors are referred to herein as “oncolytic viruses”. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole.

Aspects of the invention are demonstrated by the following non-limiting examples.

### EXAMPLES

Within the Examples the sequence of the described CDP corresponds SEQ ID NO:5 described above.

### Examples Section A

### Production of an RBD-Targeting Conjugate Vaccine According to the Invention

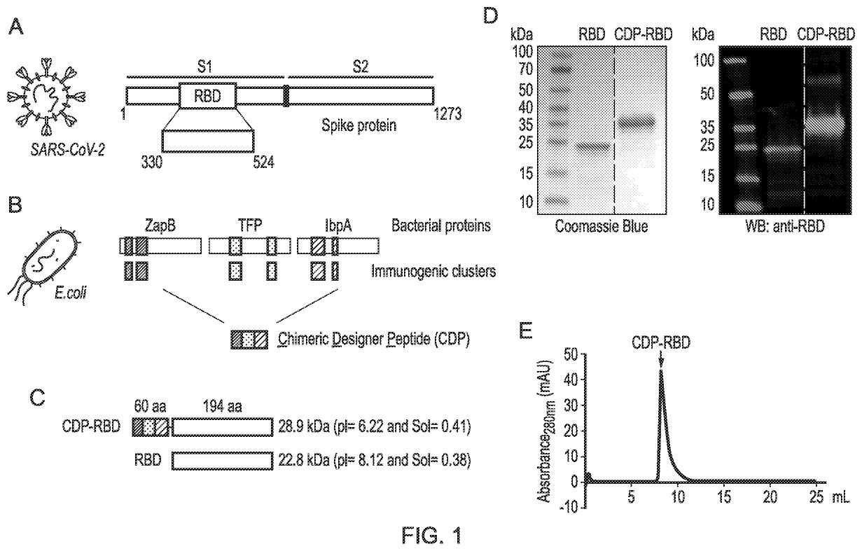

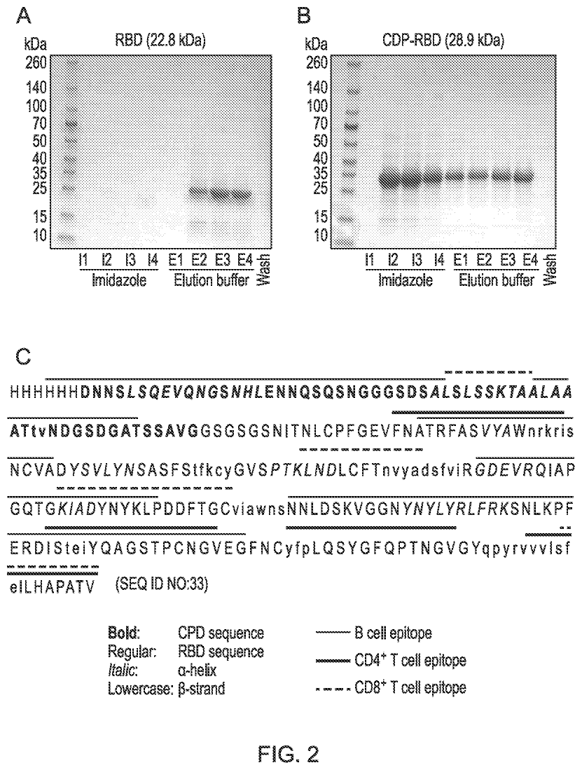

To achieve robust immune recognition of the RBD domain of SARS-CoV-2 (FIG. 1A), its sequence was conjugated to an engineered chimeric designer peptide (CDP) (FIG. 1B). CDP has selected clusters of amino acids with bulky hydrophilic or charged side chains, originating from three distinct bacterial (E. coli) proteins, the cell division protein ZapB (uniprot #P0AF36), the type I fimbrial protein (A chain) (TFP, uniprot #P04128) and the small heat shock protein IbpA (uniprot #P0C054). The resulting conjugate protein CDP-RBD, as well as RBD alone (FIG. 1C), were expressed in BL21 bacteria (E. coli strain) and subsequently purified as previously described. Notably, the addition of the CDP component to RBD improved the solubility of the final conjugate protein (FIG. 1C) and facilitated purification (FIG. 2A, 2B). Purified proteins were validated by SDS-PAGE and western blot analysis using a commercial anti-RBD antibody (FIG. 1D). Also, size exclusion chromatography confirmed the purity of CDP-RBD (FIG. 1E). Lastly, in silico analysis of the CDP-RBD protein sequence revealed the α-helix or β-strand domains, as well as the predicted B cell- and CD4+/CD8+ T cell epitopes (FIG. 2C), suggesting potent immune recognition.

### CDP-RBD Induces Faster and Stronger Antibody Responses Compared to RBD Alone

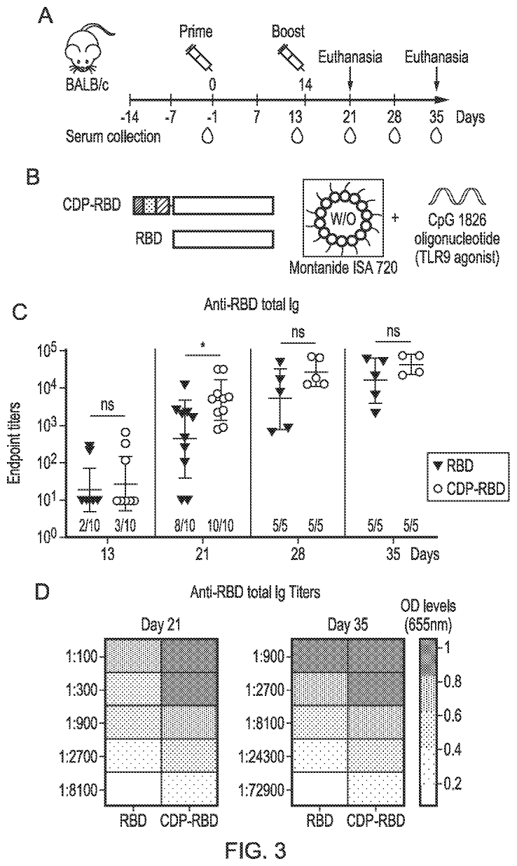

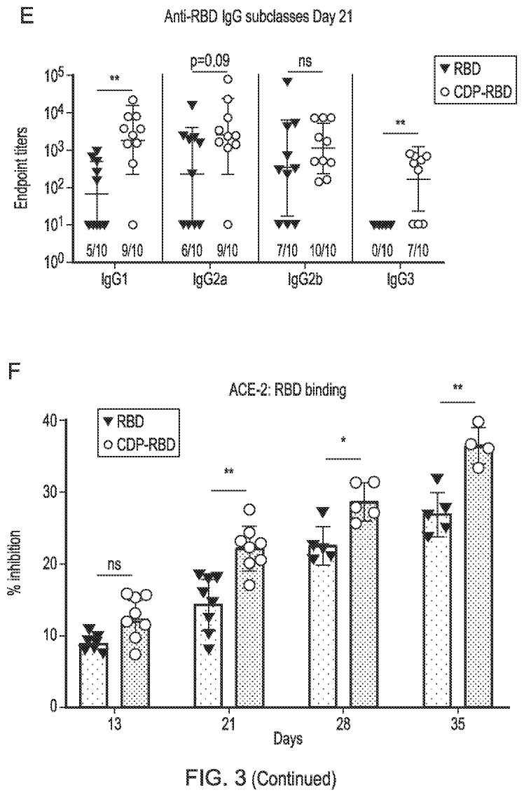

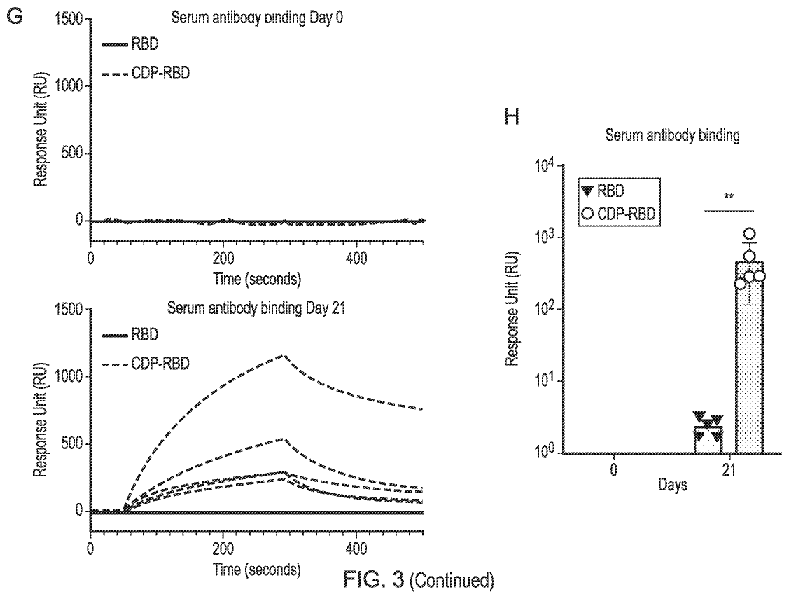

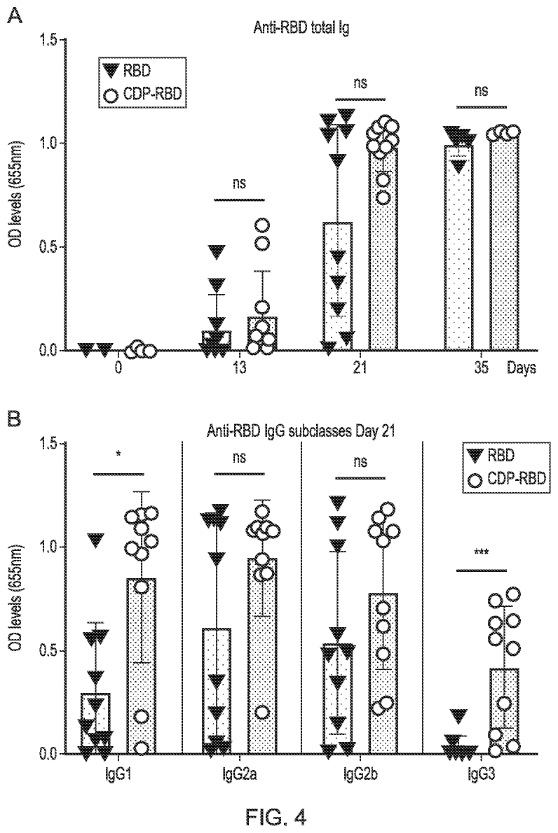

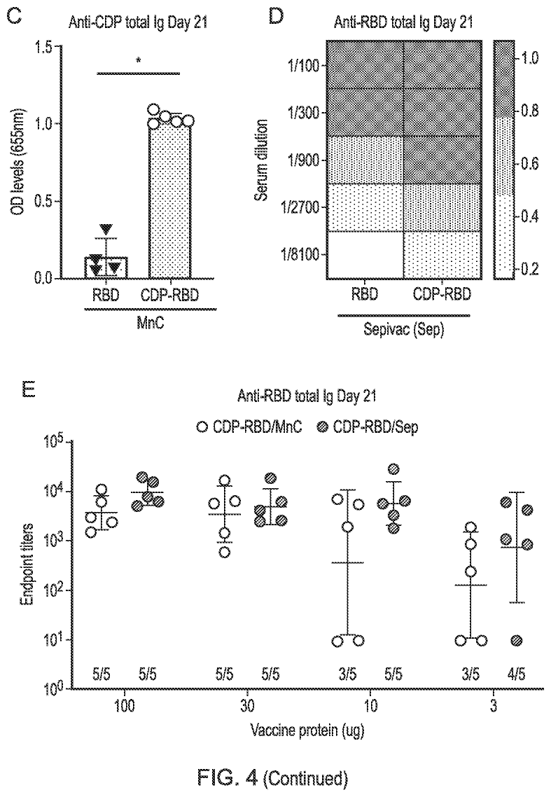

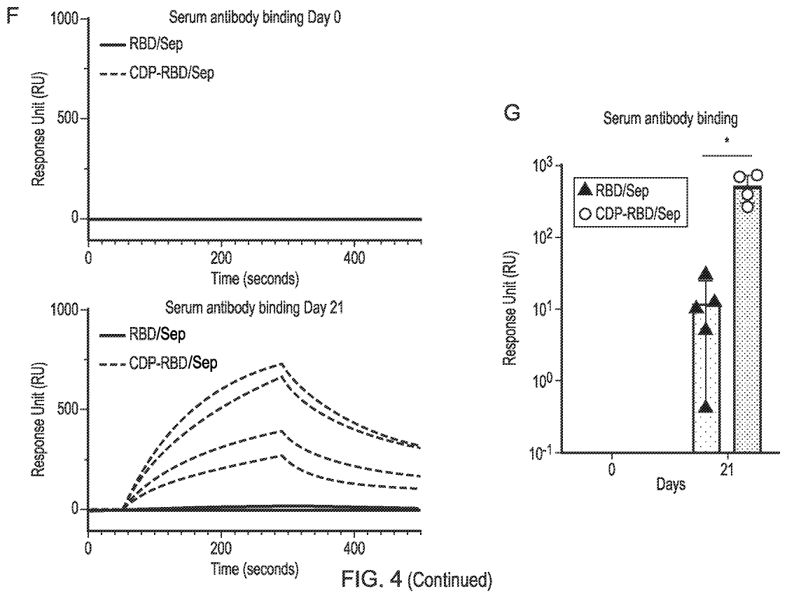

To assess the immunogenicity of CDP-RBD in vivo and compare it to its unconjugated RBD counterpart, BALB/c mice were immunized subcutaneously with 100 μg of each purified protein by a prime vaccination on day 0 and a booster vaccination on day 14 (FIG. 3A). Based on its previously reported safety and efficacy, Montanide ISA 720 (water-in-oil emulsion) supplemented with a CpG 1826 oligonucleotide (Toll-like receptor 9 agonist) was selected as vaccine adjuvant (referred to as MnC) (FIG. 3B). Murine sera were collected prior to immunization (day 0), as well as on days 13, 21, 28 and 35 (FIG. 3A) and were used for ELISA detection of anti-RBD antibodies. No antibodies against the RBD domain were present in the pre-immune sera (day 0). Seroconversion was detectable by day 13 in a couple of mice from each group (FIG. 3C and FIG. 4A) and by day 21 all mice immunized with CDP-RBD had developed a strong immunoglobulin (Ig) response against RBD. At this time point, only 50% of the RBD-immunized mice displayed a potent RBD-specific humoral response (FIG. 3C, 3D left and FIG. 4A), while no anti-RBD antibodies were detected in 2/10 mice receiving the unconjugated RBD vaccine (FIG. 3C). This result confirmed the moderate immunogenicity of the RBD. Importantly, although the difference in anti-RBD total Ig levels among the two vaccine groups seemed to disappear at later time points (FIG. 4A), evaluation of the antibody titers revealed that RBD-immunized mice were still lagging behind their CDP-RBD counterparts even three weeks after the booster injection (FIG. 2C, 2D right). These findings illustrate that using the iBoost platform for vaccination against RBD results in not only faster, but also stronger anti-RBD antibody responses relative to RBD alone. Next, the production of IgG subclasses was investigated. IgG1 and IgG2b are representative of T helper (Th)2-skewed immune responses, whereas IgG2a and IgG3 associate with induction of a Th1 response. By day 21, immunization of mice with CDP-RBD resulted in significantly higher levels of both anti-RBD IgG1 and IgG3 relative to vaccination with RBD (FIG. 3E and FIG. 4B). In addition, there was a trend towards a more robust and homogeneous IgG2a response in the CDP-RBD group (FIG. 3E and FIG. 4B). Interestingly, upon immunization with CDP-RBD, but not with unconjugated RBD, a higher number of mice were capable of mounting a complete IgG1, IgG2a, IgG2b and IgG3 response (FIG. 3E). For example, RBD-specific IgG3 was measured in 7/10 CDP-RBD-immunized mice, whereas none of the mice injected with RBD were found IgG3-positive (FIG. 3E and FIG. 4B). Hence, the CDP-RBD vaccine exhibits signs of an improved Th1-based immune response (indicated by higher IgG2a and IgG3 titers) and at the same time it ensures a more balanced Th1 and Th2 response (based on the presence of all IgG subtypes), as early as seven days after the second vaccination (day 21).

Given the vigorous immune recognition of RBD accomplished after conjugation to CDP, it was assumed that CDP-RBD recipient mice also develop anti-CDP antibodies. On day 21, CDP-specific antibodies were detected only in the CDP-RBD group (FIG. 4C). Taking into consideration the improved RBD-specific antibody response observed in mice immunized with the CDP-RBD conjugate vaccine, immune reaction to the bacterial CDP fusion partner did not seem to negatively influence the immunogenicity of the RBD antigen. Moreover, to exclude the possibility that the differences between the two vaccine groups are dependent on the adjuvant used, rather than the presence of CDP specifically, mice were immunized with RBD or CDP-RBD in combination with the alternative adjuvant Sepivac (Sep) in an identical experiment (FIG. 3A). In contrast to Montanide, which is a water-in-oil emulsion, Sepivac is an oil-in-water adjuvant that is currently being tested for several Influenza and COVID-19 vaccines. Again, CDP-RBD/Sep outperformed the RBD/Sep vaccine in terms of anti-RBD total Ig induction (FIG. 4D), in a similar manner as CDP-RBD/MnC, indicating that CDP is the key component responsible for boosting the humoral immune response. Importantly, CDP-RBD combined either with MnC or Sep was capable of inducing potent antibody responses against RBD even at lower protein amounts than 100 μg (FIG. 4E). In combination with Sepivac, CDP-RBD induces a potent immune response even at protein concentration of 10 μg (FIG. 4E). A surrogate neutralization assay revealed that sera derived from CDP-RBD-immunized mice displayed higher inhibition of RBD:ACE-2 binding compared to sera from RBD-immunized mice from day 21 onwards (FIG. 3F). This underscores the superior neutralization capacity obtained already one week after the second vaccination with the conjugate CDP-RBD vaccine. In line with this, surface plasmon resonance (SPR) analysis revealed that sera of mice vaccinated with CDP-RBD exhibited a significantly higher level of binding towards immobilized RBD than sera from RBD-vaccinated mice (FIG. 3G, H).

Systemic T Cell Immunity and Enhanced CD8⁺ T Cell Responses are Induced Upon Vaccination with CDP-RBD

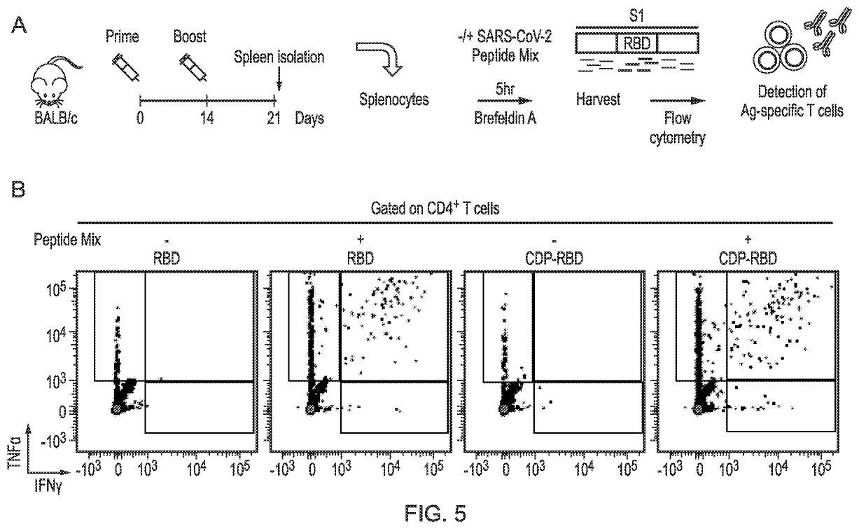

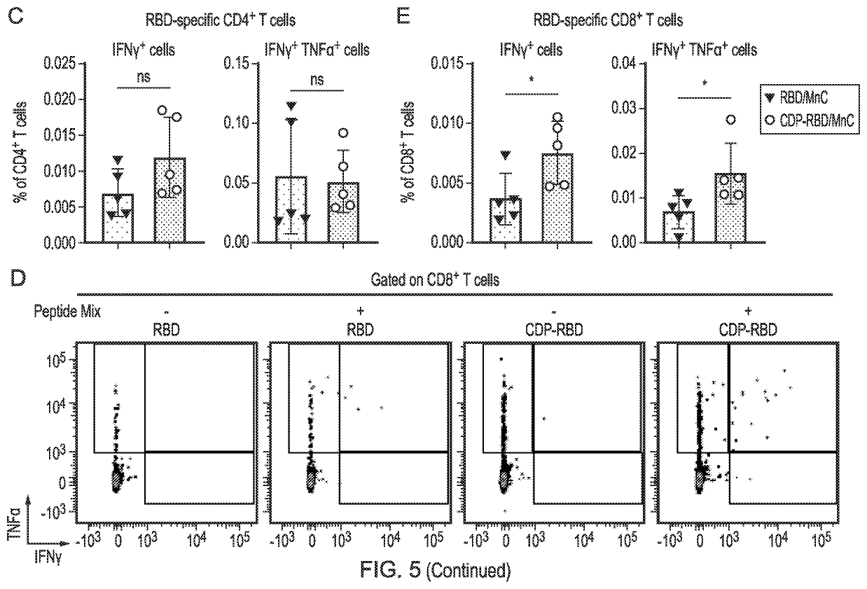

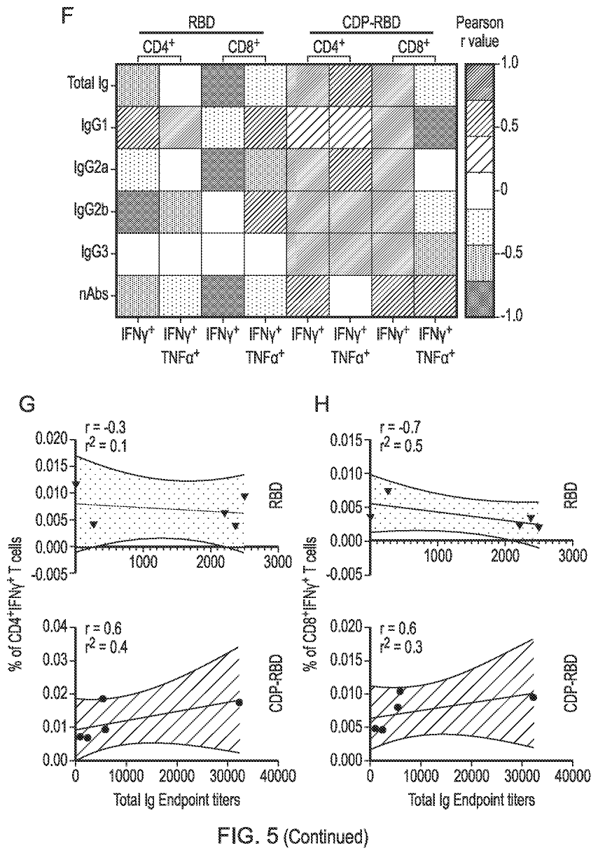

Besides antibodies, antigen-specific T cells are also crucial for the clearance of SARS-CoV-2 infection. Thus, next it was aimed to determine whether CDP-RBD induces stronger T cell responses in mice compared to unconjugated RBD. To assess this, the spleens of vaccinated mice were excised on day 21, since this was the point in time that the two vaccine groups started differing in their antibody responses and neutralization capacity. Splenocytes were restimulated ex vivo with a pool of 15 amino acid-long peptides covering the S1 domain (which includes RBD) of the spike protein (FIG. 5A). Non-stimulated splenocytes were taken along as a negative control. After stimulation for 5 hours in the presence of Brefeldin A, the frequency of antigen-specific CD4⁺ and CD8⁺ T cells was examined using flow cytometry (FIG. 6A), based on the induction of interferon gamma (IFNγ) and tumor necrosis factor alpha (TNFα) expression (FIG. 5B, 5D). More specifically, it was possible to identify a clear population of monofunctional (IFNγ⁺ or TNFα+), as well as multifunctional (IFNγ+TNFα+) CD4+ T cells (FIG. 5B, 5C and FIG. 6B), but without major discernible differences among the two vaccine groups. On the contrary, a significantly higher percentage of both monofunctional and multifunctional RBD-specific CD8+ T cells was detected in the spleens of CDP-RBD-immunized mice compared to their RBD-immunized counterparts (FIG. 5D, 5E and FIG. 6B), suggesting that conjugation to CDP has a positive impact on the systemic cytotoxic T cell (CTL) response. To further clarify the link between the humoral and cellular immune responses in these vaccine groups, the correlation between the anti-RBD antibodies and the RBD-specific T cells was tested (FIG. 5F). Interestingly, multiple components of the antibody response were found to be positively correlated with the T cell response in CDP-RBD-immunized mice (except for polyfunctional CD8+ T cells), while IgG1 was the predominant IgG subtype showing high correlation with T cells in the case of RBD recipients (FIG. 5F-3H). Taken together, these data suggest that the conjugate technology facilitates the induction of a coordinated and fine-tuned antibody and T cell response very quickly after the second vaccine dose.

### The CDP-RBD Vaccine Protects Against COVID-19 in Hamsters

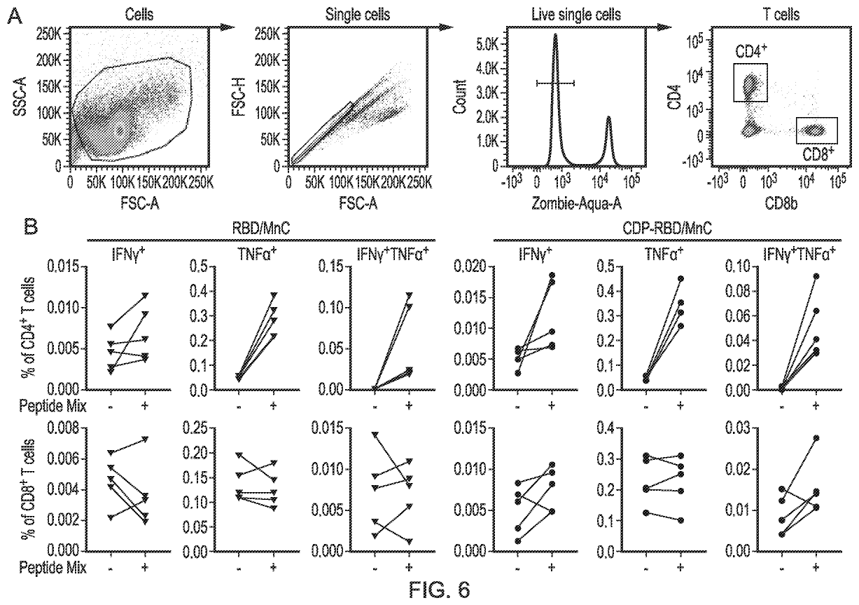

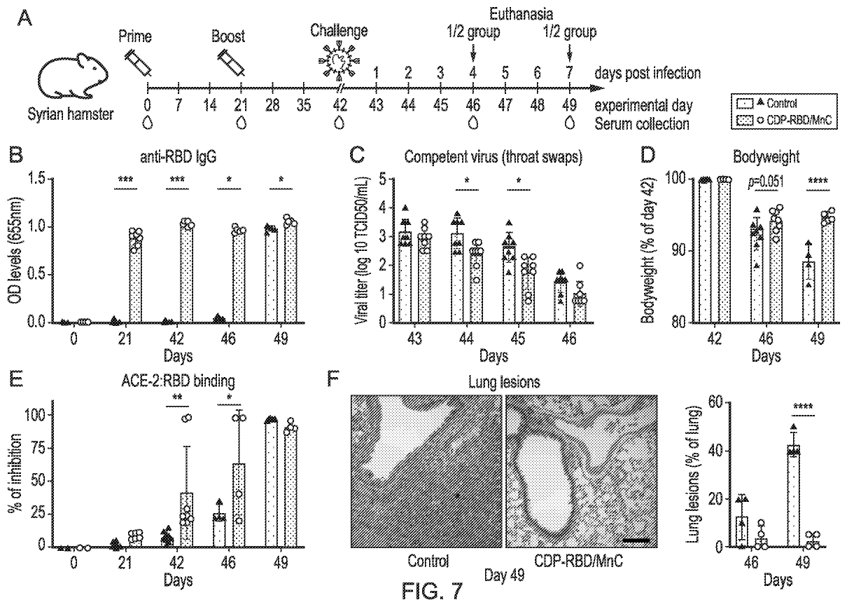

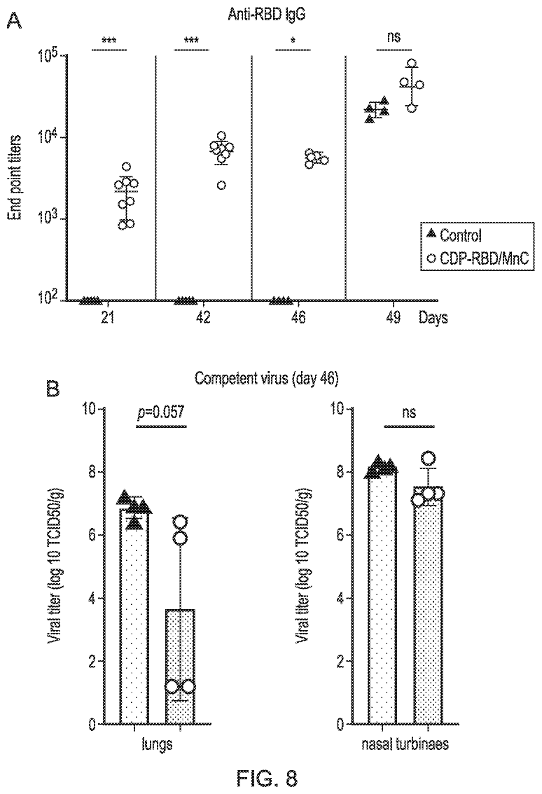

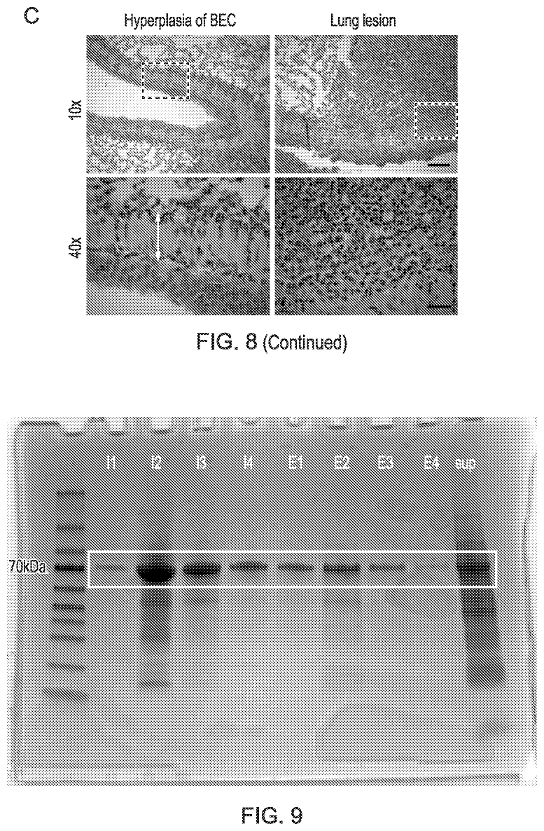

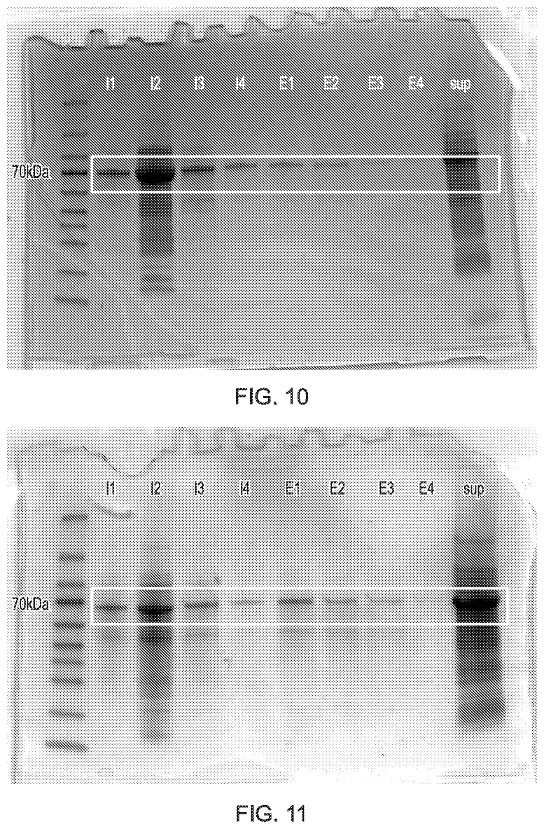

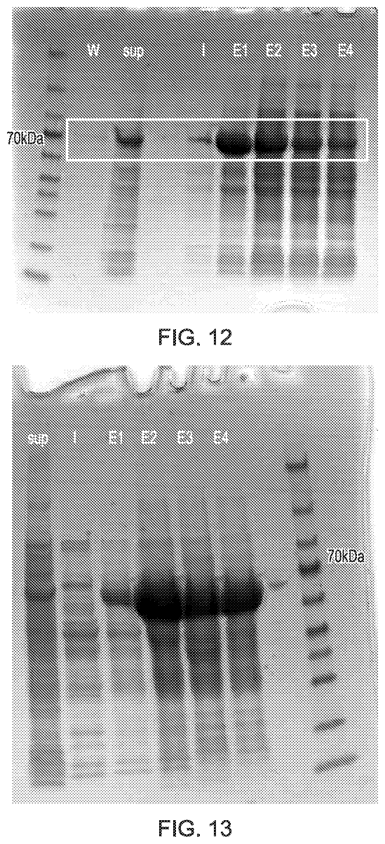

Further, it was investigated whether the CDP-RBD vaccine, besides inducing an RBD-specific antibody and T cell response, also offers protection against SARS-CoV-2 infection. Therefore, Syrian hamsters were immunized intramuscularly with CDP-RBD plus MnC or Tris-sucrose only (control) at day 0 and 21. Three weeks after the booster vaccination (experimental day 42), all hamsters were challenged intranasally with 10⁴ SARS-CoV-2 virus particles, which is equal to the median tissue culture infectious dose (TCID50), and serum samples were taken four and seven days post infection (dpi) (FIG. 7A). It was found that the CDP-RBD vaccine elicited high antibody titers against RBD after a single vaccine dose (day 21), while the booster vaccination increased the anti-RBD antibody titers further (FIG. 7B and FIG. 8A). As expected, control hamsters showed no anti-RBD antibodies after vaccination. However, seven days after challenge with SARS-CoV-2, control hamsters also displayed high anti-RBD antibodies, although these did not reach the antibody titers detected in the CDP-RBD vaccinated hamsters (FIG. 7B and FIG. 8A). In line with the higher antibody titers, CDP-RBD vaccinated hamsters showed a faster clearance of the virus from the throat. More precisely, at two and three dpi significantly lower amounts of replicating virus were found (FIG. 7C). Moreover, lower amounts of virus were found in the lungs, as well as in the nasal turbine, of CDP-RBD vaccinated hamsters at four dpi (FIG. 8B). After SARS-CoV-2 infection, both groups lost around 5% of their initial bodyweight in the first four dpi. Nevertheless, CDP-RBD vaccinated hamsters maintained their bodyweight from this time point on (day 46), whereas control vaccinated hamsters lost significantly more weight (FIG. 7D). To elucidate whether CDP-RBD-induced antibodies are characterized by neutralizing capabilities, a surrogate neutralization assay was performed (FIG. 7E). Antibodies of CDP-RBD-vaccinated animals hampered the binding of RBD to ACE-2 already at the day of viral challenge (day 42). Significantly improved neutralization compared to control continued at four dpi. One week after viral challenge (day 49), hamsters of both groups exhibited high inhibition of binding between RBD and ACE-2 (FIG. 7E). Strikingly, histopathological analyses performed on lung tissues showed typical COVID-19 characteristics like congestion, emphysema, haemorrhage, bronchioloalveolar hyperplasia and inflammation or edema in all control hamsters, but not in CDP-RBD vaccinated hamsters. Up to 40% of the lung tissue showed lesions in control hamsters seven dpi, whereas CDP-RBD vaccinated hamsters presented no evidence of SARS-CoV-2 pathology (FIG. 7F).

### DISCUSSION

Here, the idea of boosting the immunogenicity of a viral antigen by conjugating it to a bacterial chimeric sequence is presented. A critical component of the present invention is CDP (chimeric designer peptide), which is made up of bacterial protein sequences enriched in highly immunogenic amino acid clusters. CDP should also be beneficial for improving immune responses against critical, but only moderately immunogenic viral sequences, such as the RBD domain of the SARS-CoV-2 spike protein. Therefore, CDP was conjugated to the RBD sequence and this resulted in significantly improved immune recognition of the RBD antigen.

It was observed that immunization of mice with the conjugate CDP-RBD vaccine results in faster, stronger and more balanced IgG responses, as compared to vaccination with unconjugated RBD. Induction of an early immune response is considered of utmost importance during targeting highly infectious agents, like SARS-CoV-2, especially in a pandemic situation. Additionally, mature responses reduce the risk of COVID-19 mortality associated with a Th2/Th1 cytokine disparity. Here, an improved IgG response in the CDP-RBD group was observed. Notably, only CDP-RBD-immunized mice were able to produce IgG3, an IgG subclass that is associated with complement activation. Also, vaccination with CDP-RBD enhanced skewing towards a Th1 immune response (based on IgG2a and IgG3 levels), which is fundamental during viral infections and a prerequisite for the success of many vaccine candidates. Independently of the technology used, vaccine-induced generation of neutralizing antibodies is greatly dependent on CD4+ T cells with a Th1 phenotype. In relation to this, it was found that combination of CDP with RBD can significantly increase serum neutralization capacity as well as binding to RBD compared to RBD alone in mice from day 21 onwards. Also, using SPR analysis, we found that CDP-RBD-derived serum antibodies bind much stronger to immobilized RBD, which is indicative of higher avidity and consistent with the improved inhibition of ACE-2:RBD binding.

Besides humoral immunity, the induction of cellular immune responses is essential for protection against viruses, especially since it has been shown that antibodies wane soon after infection or vaccination. Along with the numbers, the functionality of SARS-CoV-2-specific T cells determines the timing and the efficiency of virus clearance from the body and it is inversely correlated with disease severity. Furthermore, certain SARS-CoV-2 variants of concern are unable to escape recognition by S-specific memory T cells, as it often happens with antibodies. For this reason, special attention is given to the generation of SARS-CoV-2-specific T cells upon vaccination. An established method for the ex vivo assessment of antigen-reactive T cells either in mice or humans includes the quantification of cells expressing the Th1 cytokines IFNγ and TNFα after re-stimulation with a pool of overlapping peptides corresponding to the target antigen. In this study, CDP-RBD-immunized mice exhibited both CD4+ and CD8+RBD-specific T cells in their spleen, indicative of successful induction of a systemic cellular response one week after the booster vaccination. Despite the differences in the total Ig response, a similar frequency of RBD-specific CD4+ T cells among the two groups was observed. Nevertheless, compared to immunization with unconjugated RBD, CDP-RBD increased the frequency of reactive monofunctional and multifunctional CD8+ T cells, which are supported by Th1 cells and exert a pivotal role in killing infected cells upon encounter of their cognate antigen. This suggests that the use of the present invention can improve the underlying cytotoxic T cell responses and thus, increase protection in vaccinated individuals, since CD8+ T cells specific for certain coronavirus epitopes correlate with the development of mild COVID-19. It was also noticed that the proportion of splenic RBD-specific CD8+ T cells was smaller than the one of CD4+ T cells. This is in agreement with previous reports showing that the memory response in the context of SARS-CoV-2 is skewed more toward the CD4⁺ rather than the CD8+ T cell compartment. Finally, CDP-RBD-immunized mice displayed better correlation of the antibody response to the cellular response compared to their RBD counterparts. This suggests that the presentation of a viral antigen in the context of a conjugate vaccine with a selected foreign partner has the potential to mobilize both arms of the adaptive immune response in a more robust and well-orchestrated manner than that of the viral antigen alone.

Apart from the improved immune response in mice, the data also demonstrate that CDP-RBD protects against severe COVID-19 in a hamster model. CDP-RBD-vaccinated hamsters showed strong antibody responses and no lung lesions, as well as decreased weight loss, compared to controls after SARS-CoV-2 challenge. Protection was achieved despite the minor ˜0.5 log 10 TCID50 reduction of competent virus in the throat (per mL) and nasal turbines (per gram tissue). However, CDP-RBD vaccinated animals showed ˜3 log 10 TCID50/g reduction of competent virus in the lungs four dpi, suggesting that protection of lung lesions is strongly dependent on the viral load in the lungs rather than in the upper respiratory tract. This observation is in line with SARS-CoV-2 infection in humans, where replication of the virus in the lower airways is the main cause for increased mortality. Furthermore, a decrease of ˜3 log 10 TCID50/g in the lungs is comparable to the reduction of specific variants of concern after vaccination with the approved ChAdOx1 nCoV-19 (AZD1222) vaccine. In line with this observation, the two animals which showed no signs of lung lesions at experimental day 46 are the same animals that showed a strong reduction of competent virus in the lungs (˜5 log 10 TCID50/g) and high levels of neutralizing antibodies (˜100% inhibition of ACE-2:RBD binding). CDP-RBD-stimulated neutralizing antibody levels were comparable with those upon natural infection one week after the challenge. Although natural infection resulted in high neutralizing antibody levels after seven days, moderate neutralization was already achieved three weeks after the second vaccination/the day of viral challenge in CDP-RBD-immunized hamsters. Furthermore, significantly improved neutralization capabilities were observed four dpi, underlining the importance of an early manifestation of neutralizing antibodies for viral clearance and patient survival.

In general, protein subunit vaccines are a well-established technology with proven efficacy and safety profiles for many years, inter alia, pneumococcal polysaccharide or MenACWY vaccines. Moreover, protein vaccines are relatively stable at a refrigerator-friendly 2-8° C. range, and are therefore easy to distribute and more economic to implement, especially in countries with limited resources. The present invention described herein does not only induce faster and stronger B- and T cell immune responses, it also allows specific targeting of critical epitopes within key viral proteins (such as the receptor-binding domain of the spike protein). Furthermore, the present invention offers the possibility to display domains, regardless of their original immunogenicity, of different viral variants (e.g. the delta and omicron SARS-CoV-2 variants) or even conserved sequences (e.g. among SARS-CoV-1, -2 and MERS-CoV). This way, the present invention can facilitate the design and further development of multi-epitope vaccines that induce a more targeted immune response and thereby might offer protection against new circulating variants. In sum, the present invention represents a promising approach for future vaccination strategies against different viruses and pathogenic microorganisms.

### Material & Methods

### Vaccine Design

The genome of Escherichia coli (E. coli) (strain K12) was examined and three proteins highly enriched in clusters of amino acids with hydrophilic or charged side chains were identified: type-1 fimbrial protein TFP, cell division protein ZapB and the small heat shock protein IbpA. These immunogenic clusters were fused in order to form one single peptide, named chimeric designer peptide (CDP), with a total length of 60 amino acids (5.8 kDa). For the RBD, the sequence encoding amino acids 330 until 524 (in total 194 amino acids) of the reported SARS-CoV-2 genome (GenBank: MN908947.3) was used. Additionally, a (GS)₃-linker (GSGSGS) between the CDP and RBD domain, as well as a terminal Hiss-tag (HHHHHH), were added to facilitate proper antigen display and vaccine purification, respectively.

### Vaccine Protein Production

RBD and CDP-RBD proteins were expressed and purified as previously described (Huijbers et al., 2018). In brief, the sequences encoding for RBD or CDP-RBD (codon optimized for expression in E. coli) were synthesized by Genscript (USA) and inserted between the Nde1 and Xho1 restriction sites of a pET21a (+) expression vector. For protein expression, 10 ng plasmid DNA was transfected by the heat shock method into competent BL21 (DE3) E. coli bacteria (Sigma-Aldrich, #69450). A single clone was cultured overnight in LB-medium supplemented with ampicillin (100 μg/ml) in a non-humified shaker (Laboshake) at 200 rpm and 37° C. 1 mM Isopropyl β-d-1-thiogalactopyranoside (IPTG) (Serva, #26600.04) was added to a 1:2.6-diluted overnight culture to induce protein expression. After 4 h, bacteria were harvested and bacterial pellets (equivalent to 50 ml culture volume) were dissolved in 5 ml sonication buffer, containing 0.5 M EDTA (Sigma-Aldrich, #E5134), 1% N-lauroylsarcosine (Sigma-Aldrich, #L5125), 1% Phenylmethanesulfonyl fluoride solution (PMSF) (Sigma-Aldrich, #93482) and 6 M Urea (Acros Organics, #10665572) in PBS (VWR, #L0500-500). Proteins were released from the bacteria by high frequency vibration (sonication) for 15 cycles of 20 seconds “on” and 30 seconds “off” on ice (Soniprep 150 MSE, amplitude 22-24 microns). For protein purification, the supernatants (after centrifugation at 4500 rpm for 20 min) of sonicated samples were mixed with 200 pI 50% Ni-NTA agarose slurry (Qiagen, #30210) and incubated on a roller bench overnight at 4° C. The next day, Ni-NTA agarose beads were washed five times with wash buffer, containing 1 M NaCl (VWR, #27788.366) and 0.05% Tween-20 (Sigma-Aldrich, #P7949) in PBS. Subsequently, beads were transferred to a column, a syringe with a glass filter (Sartorius, #13400-100—K). Proteins were eluted in two steps: first four fractions of 100 pI each were eluted in 1 M Tris-CI (pH 8.0), 100 mM NaCl, 200 mM imidazole (JT Baker, #1747.0100), 1 mM PMSF and secondly four fractions of 100 μl each in 10 mM Tris-CI (pH 4.5), 100 mM NaH₂PO₄ (Merck, #1.06346.1000), 8 M urea. CDP-RBD was eluted in all eight fractions, whereas RBD was eluted only in the last four fractions containing 8 M urea. Fractions of purified proteins were pooled and stepwise dialyzed (4 M, 3 M, 2.5 M, 2 M urea) against 2 M urea. Final protein concentration was determined by BCA assay (Thermo Scientific, #23235). SDS-PAGE, Coomassie blue staining (Coomassie® Brilliant Blue G 250, Serva, #17524.02), western blot analysis, and size exclusion chromatography were performed to confirm purity and identity of the proteins.

### Western Blot

Identity of the proteins was confirmed by western blot. Approximately 20 μg of purified protein was loaded on Mini-PROTEAN® TGX™ precast protein gels (Biorad, #4561094, 4561096) and gel electrophoresis was performed. Subsequently, proteins were transferred to an immobilon PVDF membrane (Millipore, #IPFL00010). Membranes were blocked with PBS containing 0.05% Tween-20 (PBS-T) and 5% BSA fraction V (Roche, #10735086001) for 1 h at room temperature. Rabbit anti-RBD antibody (R&D systems, #MAB10540, 1/250 dilution) was used as primary antibody overnight at 4° C. The next day, 5 washes with 0.05% PBS-T were performed before adding the secondary antibody, goat anti-mouse IRDye 8000W (LI-COR Biosciences, #926-32210, 1/10000 dilution), for 30 min to the membranes. After five washes with PBS-T and one wash with PBS, proteins were visualized with an Odyssey Infrared Imaging System (Model 9120, LI-COR Biosciences). Prestained spectra multicolor broad range protein ladder (Thermo Fisher, 26634) was used as size reference.

### Analytical Gel Filtration Assay

Analytical gel filtration was conducted using the AKTA Pure system equipped with a Superdex 75 10/300 GL column (Cytiva) in 2 M urea in phosphate-buffered saline (PBS, pH 7.4) at a flow rate of 0.5 ml/min. Each injection contained 100 pI sample with a concentration of 2 g l⁻¹. The absorbance was monitored at 280 nm.

Prediction of B, CD4⁺ and CD8⁺ T Cell Epitopes

Linear B cell epitopes in the CDP-RBD sequence were predicted by BepiPred applying an epitope threshold of 0.55. For the prediction of potential CD4+ T cell epitopes we exploited the NetMHCllpan 4.0 server. Specifically, the affinity of all the overlapping 15 amino-acid long peptides included in the CDP-RBD sequence was tested for binding to the murine H-2 alleles (H-2-lau, H-2-led, H-2-lek), as well as to several common human HLA-DP, HLA-DQ and HLA-DR alleles. Similarly, we identified predicted CD8+ T cells epitopes of 8 amino-acid length in CDP-RBD using the NetMHCpan 4.1 server. The binding to the murine H-2 alleles (H-2-Db, H-2-Dd, H-2-Dq, H-2-Kb, H-2-Kd, H-2-Kk, H-2-Kq, H-2-Ld, H-2-Lq) and different human HLA class I alleles was examined.

### Mouse Vaccinations

All mouse experiments were approved by the Dutch national ethics board Centrale Commissie Dierproeven (CCD, registration number AVD11400202010545) and were performed in agreement with Dutch guidelines and law on animal experimentation. Female 8 weeks-old BALB/c OlaHsd mice (Envigo) were used for two independent immunization studies (n=5 per group each). One study was terminated at day 21 and the other study at day 35. After acclimatization for 2 weeks, mice received a prime vaccination on day 0 and a booster vaccination on day 14 (FIG. 3A). Each mouse was injected subcutaneously in the left groin with 100 μg of purified CDP-RBD or RBD, respectively. Proteins were mixed 40:50:10 with Montanide ISA 720 (Seppic, #36059VFL2R3) and 50 μg CpG oligo 1826 (Eurogentec, #1826), abbreviated as MnC. For the concentration study, every mouse was injected subcutaneously in the left groin with either 100, 30, 10 or 3 μg of purified RBD or CDP-RBD in combination with Montanide ISA 720 and CpG (40:50:10, MnC) or Sepivac SWE (Seppic, #1849101, 50:50), respectively. Blood samples were collected from the tail vein prior to immunization (day 0) and on days 13, 21, 28 and 35. One mouse from the CDP-RBD MnC group was sacrificed between day 28 and day 35 due to an open wound at the vaccine injection site. On day 35, all the remaining mice were sacrificed.

### Detection of Anti-RBD Antibodies in Mouse Serum by ELISA

After overnight coagulation at 4° C., blood samples were centrifuged twice at 7000 rpm (10 min, 4° C.). Sera were collected and stored at −20° C. until further use. Recombinant RBD (GenScript, #Z03483) was used to coat flat-bottom 96-well plates (Thermo Fisher Scientific, #442404) at a final concentration of 2 μg/mL (total Ig plates) or 0.5 μg/mL (IgG1, IgG2a, IgG2b, and IgG3 plates) in PBS for 1 h at 37° C. Plates were washed once with PBS containing 0.1% Tween-20 (PBS-T) and blocked with 1% non-fat dry milk (Santa Cruz, #SC-2325) in PBS-T for 1 h at 37° C. After one wash with PBS-T, serial dilutions (1/100 to 1/72900 in blocking solution) of mouse sera were added and incubated for 45 min. at 37° C. A monoclonal antibody against the RBD of SARS-CoV-2 (R&D Systems, 1/500 dilution) was used as a positive control. Plates were washed four times with PBS-T. Biotinylated goat anti-mouse total Ig (Dako, #E0433), IgG1 (Southern Biotech, #1070-08), IgG2a (Southern Biotech, #1080-08), IgG2b (Southern Biotech, #1090-08) and IgG3 (Southern Biotech, #1100-08) antibodies diluted in PBS-T (1/2000) were incubated for 45 min. at 37° C. After four washes with PBS-T, plates were incubated with Streptavidin-HRP (Dako, #P0397) diluted in PBS-T (1/2000) for 30 min. at 37° C. Plates were washed four times with PBS-T and developed with TMB (Sigma, #T0440) for 10 min. The absorbance was measured on a microplate reader (BioTek Synergy HTX) at 655 nm. Actual values were obtained by subtracting blank (wells that were treated like sample wells except adding blocking solution instead of mouse sera) OD values from the actual OD values.

### Neutralization Assay

To measure the neutralizing capacity of induced anti-RBD antibodies, the SARS-CoV-2 surrogate virus neutralization assay from Genscript (USA, #L00847) was performed according to the manufacturer's instructions.

### Spr Biosensor Assay

Surface Plasmon Resonance (SPR) biosensor assays have been carried out using Biacore T200 (GE Healthcare) with CM5 sensor chips (Cytiva). RBD (R&D systems) at a concentration −6 g/ml in 10 mM acetate buffer pH 4.5 was immobilized at the density of ˜2 kRU using the amine-coupling kit (Cytiva) according to the manufacture protocol at the flowrate 5 I/min. For binding analysis, serum samples were diluted 1:100 in PBS buffer pH 7.4 supplemented with 0.05% Tween-20 and injected over the sensor chip surface at 30 ul/min flowrate, 25° C. for 240 sec. Dissociation of formed complexes was followed for 180 sec after an end of an injection. After each cycle the chip surface was regenerated by 30 sec injections of 10 mM Gly, pH 2 and 0.5 M urea.

### Detection of Anti-CDP Antibodies in Mouse Serum by ELISA

CDP was fused to truncated (first 58 C-terminal amino acids) bacterial thioredoxin (uniprot #P0AA25) (TRXtr-CDP). TRXtr-CDP was produced in BL21 (DE3) E. coli bacteria (Novagen) as described above and used to coat a flat-bottom 96-well plates (Thermo Fisher Scientific) at a final concentration of 2 μg/mL in PBS for 1 h at 37° C. Plates were washed and blocked as described above. Mouse sera were diluted in 100% BL21 extract, serial dilutions were added and incubated for 45 min. at 37° C. For detection of anti-CDP antibodies, a biotinylated goat anti-mouse total Ig (Dako) antibody, diluted in PBS-T (1/2000), was incubated for 45 min. at 37° C. Plates were washed four times with PBS-T and they were incubated with Streptavidin-HRP (Dako) diluted in PBS-T (1/2000) for 30 min. at 37° C. Plates were further washed, developed and analyzed as described above.

### Splenocyte Isolation and Restimulation

Spleens from mice immunized with RBD/MnC or CDP-RBD/MnC were isolated on day 35, cut into small pieces and mechanically dissociated. Splenocytes were passed through a 70 μm cell strainer (Corning, #431751) and spun down for 5 min at 1500 rpm (brake 5, room temperature) in a Rotina 420 R (Hettich) centrifuge. Red blood cells were lysed upon incubation with ammonium-chloride-potassium lysis buffer (150 mM NH₄Cl, 10 mM KHCO₃, 100 mM EDTA, pH=7.4) for 3 min. at room temperature. Splenocytes were washed once and then resuspended in RPMI-1640 (Gibco, #L0495-500) medium supplemented with 10% FCS (Biowest, #S0750), 1% Pen/Strep (Gibco, #15140-122), 1% L-glutamine (Brunschwig Chemie BV, #HN08.2) and 50 μM 2-mercaptoethanol (Gibco, #31350-010). Isolated splenocytes were seeded in U-bottom 96-well plates (Greiner Bio-One, #650180) (2*10⁶ cells/well) and were restimulated ex vivo for 5 h with a SARS-CoV-2 peptide mix (Miltenyi Biotec, #130-127-041) in the presence of Brefeldin A (BioLegend, #420604). Peptides of 15 amino acid length with 11 amino acid overlap were used, covering the S1 domain (which contains RBD) of the spike glycoprotein. Unstimulated splenocytes treated with Brefeldin A were taken along as a negative control.

Identification of RBD-Specific T Cells with Flow Cytometry

After restimulation, cells were transferred to a V-bottom 96-well plate (Greiner Bio-One, #651180) and washed once with PBS. Cells were resuspended in TruStain Fc blocking solution (BioLegend, #101320) for 10 min. at room temperature. Afterwards, cells were incubated with anti-mouse CD4-AF700 (BioLegend, #100429, 1/200 dilution), anti-mouse CD8b-AF488 (BioLegend, #126627, 1/200 dilution) and Zombie Aqua fixable viability dye (BioLegend, #423101, 1/200 dilution) diluted in PBS for 20 min. on ice. Cells were washed once with PBS and fixed with 4% paraformaldehyde (PFA; Electron Microscopy Sciences, #15710) for 15 min. on ice. After fixation, cells were washed once with PBS and permeabilized using the intracellular staining permeabilization wash buffer (BioLegend, #421002) following manufacturer's instructions. Cell suspensions were then incubated with anti-mouse IFNγ-APC (BioLegend, #505305, 1/200 dilution) and TNFα-PE (BioLegend, #506305, 1/200 dilution) diluted in intracellular staining permeabilization wash buffer for 30 min. at room temperature. Cells were washed twice with the permeabilization wash buffer, resuspended in 100 pI PBS and transferred to FACS tubes. Fluorescence intensities and the percentage of IFNγ- and TNFα-expressing cells were measured using an LSRII (BD Biosciences) flow cytometer. Data analysis was performed with the FlowJo V10 software (Tree Star).

### Hamsters

Male Syrian hamsters (Mesocricetus auratus) were purchased at Janvier (France). The animals were 9 weeks old and weighed 98-114 g at the start of the experiment. Hamsters were housed in type 2 cages with a maximum of two animals per cage under DM(BSL)-II conditions during the acclimatization and vaccination phase. Hamsters were transferred to standard elongated type 2 group cages with two animals per cage under BSL-Ill conditions (isolators) on the day of virus inoculation using sawdust as bedding with cage enrichment. For all invasive animal procedures, intranasal and intramuscular administration, blood sampling, throat swab collection and euthanasia, the animals were sedated with isoflurane (3-4%/02). Hamsters were vaccinated according to the schedule via the intramuscular route (i.m) (FIG. 7A). Blood was taken via orbital bleeding. Blood samples for serum were immediately transferred to appropriate tubes containing a clot activator. Serum was collected, heat treated, aliquoted and stored frozen.

### Hamster Vaccination

On day 0 and 21 animals were vaccinated with either Tris-sucrose as control or 100 μg CDP-RBD (mixed with Montanide ISA 720 and 67 μg CpG oligo 1826, MnC) in a total volume of 100 pI intramuscularly (i.m). Hamsters were injected with a syringe fitted with a 29G (0.33×12.7 mm) needle into both hind legs. In short, the hind limb was extended and inoculum injected with a short fluid movement into the outer thigh (biceps femoris), avoiding the caudal muscles to prevent risk of damage to the sciatic nerve. Animals were placed back in the cage and monitored during recovery.

### SARS-CoV-2 Inoculation

On day 42, all hamsters were challenged with 10⁴ median tissue culture infectious dose (TCID50) SARS-CoV-2 virus particles (BetaCoV/Munich/BavPat1/2020, European Virus Archive Global) intranasal (i.n.) using a dose volume of 100 pI inoculum. On day 4 post challenge half of the animals per group were euthanized by exsanguination under isoflurane anesthesia and necropsy was performed. On day 7 post challenge, the remaining half of the animals per group were euthanized by exsanguination under isoflurane anesthesia and necropsy was performed.

### Detection of Anti-RBD Antibodies in Hamster Serum by ELISA

ELISA was performed as described above. A biotinylated goat anti-hamster IgG antibody (Southern Biotech, #6060-08, 1/2000 dilution) was used to detect hamster antibodies.

### Sampling Post Inoculation

Samples from the respiratory tract were collected daily during the challenge phase of the study. In short, throat swabs (FLOQSwabs, COPAN Diagnostic Inc., Italy) were used to sample the pharynx by rubbing the swabs against the back of the animal's throat saturating the swab with saliva. Subsequently, the swab was placed in a tube containing 1.5 mL virus transport medium (Eagles minimal essential medium containing Hepes buffer, Na bicarbonate solution, L-Glutamin, Penicillin, Streptomycin, BSA fraction V and Amphothericine B), aliquoted in three aliquots and stored.

### Detection of Replication Competent Virus

Quadruplicate 10-fold serial dilutions were used to determine the virus titers in confluent layers of Vero E6 cells. To this end, serial dilutions of the samples (throat swabs and tissue homogenates) were made and incubated on Vero E6 monolayers for 1 h at 37° C. Vero E6 monolayers were washed and incubated for 5 or 6 days at 37° C. Plates were scored based on the cytopathic effect (CPE) by scoring using the vitality marker WST8. Therefore, WST-8 stock solution was prepared and added to the plates. Per well, 20 μL of this solution (containing 4 μL of the ready-to-use WST-8 solution from the kit and 16 μL infection medium, 1:5 dilution) was added and incubated 3-5 h at room temperature. Subsequently, plates were measured for absorbance at 450 nm (OD₄₅₀) using a micro plate reader and visual results of the positive control CPE were used to set the limits of the WST-8 staining (OD value associated with cpe). Viral titers (log 10 TCID50/ml or/g) were calculated using the method of Spearman-Karber.

### Lung Histopathology

Tissue samples (trachea, left lung and left nasal turbinates) were collected, inflated and/or stored in 10% formalin. After fixation, tissues from left lung and left nasal turbinate, gastrointestinal tract were embedded in paraffin. Tissue sections were stained with hematoxylin/eosin for histological examination. Histopathological assessment included aspects like congestion, emphysema, presence of foreign body, haemorrhage, bronchioloalveolar hyperplasia and inflammation and oedema.

### Statistical Analysis

The significance of the difference between experimental groups was evaluated by an unpaired, Mann-Whitney test, unpaired Student's t test or a two-way ANOVA followed by Sidka's multiple-comparison by Prism (GraphPad) software. A value of P<0.05 was considered significant.

By Fusing an iBoost Designer Partners (IDP) to Vimentin the Overall pI of the Fusion Protein is Lowered

Proteins with a His-tag (6×Hisitidne) can be eluted from Nickel-agarose with an excess amount of imidazole because the imidazole competes with the histidines for Nickel-agarose binding. If the protein is bound by charge to the Nickel-agarose it cannot be eluted from the Nickel-agarose by imidazole but only by a change to low pH, to give the protein a positive charge so that it will be eluted from the Ni-agarose.

By fusing IDP1 (SEQ ID NO:6), IDP2 (SEQ ID NO:7) or IDP3 (SEQ ID NO:8) to mouse vimentin the overall isoelectric point (pI) of the protein is lowered.

The isoelectric point of different constructs described herein is as follows: IDP1-mVim has an overall pI of 4.65; IDP2-mVim has an overall pI of 4.71; IDP3-mVim has an overall pI of 4.81; CDP (SEQ ID NO:5)-Vim has an overall pI of 5.09; and TRXtr (SEQ ID NO:10)-Vim has an overall pI of 5.48. For full length sequences of these constructs see Seq ID NO:s 34 to 38. For these constructs mouse vimentin was used (UniProtKB—P20152 (VIME-MOUSE)).

Lowering the overall pI of the fusion protein enhances the solubility of the vimentin protein meaning that elution is possible with imidazole buffer. For the fusion proteins CDP-Vim and TRXtr-Vim it is not possible to elute the protein with imidazole buffer, but only with buffer E.

### Examples Section B

Purification of mammalian peptide, Apelin, conjugated to immune stimulating peptides of SEQ ID NOs: 5 (CDP), 6 (CDP2.1), 7 (CDP2.2) and 10 (TRXtr). Mammalian peptide, apelin derived from mouse, was conjugated to immune stimulating peptides disclosed herein, namely SEQ ID NOs: (“CDP”), 6 (“CDP2.1”), 7 (“CDP2.2”) and 10 (“TRXtr” or “TRXtrunc”).

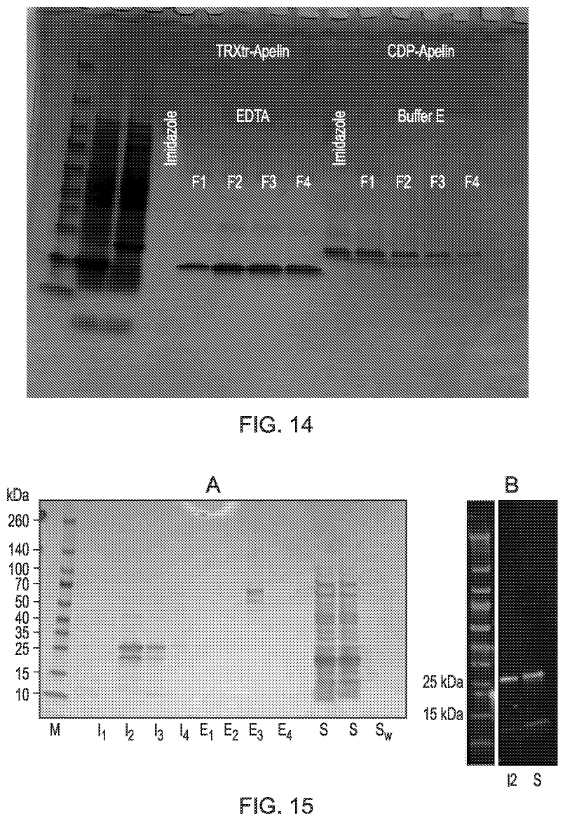

Regarding protein purification, TRXtr-Apelin was not eluted with 4 fractions of 200 mM imidazole (pooled on gel), but only eluted in buffer containing 100 mM EDTA (F1-F4). EDTA buffer may strip Nickel ions from agarose beads. When apelin is conjugated to CDP it was observed that protein was eluted in 4 fractions of 200 mM imidazole (pooled on gel) and in the buffer E fractions (FIG. 14). It was observed that expression of unconjugated Apelin could never be induced in E. coli and that protein could also not be purified.

Conjugation of mouse Apelin to CDP2.2 (SEQ ID NO:7) drastically decreased the isoelectric point (pI) of the fusion protein construct and made elution of the protein with 200 mM imidazole possible (11-14) (FIG. 15). The protein mainly eluted in the imidazole fractions, meaning that imidazole competed with histidine binding and that elution is not dependent on low pH (buffer E) and thus changing of charge of the protein.

The table below summarises various properties of mouse Apelin, and mouse Apelin fused to the C-terminus of each of TRXtr (SEQ ID NO:10), CDP (SEQ ID NO:5), CDP2.1 (SEQ ID NO: 6), CDP2.2 (SEQ ID NO:7) and CDP 2.3 (SEQ ID NO:8):

TABLE 1

| | MW | | pl | Solubility |
|---|---|---|---|---|
| Apelin | 9.6 | kDa | 11.65 | 0.615 |
| TRXtr-Apelin | 16.2 | kDa | 10.80 | 0.679 |
| CDP-Apelin | 16.0 | kDa | 9.88 | 0.561 |
| CDP2.1-Apelin | 17.9 | kDa | 5.53 | 0.599 |
| CDP2.2-Apelin | 16.7 | kDa | 4.73 | 0.648 |
| CDP2.3-Apelin | 14.4 | kDa | 5.73 | 0.470 |

### Purification of Mammalian Peptide, Timp1, Conjugated to Immune Stimulating Peptides, CDP (SEQ ID NO:5), CDP2.1 (SEQ ID NO:6), CDP2.2 (SEQ ID NO:7), CDP2.3 (SEQ ID NO:8) and TRXtr (SEQ ID NO:10).

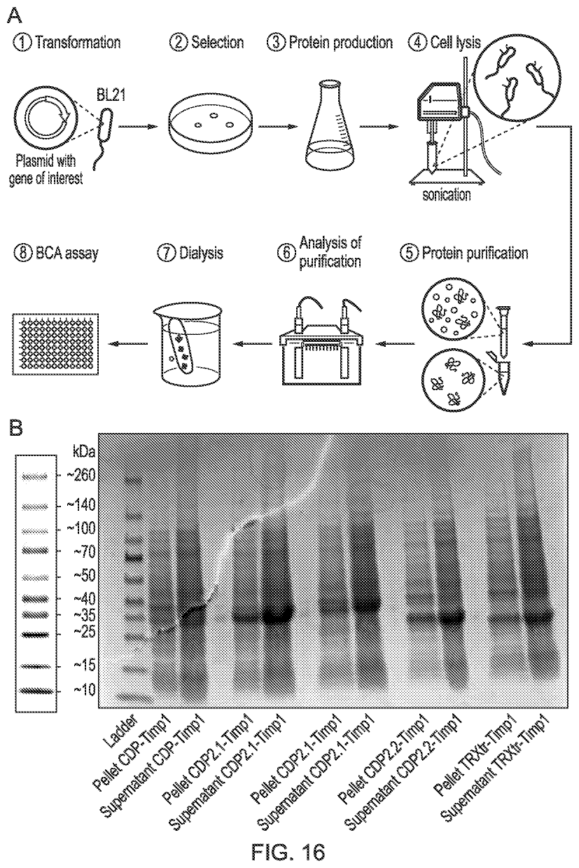

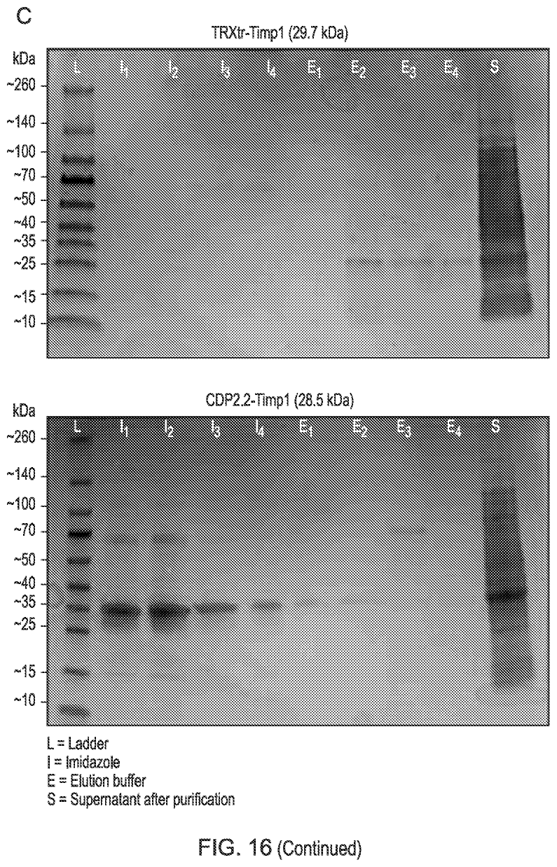

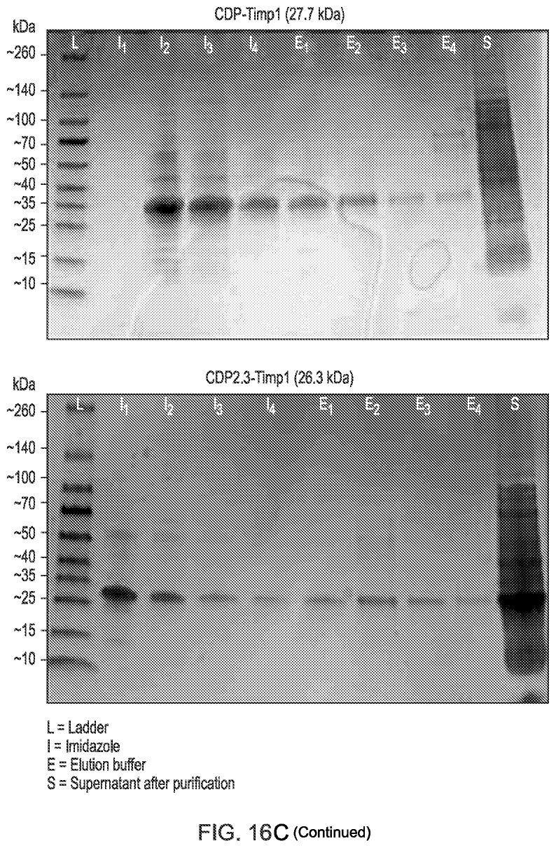

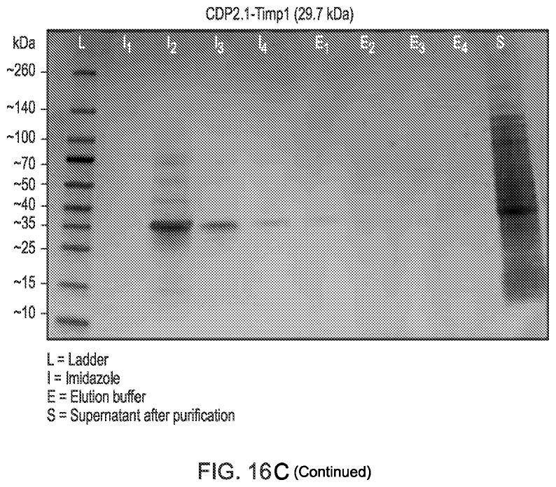

Protein production and purification of mouse Timp1 and mouse Timp1 fused to each of immune stimulating peptides: CDP (SEQ ID NO:5), CDP2.1 (SEQ ID NO:6), CDP2.2 (SEQ ID NO:7), CDP2.3 (SEQ ID NO:8) and TRXtr (SEQ ID NO:10) was carried out (FIG. 16A). After sonication, the pellet and the supernatant were loaded in an SDS-PAGE gel. Thick bands at the size of the fusion proteins were found in the supernatant (pointed out by the white arrows in FIG. 16B), and only a lower concentration of protein remained in the pellet. 5 mL supernatant was used with 200 μL of Ni-Agarose beads, resulting in a low concentration of TRXtr-Timp1 eluted in buffer E fractions, while the highest concentration of CDP-Timp1, CDP2.1-Timp1, CDP2.2-Timp1 and CDP2.3-Timp1 was eluted with imidazole fractions (FIG. 16C).

It was observed that conjugation of Timp1 to CDP or CDP2.1, CDP2.2. and CDP2.3 lowered the isoelectric point of the fusion protein thereby promoting purification with 200 mM imidazole. The table below summarises various properties of mouse Timp1, and mouse Timp1 fused to the C-terminus of each of TRXtr (SEQ ID NO:10), CDP (SEQ ID NO:5), CDP2.1 (SEQ ID NO: 6), CDP2.2 (SEQ ID NO:7) and CDP 2.3 (SEQ ID NO:8):

TABLE 2

| | MW | pl | Solubility |
|---|---|---|---|
| Timp1 | 21.3 kDa | 9.05 | 0.445 |
| TRXtr-Timp1 | 28.0 kDa | 9.30 | 0.475 |
| CDP-Timp1 | 27.7 kDa | 8.46 | 0.384 |
| CDP2.1-Timp1 | 29.7 kDa | 4.95 | 0.516 |
| CDP2.2-Timp1 | 28.6 kDa | 5.23 | 0.488 |
| CDP2.3-Timp1 | 26.3 kDa | 6.13 | 0.324 |

### Purification of Mammalian Peptide, Notum, Conjugated to Immune Stimulating Peptides, CDP (SEQ ID NO:5), CDP2.1 (SEQ ID NO:6), CDP2.2 (SEQ ID NO:7), CDP2.3 (SEQ ID NO:8) and TRXtr (SEQ ID NO:10)

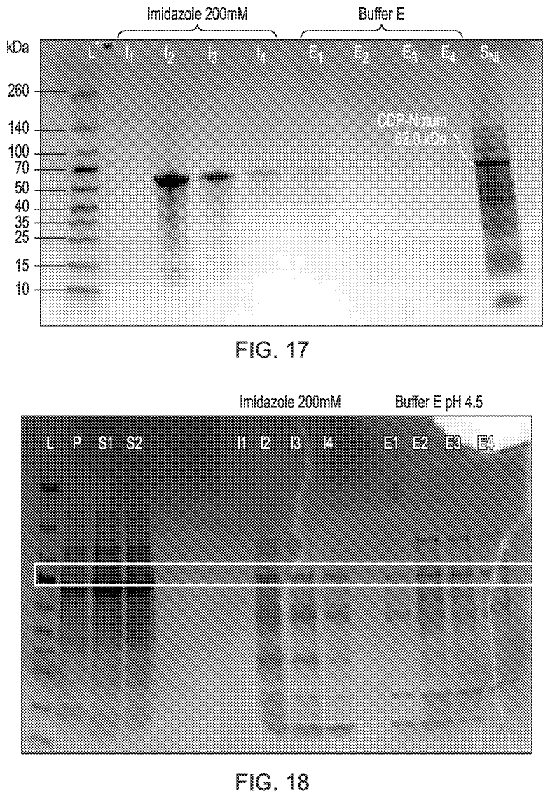

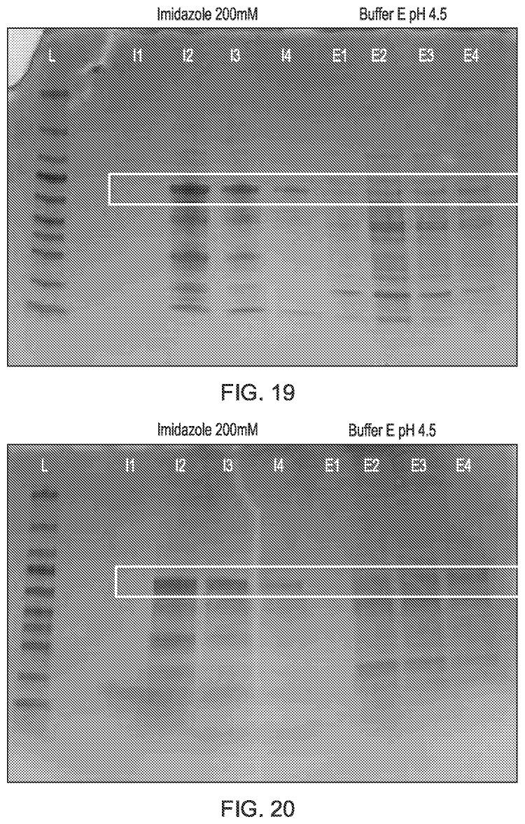

Fusion polypeptides of mouse Notum were fused to CDP (SEQ ID NO:5), CDP2.1 (SEQ ID NO:6), CDP2.2 (SEQ ID NO:7), CDP2.3 (SEQ ID NO:8) and TRXtr (SEQ ID NO:10) were successfully purified and eluted using 200 mM imidazole (FIG. 17).

The table below summarises various properties of mouse Notum, and mouse Notum fused to the C-terminus of each of TRXtr (SEQ ID NO:10), CDP (SEQ ID NO:5), CDP2.1 (SEQ ID NO: 6), CDP2.2 (SEQ ID NO:7) and CDP 2.3 (SEQ ID NO:8):

TABLE 3

| | MW | pl | Solubility |
|---|---|---|---|
| Notum | 55.7 kDa | 6.78 | 0.136 |
| TRXtr-Notum | 62.3 kDa | 7.90 | 0.141 |
| CDP-Notum | 62.0 kDa | 6.27 | 0.175 |
| CDP2.1-Notum | 64.1 kDa | 5.00 | 0.377 |
| CDP2.2-Notum | 62.9 kDa | 5.14 | 0.325 |
| CDP2.3-Notum | 60.5 kDa | 5.49 | 0.242 |

Purification of Influenza Peptides from Hemagglutinin (H) and/or Neuraminidase (N), Conjugated to Immune Stimulating Peptide, CDP (SEQ ID NO:5).

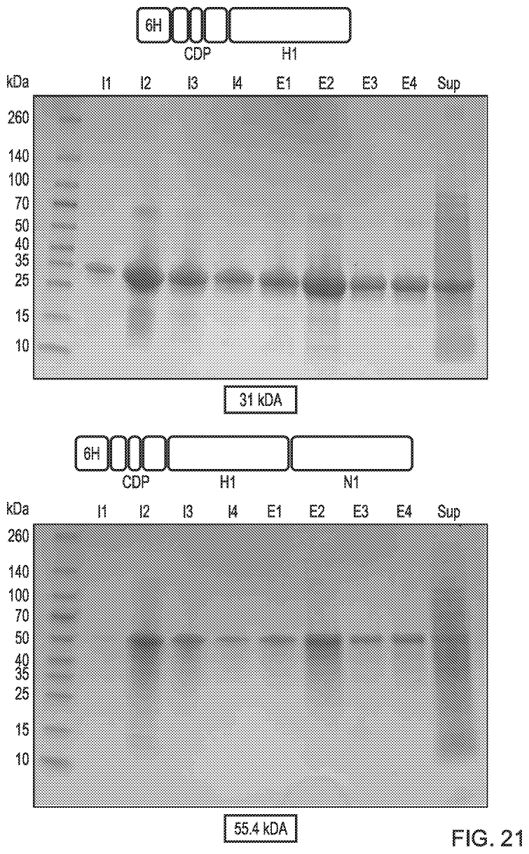

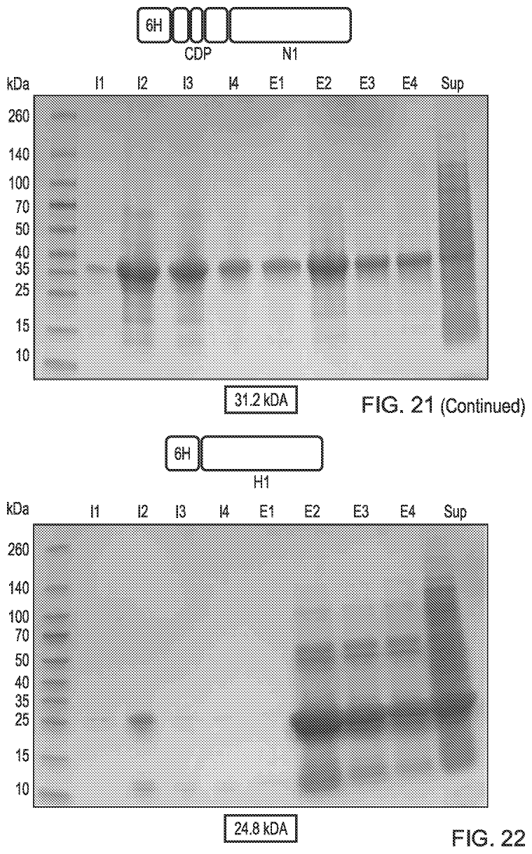

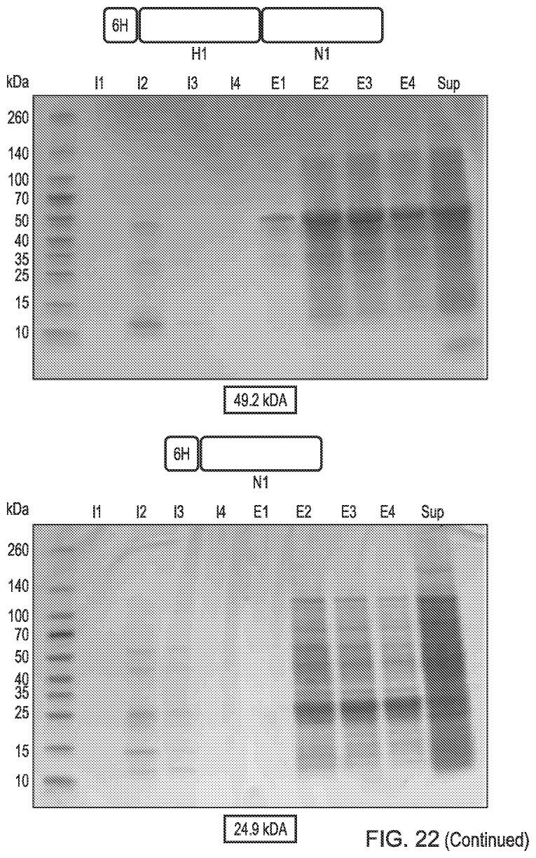

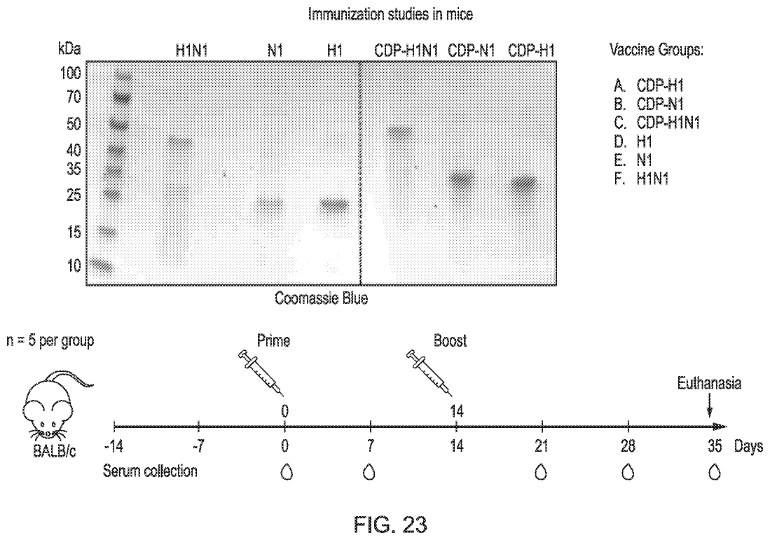

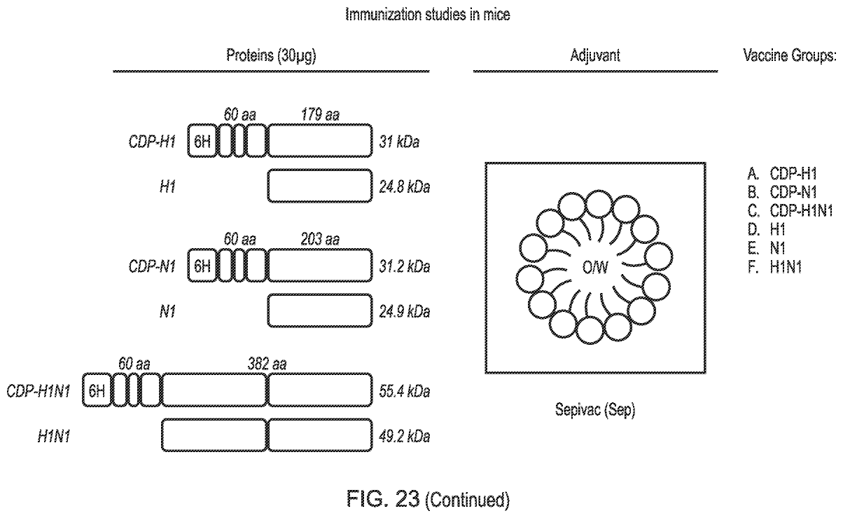

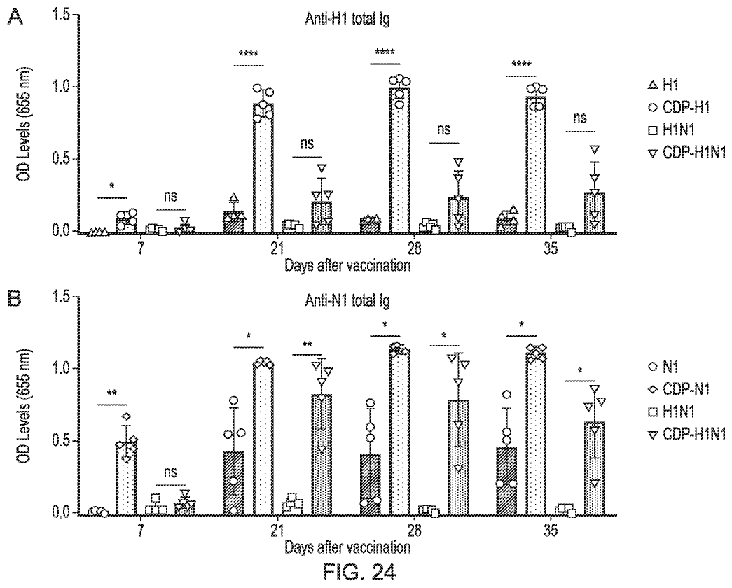

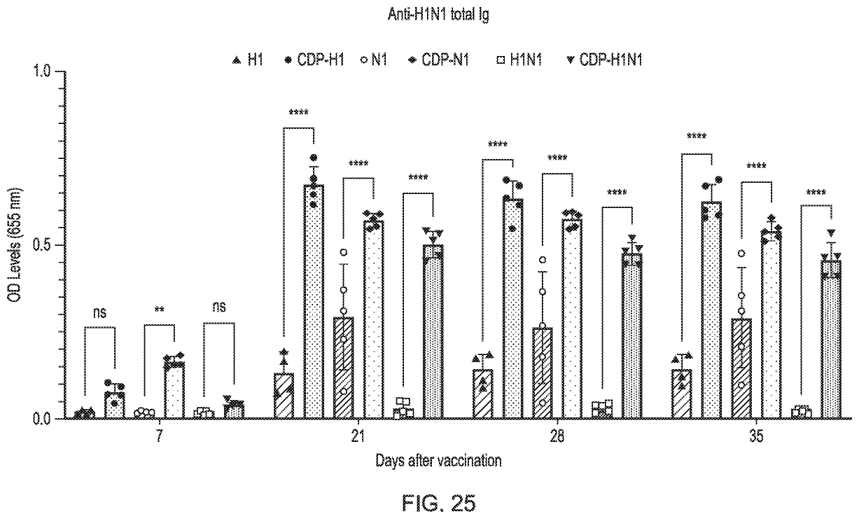

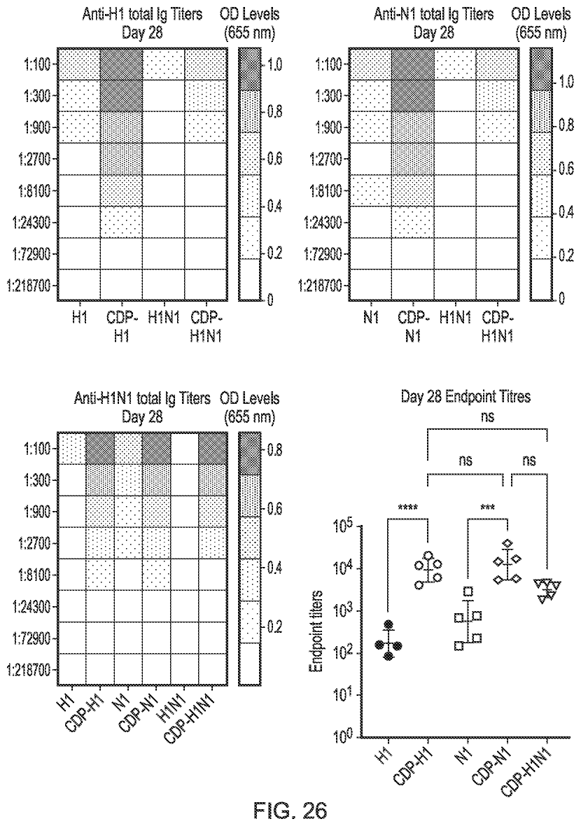

Data generated using these constructs is shown in FIGS. 21 to 26. Addition of CDP to the H1/N1 sequences lowers the isoelectric point of the protein and the protein can be eluted with imidazole (11-14). Whereas, unconjugated H1/N1 proteins can only be eluted with buffer E (E1-E4).

The table below summarises various properties of the influenza constructs, when fused to CDP (SEQ ID NO: 5):

TABLE 4

| | Mw | pl |
|---|---|---|
| H1N1 | 49.2 kDa | 7.62 |
| H1 | 24.9 kDa | 6.90 |
| N1 | 24.8 kDa | 8.34 |
| CDP-H1N1 | 55.4 kDa | 6.43 |
| CDP-H1 | 31.0 kDa | 6.05 |
| CDP-N1 | 31.2 kDa | 6.58 |

### Examples Section C

### Production of Vaccines for Non-Mammalian Peptides Conjugated to Immune Stimulating Peptide of SEQ ID NO:5 (CDP)

The inventors further designed, developed and characterized the vaccines disclosed herein exploiting the immune stimulating peptides of the invention in order to direct the immune response towards SARS-CoV-2, namely the accessible epitopes of the RBM (a small part of RBD, named receptor binding motif (RBM) which contains all the residues directly interacting with ACE-2), SD1, SD2, S1/S2, FP+S2′ and HR1 functional subdomains of the S (Spike) glycoprotein.

### Epitope Selection and Vaccine Production

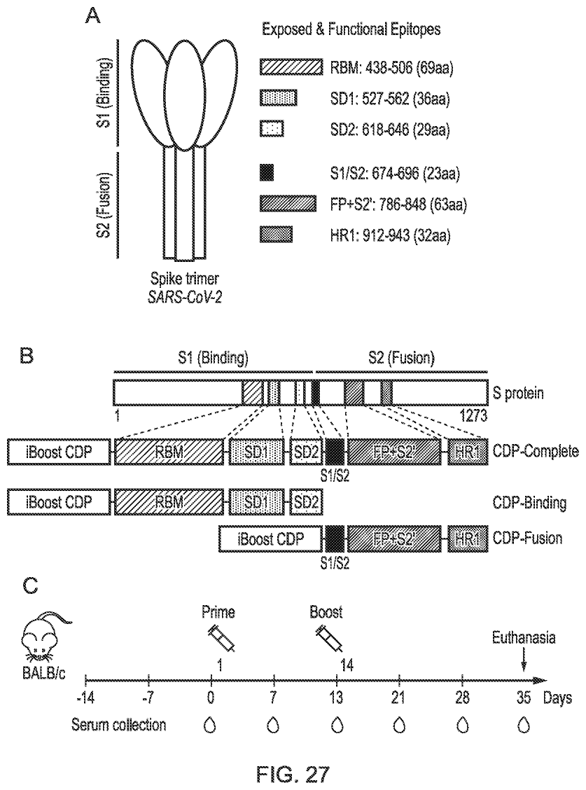

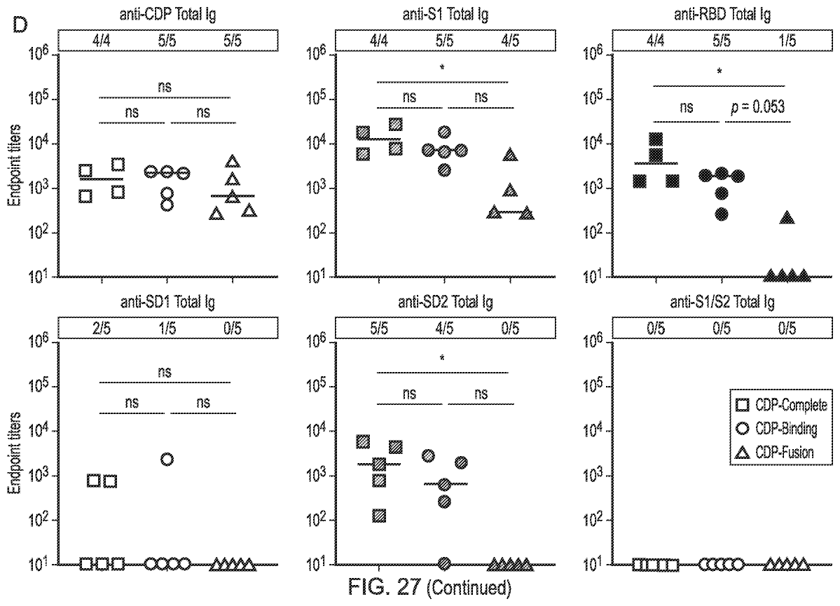

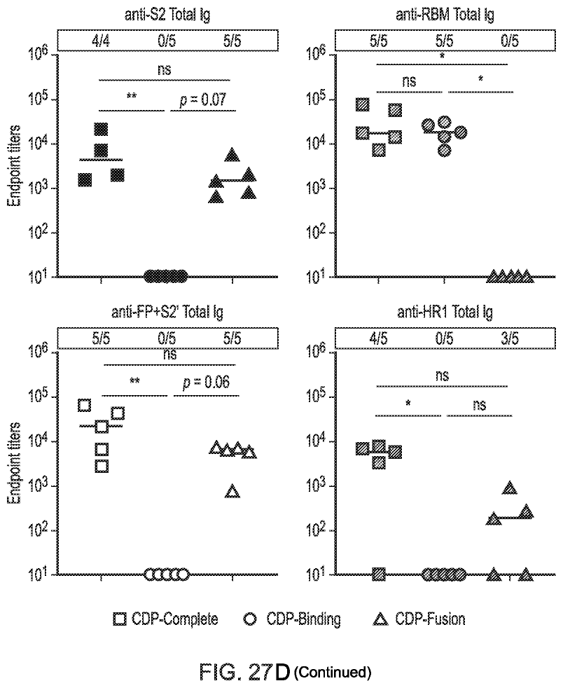

The S protein of SARS-CoV-2 was screened for functional domains based on the extensive work that has been provided by the global research community over the previous years. In total, six functional epitopes were identified, with three epitopes located in the S1 and three epitopes in the S2 subunit of the S protein (FIG. 27A). The first three selected epitopes are RBM, SD1 and SD2, playing an important role for host cell receptor ACE-2 recognition and binding (Henderson et al., 2020; Lan et al., 2020). The three selected epitopes located in the S2 domain are the S1/S2 cleavage site, FP and S2′ protease cleavage site (FP+S2′) and HR1, orchestrating the fusion machinery and viral entry into host cells. All six selected epitopes were further examined for their antibody accessibility. Only sections/parts within the selected epitopes that show a high antigen accessibility were selected for the generation of new vaccines (FIG. 27A).

The selected epitopes were linked with a flexible GSGSGS linker in the order mentioned above (RBM, SD1, SD2, S1/S2, FP+S2′, HR1) and the CDP sequence (SEQ ID NO:5) described elsewhere herein, was conjugated to the N terminus (FIG. 27B). This vaccine is further referred to as CDP-complete (FIG. 27B). In order to characterize the immunogenicity of the epitopes of the S1 and S2 subdomains, the inventors also generated vaccines, in which CDP is conjugated to either the three selected epitopes of the S1 subdomain (RBM, SD1, SD2) or to the three selected epitopes of the S2 subdomain (S1/S2, FP+S2′, HR1). These vaccines are referred to as CDP-binding and CDP-fusion, respectively (FIG. 27B).

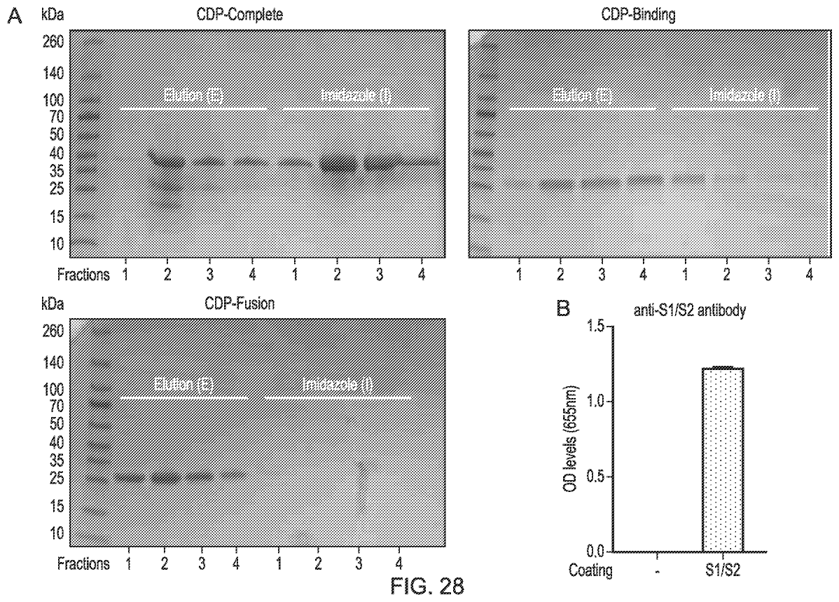

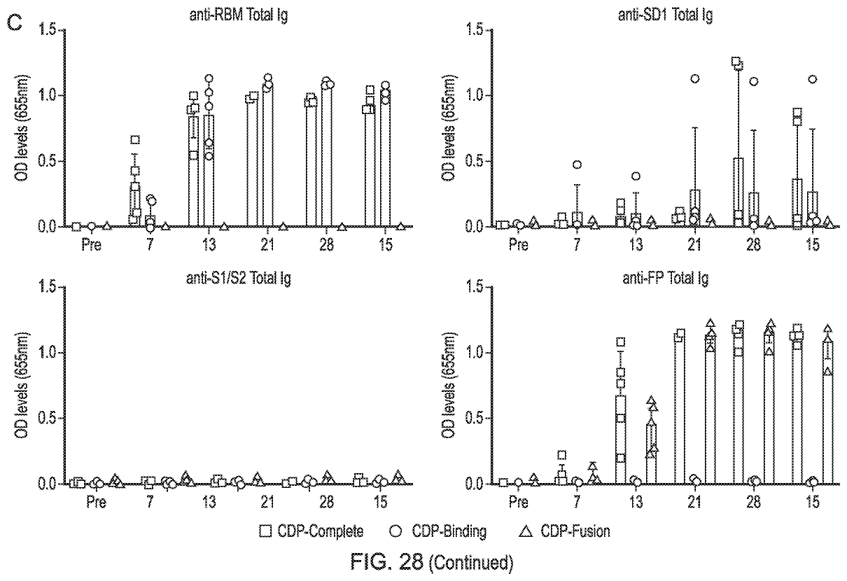

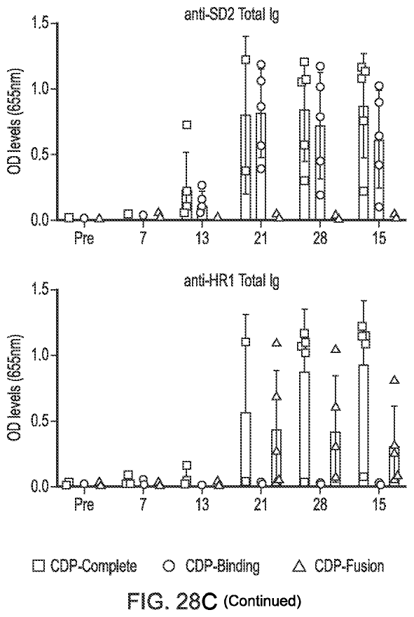

All three vaccines were expressed in E. coli BL21 and subsequently purified via immobilized metal chelate affinity chromatography (IMAC) as previously described (Huijbers et al., 2018) (Blanas et al., 2022). Colloidal Coomassie blue stained SDS-Page confirmed the purity and correct size of the produced vaccines (FIG. 28A).

### Induction of a Specific Humoral Response Against Selected Epitopes of the S Protein

It was first determined, whether the multi-epitope vaccine disclosed herein could simultaneously induce an immune response against multiple functional domains of the SARS-CoV-2 S protein. BALB/c mice were subcutaneously immunized with 100 μg CDP-complete vaccine protein mixed 1:1 (v/v) with Sepivac, an oil-in-water adjuvant. Injections were given at day 1 and 14 and blood samples were taken at time points (0, 7, 13, 21 28 and 35 days post immunization) to assess antibody titers via ELISA (FIG. 27C).

The CDP-complete vaccine elicited high total immunoglobulin (Ig) titers 35 days post prime immunization against the full length S1 and S2 subunits of the S protein (FIG. 27D). In more detail, all mice showed high antibody titers against RBD, RBM, SD2 as well as FP+S2′ and HR1 epitope. Only the SD1 epitope displayed a more diffuse antibody response with only two out of five mice showing specific antibody titers.

A similar antibody response against the epitopes of the S1 subunit was observed in mice immunized with the CDP-Binding vaccine (FIG. 27D). Again, high antibody titers for the S1 protein, RBD, RBM and SD2 were detected. The SD1 epitope triggered only antibodies in one out of five mice. As expected, immunization with CDP-Binding induced no antibodies against the S2 protein and its epitopes S1/S2, FP+S2′ or HR1.

The CDP-Fusion vaccine elicited high antibody titers against the S2 protein and the FP+S2′ epitope (FIG. 27D). Analysis of the HR1 epitope revealed that three out of five mice showed specific antibodies. Interestingly, a specific antibody response against the S1 subunit protein was also detected. Alignment of the sequences of the commercial full length S1 protein used for ELISA coating and the CDP-Fusion vaccine revealed a twelve amino acid overlap in the S1/S2 epitope (aa674-685), which may account for the antibody response.

Furthermore, all mice of the three vaccine groups elicited antibodies against the bacterial CDP domain. Interestingly, none of the mice showed S1/S2 epitope specific antibodies. This was not due to insufficient coating of the S1/S2 peptide, as the commercial anti-S1/S2 antibody showed strong signal, indicating coating of the S1/S2 peptide was efficient and that no antibodies were elicited after vaccination (FIG. 28B).

To unveil the kinetics of the antibody responses, sera of immunized mice between day 0 and day were analyzed at different time points and revealed high total Ig titers against RBM and FP+S2′ epitopes after only a single vaccination and 13 days post prime immunization. Specific antibodies were detected after two vaccinations and 21 days post prime immunization for SD2 and HR1 epitopes and 28 days post prime immunization for SD1 epitope (FIG. 28C).

These findings show for the first time that a multi epitope vaccine based on the technology disclosed herein can induce high antibody titers against several selected epitopes of the SARS-CoV-2 S protein.

### Induction of Systemic Epitope-Specific T-Cell Responses

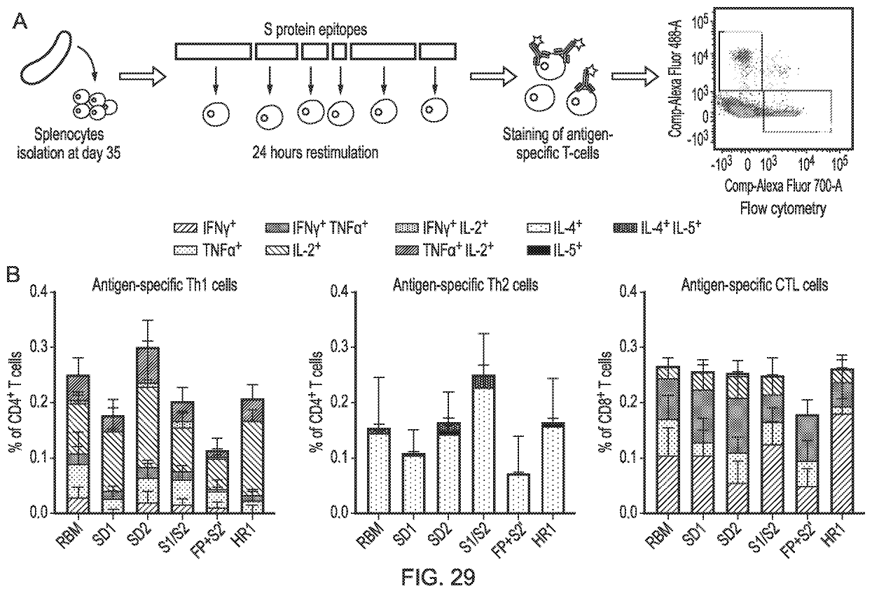

SARS-CoV-2-specific T cells are directly involved in the fast clearance of virally-infected cells and counteract development of mild disease symptoms. Therefore, the induction of potent antigen-specific T cell responses upon vaccination is of utmost importance. To investigate whether vaccination with CDP-complete elicits systemic T cell immunity against the selected functional epitopes of the S protein, the spleens of immunized mice were collected at Day 35. Splenocytes were re-stimulated ex vivo with the RBM, SD1, SD2, S1/S2, FP+S2′ and HR1 peptides and the frequency of antigen-specific T cells was quantified based on Th1- and Th2-specific cytokine production (FIG. 29A). Non-stimulated splenocytes were taken along as negative control.

Interestingly, mice developed multi-functional CD4⁺ and CD8⁺ T cells reactive to all the peptides examined after vaccination. In particular, antigen-specific T helper (Th) cells with distinct Th1 and Th2 cytokine profiles were detected (FIG. 29B). The Th1-skewed cells were predominantly characterized by interleukin 2 (IL-2) and tumor necrosis factor alpha (TNFα) production, whereas the Th2-skewed cells exhibited strong induction of IL-4. In contrast, CD8+ T cells induced after vaccination displayed a strong interferon gamma (IFNg) and TNFα profile, indicative of their potent cytotoxic potential. Based on these findings, vaccination with the conjugate CDP-Complete vaccine ensures generation of potent cellular responses targeting functional epitopes that did not induce a detectable antibody response, like the S1/S2 subdomain.

### CDP-Complete Vaccine in a SARS-CoV2 Hamster Challenge Model

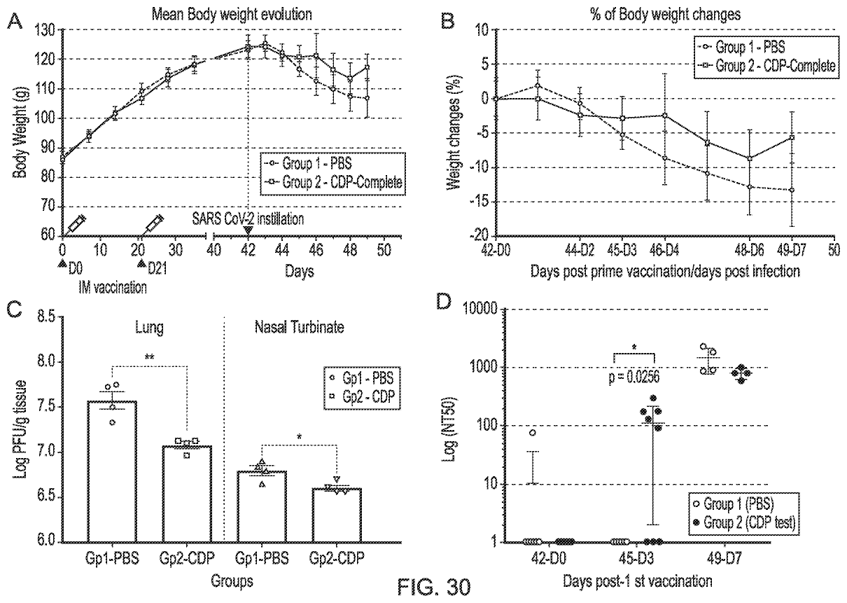

Hamsters (n=8 per group) were vaccinated twice (day 0 and day 21) with 100 I CDP-Complete vaccine, containing 100 g protein mixed 1:1 (v/v) with Sepivac adjuvant or PBS mixed with adjuvant (PBS control). Body weight of the hamsters was monitored during the experiment (FIG. 30A). On day 42 hamsters were challenged with SARS-CoV2 and body weight further monitored (FIG. 30B). Following SARS-CoV-2 infection of hamster, the BW of infected hamsters decreased from day 43 to day 49 to reach a decrease of about −13% for group 1 and -6% for group 2. In addition, for the second group, body weight loss was less rapid than for group 1. These results demonstrate that CDP-Complete seem to limit SARS-CoV-2 effect on body weight loss. Virus load in lung and nasal turbinate samples at D+3 post infection was measured (FIG. 30C). In the lungs and nasal turbinates of the CDP-complete vaccinated hamsters a significantly lower viral load was found. On day 45 (day 3 post viral challenge) 5/8 hamsters vaccinated with the CDP-Complete have developed neutralizing antibody responses, whereas no antibody responses were visible in the PBS control group (FIG. 30 D). On day 49 both groups have a detectable neutralizing antibody response against SARS-CoV2. Thus, upon SARS-CoV2 infection, animals immunized with the CDP-Complete vaccine developed a neutralizing antibody response earlier than the control group.

### Materials and Methods

### Vaccine Design

Each recombinant vaccine protein starts with a Hiss-tag (HHHHHH) followed by the iBoost CDP sequence at their N-terminus. CDP consists of bacterial amino acid clusters with hydrophilic or charged side chains that are fused into a single peptide with a total length of 60 amino acids (5.8 kDa). The selected SARS-CoV-2 S protein epitopes were linked with a (GS)₃-linker (GSGSGS) to CDP and to each other in the order as depicted in FIG. 27B. The sequences of the S protein epitopes are based on the reported SARS-CoV-2 genome (GenBank: MN908947.3) and are located: RBM: aa438-506 SD1: aa527-562, SD2: aa618-646, S1/S2: aa674-696, FP+S2′: aa786-848 and HR1: aa912-943.

### Vaccine Protein Production

The coding sequences of the recombinant vaccine proteins were synthesized by Genscript (USA) and cloned between the NdeI and XhoI restriction sites of the pET21a expression vector. Competent E. coli BL21 (DE3) bacteria (Sigma-Aldrich) were transformed with 10 ng plasmid DNA using the heat shock method. A single clone was selected and cultured in LB-medium supplemented with ampicillin (100 μg/ml) in a non-humified shaker (200 rpm, Laboshake) overnight at 37° C. The next day, the culture was 1:2.6-diluted and protein expression was induced with 1 mM Isopropyl β-d-1-thiogalactopyranoside (IPTG) (Serva) for four hours. Bacteria were harvested by centrifugation and bacterial pellets (equivalent to 50 ml culture volume) were dissolved in 5 ml sonication buffer containing 0.5 M EDTA (Sigma-Aldrich), 1% N-lauroylsarcosine (Sigma-Aldrich), 1% Phenylmethanesulfonyl fluoride solution (PMSF) (Sigma-Aldrich) and 6 M Urea (Acros Organics) in PBS (VWR). Subsequently, proteins were released by sonication (amplitude 22-24 microns) on ice with 15 cycles of 20 seconds sonication followed by 30 seconds break (Soniprep 150 MSE). Subsequently, samples were pelleted by centrifugation (4500 rpm, 20 min) and the supernatant, containing the proteins, was mixed with 200 pI 50% Ni-NTA agarose slurry (Qiagen). After overnight incubation on a roller bench at 4° C., beads were spun down and the supernatant frozen for additional purification rounds. Ni-NTA beads were washed five times with 25 mL wash buffer, containing 1 M NaCl, (VWR), 0.05% Tween-20 (Sigma-Aldrich) and PBS and afterwards stacked in a 1 mL syringe (BD Biosciences) with a glass filter (Sartorius). Vaccine proteins were eluted with four fractions (100 pI each) of 1 M Tris Cl (pH 8.0), 100 mM NaCl, 200 mM imidazole (JT Baker), 1 mM PMSF followed by four fractions (100 pI each) of 10 mM Tris Cl (pH 4.5), 100 mM NaH2PO4 (Merck), 8 M urea. Fractions of purified proteins were pooled and dialyzed stepwise towards PBS. SDS PAGE and Coomassie blue staining (Coomassie® Brilliant Blue G 250, Serva) confirmed the purity and identity of the vaccine proteins. The final protein concentration was determined by BCA assay (Thermo Scientific) and vaccines were diluted to a final concentration of 2 mg/ml.

### Mouse Experiments

Female 8 weeks-old BALB/c OlaHsd mice were obtained from Envigo (The Netherlands) and allowed to acclimatize two weeks at a temperature of 20-24° C., humidity of 45-65%, and a 12/12 hours light-dark cycle. After acclimatization mice received a prime vaccination on day 1 and a booster vaccination on day 14 (FIG. 27C). Each injection was done subcutaneously in the left groin with 100 μl of vaccine emulsion, consisting of 100 μg of purified vaccine protein mixed 50:50 (v/v) with Sepivac SWE (Seppic) adjuvant. Blood samples were taken from the tail vein prior to immunization (day 0) and on day 7, 13, 21, 28 and 35. At day 35 all mice were sacrificed. All mouse experiments were approved by the Dutch national ethics board Centrale Commissie Dierproeven (CCD, registration number AVD11400202010545) and the animal welfare body of the VU/Vumc (work protocol number 10545-ANG21-01) and were performed in agreement with Dutch guidelines and law on animal experimentation.

### ELISAs for Antibody Detection

The titers of total Ig binding to RBD, S1 subunit, S2 subunit or the selected epitopes (RBM, SD1, SD2, S1/S2, FP+S2′, HR1) in sera of immunized mice were measured by ELISA. Therefore, blood samples were allowed to coagulate overnight at 4° C. and centrifuged (7000 rpm, 10 min, 4° C.) twice the next day. Flat-bottom 96-well ELISA plates (Thermo Fisher Scientific) were coated with 50 μl of purified protein at a final concentration of 2 μg/mL (for RBD, S1, S2 subunit) or 4 μg/mL for selected S protein peptides in PBS at 37° C. for 1 hour. S protein peptides were ordered (Proteogenix, France) and reconstituted to a final concentration of 10 mg/ml according to the manufacturer's instructions. Next, plates were washed once with PBS containing 0.1% Tween-20 (PBS-T) and blocked with 1% non-fat dry milk (Santa Cruz) in PBS-T at 37° C. for 1 hour. After one wash, serial 3-fold dilutions (1/100 to 1/72900) of mouse sera in blocking solution were added to the plate, and incubated at 37° C. for 45 minutes. Plates were washed, and secondary biotinylated goat anti-mouse total Ig (Dako, #E0433) diluted in PBS-T (1/2000) was added to the wells and incubated at 37° C. for 45 minutes. After four washes, plates were incubated with Streptavidin-HRP (Dako, #P0397) diluted in PBS-T (1/2000) at 37° C. for 30 minutes. Plates were washed and developed with TMB (Sigma, #T0440) for 10 minutes. Absorbance was read at 655 nm on a microplate reader (BioTek Synergy HTX). Actual OD values were calculated by subtracting blank (wells that were treated like sample wells except adding blocking solution instead of mouse sera) values from sample values

### Splenocyte Isolation and Peptide Re-Stimulation

Spleens from mice immunized with CDP-Complete were isolated on day 35, cut into small pieces and mechanically dissociated. Splenocytes were passed through a 70 μm cell strainer (Corning, #431751) and spun down for 5 min at 1500 rpm (brake 5, room temperature) in a Rotina 420 R (Hettich) centrifuge. Red blood cells were lysed upon incubation with ammonium-chloride-potassium lysis buffer (150 mM NH₄Cl, 10 mM KHCO₃, 100 mM EDTA, pH=7.4) for 3 min at room temperature. Splenocytes were washed once and were cryopreserved in RPMI-1640 (Gibco, #L0495-500) medium containing 10% DMSO. Splenocytes were thawed and resuspended in RPMI-1640 medium supplemented with 10% FCS (Biowest, #S0750), 1% Pen/Strep (Gibco, #15140-122), 1% L-glutamine (Brunschwig Chemie BV, #HN08.2) and 50 μM 2-mercaptoethanol (Gibco, #31350-010). They were seeded in U-bottom 96-well plates (Greiner Bio-One, #650180) (2⁺ 10⁶ cells/well) and were restimulated ex vivo for 24 h with the RBM, SD1, SD2, S1/S2, FP+S2′ and HR1 synthesized peptides. Unstimulated splenocytes treated with Brefeldin A were taken along as a negative control.

### Flow Cytometric-Based Characterization of Epitope-Specific T Cells

After re-stimulation, cells were transferred to a V-bottom 96-well plate (Greiner Bio-One, #651180) and washed once with PBS. Cells were resuspended in TruStain Fc blocking solution (BioLegend, #101320) for 10 min at room temperature. Afterwards, cells were incubated with anti-mouse CD4-AF700 (BioLegend, #100429, 1/200 dilution), anti-mouse CD8b-AF488 (BioLegend, #126627, 1/200 dilution) and Zombie Aqua fixable viability dye (BioLegend, #423101, 1/200 dilution) diluted in PBS for 20 min on ice. Cells were washed once with PBS and fixed with 4% paraformaldehyde (PFA; Electron Microscopy Sciences, #15710) for 15 min on ice. After fixation, cells were washed once with PBS and permeabilized using the intracellular staining permeabilization wash buffer (BioLegend, #421002) following the manufacturer's instructions. Cell suspensions were then incubated with anti-mouse IFNγ-APC (BioLegend, #505305, 1/200 dilution), TNFα-PE (BioLegend, #506305, 1/200 dilution), IL-2-PE-Cy7 (BioLegend, #503832, 1/200 dilution), IL-4-PerCP/Cy5.5 (BioLegend, #504123, 1/200 dilution) and IL-5 (BioLegend, #504311, 1/200 dilution), diluted in intracellular staining permeabilization wash buffer for 30 min at room temperature. Cells were washed twice with the permeabilization wash buffer, resuspended in 100 pI PBS and transferred to FACS tubes. Fluorescence intensities and the percentage of IFNγ-, TNFα-, IL-2-, IL-4- and IL-5-expressing cells were measured using an LSRII (BD Biosciences) flow cytometer. The percentage of the no stimulation condition for each mouse was subtracted by the percentage of each stimulation condition. Data analysis was performed with the FlowJo V10 software (Tree Star).

### Hamster Housing

Hamsters were housed in type 2 cages with a maximum of two animals per cage under DM(BSL)-II conditions during the acclimatization and vaccination phase. Hamsters were transferred to standard elongated type 2 group cages with two animals per cage under BSL-Ill conditions (isolators) on the day of virus inoculation using sawdust as bedding with cage enrichment. For all invasive animal procedures, intranasal and intramuscular administration, blood sampling, throat swab collection and euthanasia, the animals were sedated with isoflurane (3-4%/O2). Hamsters were vaccinated according to the schedule via the intramuscular route (i.m) on day 0 and day 21. Blood was taken via orbital bleeding. Blood samples for serum were immediately transferred to appropriate tubes containing a clot activator. Serum was collected, heat treated, aliquoted and stored frozen.

### Hamster Vaccination

On day 0 and 21 animals were vaccinated with either PBS as control or 100 μg CDP-Complete mixed 1:1 (v/v) with Sepivac (Seppic) adjuvant in a total volume of 100 μL intramuscularly (i.m).

### SARS-CoV-2 Inoculation

On day 42, all hamsters were challenged with 50 μL of a SARS-CoV-2 WT variant (090621_SARS-CoV-2_UVE/SARS-CoV-2/2020/FR/702) suspension at a concentration of 2×106 PFU/mL was instilled, approximatively equally distributed into each nostril of hamster, using a thin pipette cone. On day 3 post challenge half of the animals per group were euthanized by exsanguination under isoflurane anesthesia and necropsy was performed. On day 7 post challenge, the remaining half of the animals per group were euthanized by exsanguination under isoflurane anesthesia and necropsy was performed.

### REFERENCES

Cohen, K. W., Linderman, S. L., Moodie, Z., Czartoski, J., Lai, L., Mantus, G., Norwood, C., Nyhoff, L. E., Edara, V. V., Floyd, K., et al. (2021). Longitudinal analysis shows durable and broad immune memory after SARS-CoV-2 infection with persisting antibody responses and memory B and T cells. Cell Rep Med 2, 100354. 10.1016/j.xcrm.2021.100354.
Gattinger, P., Niespodziana, K., Stiasny, K., Sahanic, S., Tulaeva, I., Borochova, K., Dorofeeva, Y., Schlederer, T., Sonnweber, T., Hofer, G., et al. (2022). Neutralization of SARS-CoV-2 requires antibodies against conformational receptor-binding domain epitopes. Allergy 77, 230-242. 10.1111/all.15066.
Henderson, R., Edwards, R. J., Mansouri, K., Janowska, K., Stalls, V., Gobeil, S. M. C., Kopp, M., Li, D., Parks, R., Hsu, A. L., et al. (2020). Controlling the SARS-CoV-2 spike glycoprotein conformation. Nat Struct Mol Biol 27, 925-933. 10.1038/s41594-020-0479-4.
Hoffmann, M., Kleine-Weber, H., Schroeder, S., Kruger, N., Herrler, T., Erichsen, S., Schiergens, T. S., Herrler, G., Wu, N. H., Nitsche, A., et al. (2020). SARS-CoV-2 Cell Entry Depends on ACE2 and TMPRSS2 and Is Blocked by a Clinically Proven Protease Inhibitor. Cell 181, 271-280 e278. 10.1016/j.cell.2020.02.052.
Huijbers, E. J. M., van Beijnum, J. R., Le, C. T., Langman, S., Nowak-Sliwinska, P., Mayo, K. H., and Griffioen, A. W. (2018). An improved conjugate vaccine technology; induction of antibody responses to the tumor vasculature. Vaccine 36, 3054-3060. 10.1016/j.vaccine.2018.03.064.
Koppisetti, R. K., Fulcher, Y. G., and Van Doren, S. R. (2021). Fusion Peptide of SARS-CoV-2 Spike Rearranges into a Wedge Inserted in Bilayered Micelles. J Am Chem Soc 143, 13205-13211. 10.1021/jacs.1c05435.
Lan, J., Ge, J., Yu, J., Shan, S., Zhou, H., Fan, S., Zhang, Q., Shi, X., Wang, Q., Zhang, L., and Wang, X. (2020). Structure of the SARS-CoV-2 spike receptor-binding domain bound to the ACE2 receptor. Nature 581, 215-220. 10.1038/s41586-020-2180-5.
Nelde, A., Bilich, T., Heitmann, J. S., Maringer, Y., Salih, H. R., Roerden, M., Lubke, M., Bauer, J., Rieth, J., Wacker, M., et al. (2021). SARS-CoV-2-derived peptides define heterologous and COVID-19-induced T cell recognition. Nat Immunol 22, 74-85. 10.1038/s41590-020-00808-x.
Niu, L., Wittrock, K. N., Clabaugh, G. C., Srivastava, V., and Cho, M. W. (2021). A Structural Landscape of Neutralizing Antibodies Against SARS-CoV-2 Receptor Binding Domain. Front Immunol 12, 647934. 10.3389/fimmu.2021.647934.
Perez-Potti, A., Lange, J., and Buggert, M. (2021). Deciphering the ins and outs of SARS-CoV-2-specific T cells. Nat Immunol 22, 8-9. 10.1038/s41590-020-00838-5.
Rabi, F. A., Al Zoubi, M. S., Kasasbeh, G. A., Salameh, D. M., and Al-Nasser, A. D. (2020). SARS-CoV-2 and Coronavirus Disease 2019: What We Know So Far. Pathogens 9. 10.3390/pathogens9030231.
Rossler, A., Riepler, L., Bante, D., von Laer, D., and Kimpel, J. (2022). SARS-CoV-2 Omicron Variant Neutralization in Serum from Vaccinated and Convalescent Persons. N Engl J Med 386, 698-700. 10.1056/NEJMc2119236.
Tenforde, M. W., Self, W. H., Adams, K., Gaglani, M., Ginde, A. A., McNeal, T., Ghamande, S., Douin, D. J., Talbot, H. K., Casey, J. D., et al. (2021). Association Between mRNA Vaccination and COVID-19 Hospitalization and Disease Severity. JAMA 326, 2043-2054. 10.1001/jama.2021.19499.
van Beijnum, J. R., Huijbers, E. J. M., van Loon, K., Blanas, A., Akbari, P., Roos, A., Wong, T. J., Denisov, S. S., Hackeng, T. M., Jimenez, C. R., et al. (2022). Extracellular vimentin mimics VEGF and is a target for anti-angiogenic immunotherapy. Nat Commun 13, 2842. 10.1038/s41467-022-30063-7.
Xia, S., Zhu, Y., Liu, M., Lan, Q., Xu, W., Wu, Y., Ying, T., Liu, S., Shi, Z., Jiang, S., and Lu, L. (2020). Fusion mechanism of 2019-nCoV and fusion inhibitors targeting HR1 domain in spike protein. Cell Mol Immunol 17, 765-767. 10.1038/s41423-020-0374-2.
Zhao, P., Praissman, J. L., Grant, O. C., Cai, Y., Xiao, T., Rosenbalm, K. E., Aoki, K., Kellman, B. P., Bridger, R., Barouch, D. H., et al. (2020). Virus-Receptor Interactions of Glycosylated SARS-CoV-2 Spike and Human ACE2 Receptor. bioRxiv. 10.1101/2020.06.25.172403.

SEQUENCES
Amino acids 330 to 524 of the receptor binding
domain (RBD) of SARS-COV-2 (GenBank: MN908947.3)
sequence (SEQ ID NO: 1):
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRK
SNLKPFERDISTEIYQAGSTPCNGVEGFNCYFPLQSYGFQPTNGVGYQPYRVVVLSFELLHAPA
TV
CD4 T-cell epitope construct 1 sequence of MsyB
protein from E. coli (SEQ ID NO: 2):
NPGIDAEDANVQQFNAQKYVLQDGDIMWQV
CD4 T-cell epitope construct 2 sequence of MsyB
protein from E. coli (SEQ ID NO: 3):
EGEFQLEPPLDTEEGRAAADE
Sequence of RRAB protein from E. coli (SEQ ID NO: 4):
DDEDFVDEDDD
Immune stimulating peptide sequence [original
CDP] (SEQ ID NO: 5):
DNNSLSQEVQNGSNHLENNQSQSNGGGSDSALSLSSKTAALAAATTVNDGSDGATSSAVG
Immune stimulating peptide sequence [IDP1 or
CDP2.1] (SEQ ID NO: 6);
HHAHTMYATLEEAIDAAREEFLADNPGIDAEDANVQQFNAQKYVLQDGDIMWQVEFFADEGEEG
EDDEDFVDEDDD
Immune stimulating peptide sequence [IDP2 or CDP
2.2] (SEQ ID NO: 7);
HHAHNPGIDAEDANVQQFNAQKYVLQDGDIMWQVEGEFQLEPPLDTEEGRAAADEDDEDFVDED
DD
Immune stimulating peptide sequence [IDP3 or
CDP2.3] (SEQ ID NO: 8):
HHAHNPGIDAEDANVQQFNAQKYVLQDGDIMWQVDDEDFVDEDDD
Thioredoxin (TRX) sequence (SEQ ID NO: 9);
SDKIIHLTDDSFDTDVLKADGAILVDFWAEWCGPCKMIAPILDEIADEYQGKLTVAKLNIDQNP
GTAPKYGIRGIPTLLLFKNGEVAATKVGALSKGQLKEFLDANLA
Thioredoxin truncated (TRXtrunc) sequence
(SEQ ID NO: 10):
GKLTVAKLNIDQNPGTAPKYGIRGIPTLLLFKNGEVAATKVGALSKGQLKEFLDANLA
Type-1 fimbrial protein, A chain sequence
(SEQ ID NO: 11);
AATTVNGGTVHFKGEVVNAACAVDAGSVDQTVQLGQVRTASLAQEGATSSAVGFNIQLNDCDTN
VASKAAVAFLGTAIDAGHTNVLALQSSAAGSATNVGVQILDRTGAALTLDGATFSSETTLNNGT
NTI
Variant Type-1 fimbrial protein, A chain
sequence (SEQ ID NO: 12):
KATTVNGGTVHFKGEVVNAACAVDAGSVDQTVQLGQVRTASLAQEGATSSKVGFNIQLNDCDTN
VASKAAVAFLGTKIDAGHTNVLALQSSAAGSDTNVGVQILDRTGAALTLDGATFSSETTLNNDT
NTI
CD4 T-cell epitope 1 sequence of RRAB protein
from E. coli (SEQ ID NO: 13):
KLGYEVYEVTDPEELEVE
CD4 T-cell epitope 1 sequence of RRAB protein
from E. coli (SEQ ID NO: 14):
FKLGYEVYEVTDPEELEV
CD4 T-cell epitope 1 sequence of RRAB protein
from E. coli (SEQ ID NO: 15):
YEVTDPEEL
CD4 T-cell epitope sequence of MsyB protein
from E. coli (SEQ ID NO: 16):
EGEFQLEPPLDTEEG
CD4 T-cell epitope sequence of MsyB protein
from E. coli (SEQ ID NO: 17):
AQKYVLQDGDIMWQV
CD4 T-cell epitope sequence of MsyB protein
from E. coli (SEQ ID NO: 18):
DEGEFQLEPPLDTEE
CD4 T-cell epitope sequence of MsyB protein
from E. coli (SEQ ID NO: 19):
NAQKYVLQDGDIMWQ
CD4 T-cell epitope sequence of MsyB protein
from E. coli (SEQ ID NO: 20):
EPPLDTEEGRAADE
CD4 T-cell epitope sequence of MsyB protein
from E. coli (SEQ ID NO: 21):
NPGIDAEDANVQQFN
CD4 T-cell epitope sequence of MsyB protein
from E. coli (SEQ ID NO: 22):
LEPPLDTEEGRAAD
CD4 T-cell epitope sequence of MsyB protein
from E. coli (SEQ ID NO: 23):
FQLEPPLDTYVLQDGDIM
CD4 T-cell epitope sequence of MsyB protein
from E. coli (SEQ ID NO: 24):
LDTEEGRAA
CD4 T-cell epitope sequence of MsyB protein
from E. coli (SEQ ID NO: 25):
IDAEDANVQ
His tag (SEQ ID NO: 26):
HHHHHH
Linker (SEQ ID NO: 27);
GSGSGS
ZapB (amino acids 36-45) (SEQ ID NO: 28);
NNSLSQEVQN
IbpA (amino acids 21-33) (SEQ ID NO: 29);
NHLENNQSQSNGG
TFP sequence (amino acids 12-39) (SEQ ID NO: 30):
SALSLSSTAALAAATTVN
TFP sequence (amino acids 69-76) (SEQ ID NO: 31);
GATSSAVG
TRXtrunc- Vimentin protein sequence (540 aa)
(linker and his tag underlined, vimentin
sequence in bold) (SEQ ID NO: 34);
MGKLTVAKLNIDQNPGTAPKYGIRGIPTLLLFKNGEVAATKVGALSKGQLKEFLDANLAGSGSG
SGSSTRSVSSSSYRRMFGGSGTSSRPSSNRSYVTTSTRTYSLGSALRPSTSRSLYSSSPGGAYV
TRSSAVRLRSSVPGVRLLQDSVDFSLADAINTEFKNTRTNEKVELQELNDRFANYIDKVRFLEQ
QNKILLAELEQLKGQGKSRLGDLYEEEMRELRRQVDQLTNDKARVEVERDNLAEDIMRLREKLQ
EEMLQREEAESTLQSFRQDVDNASLARLDLERKVESLQEEIAFLKKLHDEEIQELQAQIQEQHV
QIDVDVSKPDLTAALRDVRQQYESVAAKNLQEAEEWYKSKFADLSEAANRNNDALRQAKQESNE
YRRQVQSLTCEVDALKGTNESLERQMREMEENFALEAANYQDTIGRLQDEIQNMKEEMARHLRE
YQDLLNVKMALDIEIATYRKLLEGEESRISLPLPTFSSLNLRETNLESLPLVDTHSKRTLLIKT
VETRDGQVINETSQHHDDLERSHHHHHH
CDP-mVimentin protein (542 aa) (linker and
his tag underlined, vimentin sequence in bold)
(SEQ ID NO: 35):
MDNNSLSQEVQNGSNHLENNQSQSNGGGSDSALSLSSKTAALAAATTVNDGSDGATSSAVGGSG
SGSGSSTRSVSSSSYRRMFGGSGTSSRPSSNRSYVTTSTRTYSLGSALRPSTSRSLYSSSPGGA
YVTRSSAVRLRSSVPGVRLLQDSVDFSLADAINTEFKNTRTNEKVELQELNDRFANYIDKVRFL
EQQNKILLAELEQLKGQGKSRLGDLYEEEMRELRRQVDQLTNDKARVEVERDNLAEDIMRLREK
LQEEMLQREEAESTLQSFRQDVDNASLARLDLERKVESLQEEIAFLKKLHDEEIQELQAQIQEQ
HVQIDVDVSKPDLTAALRDVRQQYESVAAKNLQEAEEWYKSKFADLSEAANRNNDALRQAKQES
NEYRRQVQSLTCEVDALKGTNESLERQMREMEENFALEAANYQDTIGRLQDEIQNMKEEMARHL
REYQDLLNVKMALDIEIATYRKLLEGEESRISLPLPTFSSLNLRETNLESLPLVDTHSKRTLLI
KTVETRDGQVINETSQHHDDLEEFHHHHHH
IDP1-mVimentin protein (linker underlined,
vimentin sequence in bold) (SEQ ID NO: 36):
MHHAHTMYATLEEAIDAAREEFLADNPGIDAEDANVQQFNAQKYVLQDGDIMWQVEFFADEGEE
GEDDEDFVDEDDDGSGSGSGSSTRSVSSSSYRRMFGGSGTSSRPSSNRSYVTTSTRTYSLGSAL
RPSTSRSLYSSSPGGAYVTRSSAVRLRSSVPGVRLLQDSVDFSLADAINTEFKNTRTNEKVELQ
ELNDRFANYIDKVRFLEQQNKILLAELEQLKGQGKSRLGDLYEEEMRELRRQVDQLTNDKARVE
VERDNLAEDIMRLREKLQEEMLQREEAESTLQSFRQDVDNASLARLDLERKVESLQEEIAFLKK
LHDEEIQELQAQIQEQHVQIDVDVSKPDLTAALRDVRQQYESVAAKNLQEAEEWYKSKFADLSE
AANRNNDALRQAKQESNEYRRQVQSLTCEVDALKGTNESLERQMREMEENFALEAANYQDTIGR
LQDEIQNMKEEMARHLREYQDLLNVKMALDIEIATYRKLLEGEESRISLPLPTFSSLNLRETNL
ESLPLVDTHSKRTLLIKTVETRDGQVINETSQHHDDLE
IDP2-mVimentin protein (linker underlined,
vimentin sequence in bold) (SEQ ID NO: 37);
MHHAHNPGIDAEDANVQQFNAQKYVLQDGDIMWQVEGEFQLEPPLDTEEGRAAADEDDEDFVDE
DDDGSGSGSGSSTRSVSSSSYRRMFGGSGTSSRPSSNRSYVTTSTRTYSLGSALRPSTSRSLYS
SSPGGAYVTRSSAVRLRSSVPGVRLLQDSVDFSLADAINTEFKNTRTNEKVELQELNDRFANYI
DKVRFLEQQNKILLAELEQLKGQGKSRLGDLYEEEMRELRRQVDQLTNDKARVEVERDNLAEDI
MRLREKLQEEMLQREEAESTLQSFRQDVDNASLARLDLERKVESLQEEIAFLKKLHDEEIQELQ
AQIQEQHVQIDVDVSKPDLTAALRDVRQQYESVAAKNLQEAEEWYKSKFADLSEAANRNNDALR
QAKQESNEYRRQVQSLTCEVDALKGTNESLERQMREMEENFALEAANYQDTIGRLQDEIQNMKE
EMARHLREYQDLLNVKMALDIEIATYRKLLEGEESRISLPLPTFSSLNLRETNLESLPLVDTHS
KRTLLIKTVETRDGQVINETSQHHDDLE
IDP3-mVimentin protein (linker underlined,
vimentin sequence in bold) (SEQ ID NO: 38);
MHHAHNPGIDAEDANVQQFNAQKYVLQDGDIMWQVDDEDFVDEDDDGSGSGSGSSTRSVSSSSY
RRMFGGSGTSSRPSSNRSYVTTSTRTYSLGSALRPSTSRSLYSSSPGGAYVTRSSAVRLRSSVP
GVRLLQDSVDFSLADAINTEFKNTRTNEKVELQELNDRFANYIDKVRFLEQQNKILLAELEQLK
GQGKSRLGDLYEEEMRELRRQVDQLTNDKARVEVERDNLAEDIMRLREKLQEEMLQREEAESTL
QSFRQDVDNASLARLDLERKVESLQEEIAFLKKLHDEEIQELQAQIQEQHVQIDVDVSKPDLTA
ALRDVRQQYESVAAKNLQEAEEWYKSKFADLSEAANRNNDALRQAKQESNEYRRQVQSLTCEVD
ALKGTNESLERQMREMEENFALEAANYQDTIGRLQDEIQNMKEEMARHLREYQDLLNVKMALDI
EIATYRKLLEGEESRISLPLPTFSSLNLRETNLESLPLVDTHSKRTLLIKTVETRDGQVINETS
QHHDDLE
SARS-COV-2 sequence used in “CDP-complete”
(RBM-GS-linker-SD1-GS-linker-SD2-GS-linker-
S1/S2-GS-linker-FP-GS-linker-HR1 - linkers
underlined and SARS-COV-2 sequences in italics
(SEQ ID NO: 39)
SNNLDSKVGGNYNYLYRLFRKSNLKPFERDISTEIYQAGSTPCNGVEGFNCYFPLQSYGFQPTN
GVGYQGSGSGSPKKSTNLVKNKCVNFNFNGLTGTGVLTESNKKFLPFGSGSGSTEVPVAIHADQ
LTPTWRVYSTGSNVFQTRGSGSGSYQTQTNSPRRARSVASQSIIAYTGSGSGSKQIYKTPPIKD
FGGFNFSQILPDPSKPSKRSFIEDLLFNKVTLADAGFIKQYGDCLGDIAARDGSGSGSTQNVLY
ENQKLIANQFNSAIGKIQDSLSSTAS
The SARS-COV-2 sequence used in “CDP-binding”
(RBM-GS-linker-SD1-GS-linker-SD2 - linkers
underlined, SARS-COV2 sequences in italics)
(SEQ ID NO: 40)
SNNLDSKVGGNYNYLYRLFRKSNLKPFERDISTEIYQAGSTPCNGVEGFNCYFPLQSYGFQPT
NGVGYQGSGSGSPKKSTNLVKNKCVNFNFNGLTGTGVLTESNKKFLPFGSGSGSTEVPVAIHA
DQLTPTWRVYSTGSNVFQTR
The SARS-COV-2 sequence used in “CDP-fusion”
(S1/S2-GS-linker-FP-GS-linker-HR1 protein
sequence - linkers underlined, SARS-COV-2
sequences in italics) (SEQ ID NO: 41)
YQTQTNSPRRARSVASQSIIAYTGSGSGSKQIYKTPPIKDFGGFNFSQILPDPSKPSKRSFIED
LLFNKVTLADAGFIKQYGDCLGDIAARDGSGSGSTQNVLYENQKLIANQFNSAIGKIQDSLSST
AS
HI (SEQ ID NO: 42)
CNIAGWILGNPECESLSTARSWSYIVETSNSDNGTCYPGDFINYEELREQLSSVSSFERFEIFP
KTSSWPNHDSDNGVTAACPHAGAKSFYKNLIWLVKKGKSYPKINQTYINDKGKEVLVLWGIHHP
PTIADQQSLYQNADAYVFVGTSRYSKKFKPEIATRPKVRDQEGRMNYYWTLVEPGDKITFEATG
NLVAPRYAFTMERDAG
N1 (SEQ ID NO: 43)
WLTIGISGPDSGAVAVLKYNGIITDTIKSWRNKILRTQESECACVNGSCFTIMTDGPSDGQASY
KIFRIEKGKIIKSVEMKAPNYHYEECSCYPDSSEITCVCRDNWHGSNRPWVSFNQNLEYQMGYI
CSGVFGDNPRPNDKTGSCGPVSSNGANGVKGFSFKYGNGVWIGRTKSISSRKGFEMIWDPNGWT
GTDNKFSKKQDIVGINEWSGYS
H1/N1 (SEQ ID NO: 44)
CNIAGWILGNPECESLSTARSWSYIVETSNSDNGTCYPGDFINYEELREQLSSVSSFERFEIFP
KTSSWPNHDSDNGVTAACPHAGAKSFYKNLIWLVKKGKSYPKINQTYINDKGKEVLVLWGIHHP
PTIADQQSLYQNADAYVFVGTSRYSKKFKPEIATRPKVRDQEGRMNYYWTLVEPGDKITFEATG
NLVAPRYAFTMERDAGGSGSGSELWLTIGISGPDSGAVAVLKYNGIITDTIKSWRNKILRTQES
ECACVNGSCFTIMTDGPSDGQASYKIFRIEKGKIIKSVEMKAPNYHYEECSCYPDSSEITCVCR
DNWHGSNRPWVSFNQNLEYQMGYICSGVFGDNPRPNDKTGSCGPVSSNGANGVKGFSFKYGNGV
WIGRTKSISSRKGFEMIWDPNGWTGTDNKFSKKQDIVGINEWSGYS
CDP - H1 (CDP sequence in bold, linker
underlined, H1 sequence in italics)
(SEQ ID NO: 45)
HMHHHHHHDNNSLSQEVQNGSNHLENNQSQSNGGGSDSALSLSSKTAALAAATTVNDGSDGATS
SAVGGSGSGSGSCNIAGWILGNPECESLSTARSWSYIVETSNSDNGTCYPGDFINYEELREQLS
SVSSFERFEIFPKTSSWPNHDSDNGVTAACPHAGAKSFYKNLIWLVKKGKSYPKINQTYINDKG
KEVLVLWGIHHPPTIADQQSLYQNADAYVFVGTSRYSKKFKPEIATRPKVRDQEGRMNYYWTLV
EPGDKITFEATGNLVAPRYAFTMERDAG
Number of amino acids: 284
Molecular weight: 31030.97 (31.0 kDa)
Theoretical pl: 6.05
CDP - N1 (CDP sequence in bold, linker
underlined, N1 sequence in italics)
(SEQ ID NO: 46)
HMHHHHHHDNNSLSQEVQNGSNHLENNQSQSNGGGSDSALSLSSKTAALAAATTVNDGSDGATS
SAVGGSGSGSGSWLTIGISGPDSGAVAVLKYNGIITDTIKSWRNKILRTQESECACVNGSCFTI
MTDGPSDGQASYKIFRIEKGKIIKSVEMKAPNYHYEECSCYPDSSEITCVCRDNWHGSNRPWVS
FNQNLEYQMGYICSGVFGDNPRPNDKTGSCGPVSSNGANGVKGFSFKYGNGVWIGRTKSISSRK
GFEMIWDPNGWTGTDNKFSKKQDIVGINEWSGYS
Number of amino acids: 290
Molecular weight: 31158.15 (31.2 kDa)
Theoretical pl: 6.58
CDP - H1N1 (CDP sequence in bold, linker
underlined, H1N1 sequence in italics)
(SEQ ID NO: 47)
HMHHHHHHDNNSLSQEVQNGSNHLENNQSQSNGGGSDSALSLSSKTAALAAATTVNDGSDGATS
SAVGGSGSGSGSCNIAGWILGNPECESLSTARSWSYIVETSNSDNGTCYPGDFINYEELREQLS
SVSSFERFEIFPKTSSWPNHDSDNGVTAACPHAGAKSFYKNLIWLVKKGKSYPKINQTYINDKG
KEVLVLWGIHHPPTIADQQSLYQNADAYVFVGTSRYSKKFKPEIATRPKVRDQEGRMNYYWTLV
EPGDKITFEATGNLVAPRYAFTMERDAGGSGSGSELWLTIGISGPDSGAVAVLKYNGIITDTIK
SWRNKILRTQESECACVNGSCFTIMTDGPSDGQASYKIFRIEKGKIIKSVEMKAPNYHYEECSC
YPDSSEITCVCRDNWHGSNRPWVSFNQNLEYQMGYICSGVFGDNPRPNDKTGSCGPVSSNGANG
VKGFSFKYGNGVWIGRTKSISSRKGFEMIWDPNGWTGTDNKFSKKQDIVGINEWSGYS
Number of amino acids: 506,
Molecular weight: 55401.19 (55.4 kDa)
Theoretical pl: 6.42
His-TRXtr-mApelin protein (TRXtr sequence in
bold, linker underlined, mApelin in italics)
(SEQ ID NO: 48)
MHHHHHHGKLTVAKLNIDQNPGTAPKYGIRGIPTLLLFKNGEVAATKVGALSKGQLKEFLDANL
AGSGSGSGSNLRLCVQALLLLWLSLTAVCGVPLMLPPDGTGLEEGSMRYLVKPRTSRTGPGAWQ
GGRRKFRRQRPRLSHKGPMPF
Number of amino acids: 149
Molecular weight: 16104.81 Da (16.1 kDa)
Theoretical pl: 10.80
His-CDP-mApelin protein (CDP sequence in bold,
linker underlined, mApelin in italics)
(SEQ ID NO: 49)
MHHHHHHDNNSLSQEVQNGSNHLENNQSQSNGGGSDSALSLSSKTAALAAATTVNDGSDGATSS
AVGGSGSGSGSNLRLCVQALLLLWLSLTAVCGVPLMLPPDGTGLEEGSMRYLVKPRTSRTGPGA
WQGGRRKFRRQRPRLSHKGPMPF
Number of amino acids: 151
Molecular weight: 15834.59 Da (15.8 kDa)
Theoretical pl: 9.88
CDP2.1-Apelin (CDP2.1/IDP1 sequence in bold,
linker underlined, Apelin in italics)
(SEQ ID NO: 50)
MHHAHTMYATLEEAIDAAREEFLADNPGIDAEDANVQQFNAQKYVLQDGDIMWQVEFFADEGEE
GEDDEDFVDEDDDGSGSGSGSNLRLCVQALLLLWLSLTAVCGVPLMLPPDGTGLEEGSMRYLVK
PRTSRTGPGAWQGGRRKFRRQRPRLSHKGPMPF
Number of amino acids: 161
Molecular weight: 17898.88
Theoretical pl: 4.53
Solubility: 0.599
CDP2.2-Apelin (CDP2.2/IDP2 in bold, linker
underlined, Apelin in italics)
(SEQ ID NO: 51)
MHHAHNPGIDAEDANVQQFNAQKYVLQDGDIMWQVEGEFQLEPPLDTEEGRAAADEDDEDFVDE
DDDGSGSGSGSNLRLCVQALLLLWLSLTAVCGVPLMLPPDGTGLEEGSMRYLVKPRTSRTGPGA
WQGGRRKFRRQRPRLSHKGPMPF
Number of amino acids: 151
Molecular weight: 16702.59
Theoretical pl: 4.73
Solubility: 0.648
CDP2.3-Apelin (CDP2.3/IDP3 sequence in bold,
linker underlined, Apelin in italics)
(SEQ ID NO: 52)
MHHAH [symbol] [symbol] [symbol] [symbol] NLRLCVQALL
LLWLSLTAVCGVPLMLPPDGTGLEEGSMRYLVKPRTSRTGPGAWQGGRRKFRRQRPRLSHKGPM
PF
Number of amino acids: 130
Molecular weight: 14417.23
Theoretical pl: 5.73
Solubility: 0.470
TRXtr-Notum (TRXtr in bold, linker
underlined, Notum in italics)
(SEQ ID NO: 53)
HMHHHHHHGKLTVAKLNIDQNPGTAPKYGIRGIPTLLLFKNGEVAATKVGALSKGQLKEFLDAN
LAGSGSGSGSSEGRKTWRRRGQQPPQPPPPPPLPQRAEVEPGAGQPVESFPLDFTAVEGNMDSF
MAQVKSLAQSLYPCSAQQLNEDLRLHLLLNTSVTCNDGSPAGYYLKESKGSRRWLLFLEGGWYC
FNRENCDSRYSTMRRLMSSKDWPHTRTGTGILSSQPEENPHWWNANMVFIPYCSSDVWSGASPK
SDKNEYAFMGSLIIQEVVRELLGKGLSGAKVLLLAGSAAGGTGVLLNVDRVAELLEELGYPSIQ
VRGLADSGWFLDNKQYRRSDCIDTINCAPTDAIRRGIRYWSGMVPERCQRQFKEGEEWNCFFGY
KVYPTLRCPVFVVQWLFDEAQLTVDNVHLTGQPVQEGQWLYIQNLGRELRGTLKDVQASFAPAC
LSHEIIIRSYWTDVQVKGTSLPRALHCWDRSFHDSHKASKTPMKGCPFHLVDSCPWPHCNPSCP
TIRDQFTGQEMNVAQFLMHMGFDVQTVAQQQGMEPSKLLGMLSNGN
Number of amino acids: 558
Molecular weight: 62281.72Da (62 kDa)
Theoretical pl: 7.90
CDP - mNotum (CDP in bold, linker
underlined, mNotum in italics)
(SEQ ID NO: 54)
HMHHHHHHDNNSLSQEVQNGSNHLENNQSQSNGGGSDSALSLSSKTAALAAATTVNDGSDGATS
SAVGGSGSGSGSSEGRKTWRRRGQQPPQPPPPPPLPQRAEVEPGAGQPVESFPLDFTAVEGNMD
SFMAQVKSLAQSLYPCSAQQLNEDLRLHLLLNTSVTCNDGSPAGYYLKESKGSRRWLLFLEGGW
YCFNRENCDSRYSTMRRLMSSKDWPHTRTGTGILSSQPEENPHWWNANMVFIPYCSSDVWSGAS
PKSDKNEYAFMGSLIIQEVVRELLGKGLSGAKVLLLAGSAAGGTGVLLNVDRVAELLEELGYPS
IQVRGLADSGWFLDNKQYRRSDCIDTINCAPTDAIRRGIRYWSGMVPERCQRQFKEGEEWNCFF
GYKVYPTLRCPVFVVQWLFDEAQLTVDNVHLTGQPVQEGQWLYIQNLGRELRGTLKDVQASFAP
ACLSHEIIIRSYWTDVQVKGTSLPRALHCWDRSFHDSHKASKTPMKGCPFHLVDSCPWPHCNPS
CPTIRDQFTGQEMNVAQFLMHMGFDVQTVAQQQGMEPSKLLGMLSNGN
Number of amino acids: 560
Molecular weight: 62011.51 Da (62kDa)
Theoretical pl: 6.27
CDP2.1-mNotum (CDP2.1/IPD1 sequence in
bold, linker underlined, mNotum in italics)
(SEQ ID NO: 55)
HMHHAHTMYATLEEAIDAAREEFLADNPGIDAEDANVQQFNAQKYVLQDGDIMWQVEFFADEGE
EGEDDEDFVDEDDDGSGSGSGSSEGRKTWRRRGQQPPQPPPPPPLPQRAEVEPGAGQPVESFPL
DFTAVEGNMDSFMAQVKSLAQSLYPCSAQQLNEDLRLHLLLNTSVTCNDGSPAGYYLKESKGSR
RWLLFLEGGWYCFNRENCDSRYSTMRRLMSSKDWPHTRTGTGILSSQPEENPHWWNANMVFIPY
CSSDVWSGASPKSDKNEYAFMGSLIIQEVVRELLGKGLSGAKVLLLAGSAAGGTGVLLNVDRVA
ELLEELGYPSIQVRGLADSGWFLDNKQYRRSDCIDTINCAPTDAIRRGIRYWSGMVPERCQRQF
KEGEEWNCFFGYKVYPTLRCPVFVVQWLFDEAQLTVDNVHLTGQPVQEGQWLYIQNLGRELRGT
LKDVQASFAPACLSHEIIIRSYWTDVQVKGTSLPRALHCWDRSFHDSHKASKTPMKGCPFHLVD
SCPWPHCNPSCPTIRDQFTGQEMNVAQFLMHMGFDVQTVAQQQGMEPSKLLGMLSNGN
Number of amino acids: 570
Molecular weight: 64075.80 Da (64 kDa)
Theoretical pl: 5.00
CDP2.2- mNotum (CDP2.2/IPD2 sequence in
bold, linker underlined, mNotum in italics)
(SEQ ID NO: 56)
HMHHAHNPGIDAEDANVQQFNAQKYVLQDGDIMWQVEGEFQLEPPLDTEEGRAAADEDDEDFVD
EDDDGSGSGSGSSEGRKTWRRRGQQPPQPPPPPPLPQRAEVEPGAGQPVESFPLDFTAVEGNMD
SFMAQVKSLAQSLYPCSAQQLNEDLRLHLLLNTSVTCNDGSPAGYYLKESKGSRRWLLFLEGGW
YCFNRENCDSRYSTMRRLMSSKDWPHTRTGTGILSSQPEENPHWWNANMVFIPYCSSDVWSGAS
PKSDKNEYAFMGSLIIQEVVRELLGKGLSGAKVLLLAGSAAGGTGVLLNVDRVAELLEELGYPS
IQVRGLADSGWFLDNKQYRRSDCIDTINCAPTDAIRRGIRYWSGMVPERCQRQFKEGEEWNCFF
GYKVYPTLRCPVFVVQWLFDEAQLTVDNVHLTGQPVQEGQWLYIQNLGRELRGTLKDVQASFAP
ACLSHEIIIRSYWTDVQVKGTSLPRALHCWDRSFHDSHKASKTPMKGCPFHLVDSCPWPHCNPS
CPTIRDQFTGQEMNVAQFLMHMGFDVQTVAQQQGMEPSKLLGMLSNGN
Number of amino acids: 560
Molecular weight: 62879.51 Da (63 kDa)
Theoretical pl: 5.14
CDP2.3- mNotum (CPD2.3/IPD3 sequence in
bold, linker underlined, mNotum in italics)
(SEQ ID NO: 57)
HMHHAHNPGIDAEDANVQQFNAQKYVLQDGDIMWQVDDEDFVDEDDDGSGSGSGSSEGRKTWRR
RGQQPPQPPPPPPLPQRAEVEPGAGQPVESFPLDFTAVEGNMDSFMAQVKSLAQSLYPCSAQQL
NEDLRLHLLLNTSVTCNDGSPAGYYLKESKGSRRWLLFLEGGWYCFNRENCDSRYSTMRRLMSS
KDWPHTRTGTGILSSQPEENPHWWNANMVFIPYCSSDVWSGASPKSDKNEYAFMGSLIIQEVVR
ELLGKGLSGAKVLLLAGSAAGGTGVLLNVDRVAELLEELGYPSIQVRGLADSGWFLDNKQYRRS
DCIDTINCAPTDAIRRGIRYWSGMVPERCQRQFKEGEEWNCFFGYKVYPTLRCPVFVVQWLFDE
AQLTVDNVHLTGQPVQEGQWLYIQNLGRELRGTLKDVQASFAPACLSHEIIIRSYWTDVQVKGT
SLPRALHCWDRSFHDSHKASKTPMKGCPFHLVDSCPWPHCNPSCPTIRDQFTGQEMNVAQFLMH
MGFDVQTVAQQQGMEPSKLLGMLSNGN
Number of amino acids: 539
Molecular weight: 60594.15 Da (60.5 kDa)
Theoretical pl: 5.49
TRXtr-Timp1 (TRXtr in bold, linker
underlined, Timp1 in italics)
(SEQ ID NO: 58)
HMHHHHHHGKLTVAKLNIDQNPGTAPKYGIRGIPTLLLFKNGEVAATKVGALSKGQLKEFLDAN
LAGSGSGSGSCSCAPPHPQTAFCNSDLVIRAKFMGSPEINETTLYQRYKIKMTKMLKGFKAVGN
AADIRYAYTPVMESLCGYAHKSQNRSEEFLITGRLRNGNLHISACSFLVPWRTLSPAQQRAFSK
TYSAGCGVCTVFPCLSIPCKLESDTHCLWTDQVLVGSEDYQSRHFACLPRNPGLCTWRSLGAR
Number of amino acids: 255
Molecular weight: 27955.14 Da (27 kDa)
Theoretical pl: 9.30
CDP-Timp1 (CDP in bold, linker
underlined, Timp1 in italics)
(SEQ ID NO: 59)
HMHHHHHHDNNSLSQEVQNGSNHLENNQSQSNGGGSDSALSLSSKTAALAAATTVNDGSDGATS
SAVGGSGSGSGSCSCAPPHPQTAFCNSDLVIRAKFMGSPEINETTLYQRYKIKMTKMLKGFKAV
GNAADIRYAYTPVMESLCGYAHKSQNRSEEFLITGRLRNGNLHISACSFLVPWRTLSPAQQRAF
SKTYSAGCGVCTVFPCLSIPCKLESDTHCLWTDQVLVGSEDYQSRHFACLPRNPGLCTWRSLGA
R
Number of amino acids: 257
Molecular weight: 27684.93 Da (28 kDa)
Theoretical pl: 8.46
CDP2.1-Timp1 (CDP2.1 in bold, linker
underlined, Timp1 in italics)
(SEQ ID NO: 60)
HMHHAHTMYATLEEAIDAAREEFLADNPGIDAEDANVQQFNAQKYVLQDGDIMWQVEFFADEGE
EGEDDEDFVDEDDDGSGSGSGSCSCAPPHPQTAFCNSDLVIRAKFMGSPEINETTLYQRYKIKM
TKMLKGFKAVGNAADIRYAYTPVMESLCGYAHKSQNRSEEFLITGRLRNGNLHISACSFLVPWR
TLSPAQQRAFSKTYSAGCGVCTVFPCLSIPCKLESDTHCLWTDQVLVGSEDYQSRHFACLPRNP
GLCTWRSLGAR
Number of amino acids: 267
Molecular weight: 29749.22 Da (30 kDa)
Theoretical pl: 4.95
CDP2.2-Timp1 (CDP2.2 in bold, linker
underlined, Timp1 in italics)
(SEQ ID NO: 61)
HMHHAHNPGIDAEDANVQQFNAQKYVLQDGDIMWQVEGEFQLEPPLDTEEGRAAADEDDEDFVD
EDDDGSGSGSGSCSCAPPHPQTAFCNSDLVIRAKFMGSPEINETTLYQRYKIKMTKMLKGFKAV
GNAADIRYAYTPVMESLCGYAHKSQNRSEEFLITGRLRNGNLHISACSFLVPWRTLSPAQQRAF
SKTYSAGCGVCTVFPCLSIPCKLESDTHCLWTDQVLVGSEDYQSRHFACLPRNPGLCTWRSLGA
R
Number of amino acids: 257
Molecular weight: 28552.93 Da (28.5 kDa)
Theoretical pl: 5.23
CDP2.3-Timp1 (CDP2.3 in bold, linker
underlined, Timp1 in italics)
(SEQ ID NO: 62)
HMHHAHNPGIDAEDANVQQFNAQKYVLQDGDIMWQVDDEDFVDEDDDGSGSGSGSCSCAPPHPQ
TAFCNSDLVIRAKFMGSPEINETTLYQRYKIKMTKMLKGFKAVGNAADIRYAYTPVMESLCGYA
HKSQNRSEEFLITGRLRNGNLHISACSFLVPWRTLSPAQQRAFSKTYSAGCGVCTVFPCLSIPC
KLESDTHCLWTDQVLVGSEDYQSRHFACLPRNPGLCTWRSLGAR
Number of amino acids: 236/
Molecular weight: 26267.57 Da (26 kDa)
Theoretical pl: 6.13
CDP-complete (HisTag-CDP-GS-linker-RBM-GS-
linker-SD1-GS-linker-SD2-GS-linker-S1/S2 -
GS-linker-FP-GS-linker-HR1 protein sequence
(357 aa) - CDP in bold, linkers underlined
and SARS-COV-2 sequences italicised)
(SEQ ID NO: 63)
MHHHHHHDNNSLSQEVQNGSNHLENNQSQSNGGGSDSALSLSSKTAALAAATTVNDGSDGATSS
AVGGSGSGSGSSNNLDSKVGGNYNYLYRLFRKSNLKPFERDISTEIYQAGSTPCNGVEGFNCYF
PLQSYGFQPTNGVGYQGSGSGSPKKSTNLVKNKCVNFNFNGLTGTGVLTESNKKFLPFGSGSGS
TEVPVAIHADQLTPTWRVYSTGSNVFQTRGSGSGSYQTQTNSPRRARSVASQSIIAYTGSGSGS
KQIYKTPPIKDFGGFNFSQILPDPSKPSKRSFIEDLLFNKVTLADAGFIKQYGDCLGDIAARDG
SGSGSTQNVLYENQKLIANQFNSAIGKIQDSLSSTAS
Theoretical pl/Mw: 8.89/37458.79
CDP-binding (HisTag-CDP-GS-linker-RBM-GS-
linker-SD1-GS-linker-SD2 protein sequence -
CDP in bold, linkers underlined and SARS-
COV-2 sequences italicised) (221 aa)
(SEQ ID NO: 64)
MHHHHHHDNNSLSQEVQNGSNHLENNQSQSNGGGSDSALSLSSKTAALAAATTVNDGSDGATSS
AVGGSGSGSGSSNNLDSKVGGNYNYLYRLFRKSNLKPFERDISTEIYQAGSTPCNGVEGFNCYF
PLQSYGFQPTNGVGYQGSGSGSPKKSTNLVKNKCVNFNFNGLTGTGVLTESNKKFLPFGSGSGS
TEVPVAIHADQLTPTWRVYSTGSNVFQTR
Theoretical pl/Mw: 7.91/23095.91
CDP-fusion (HisTag-CDP-GS-linker-S1/S2-GS-
linker-FP-GS-linker-HR1 protein sequence -
CDP in bold, linkers underlined, SARS-COV-2
sequences italicised) (205 aa)
(SEQ ID NO: 65)
MHHHHHHDNNSLSQEVQNGSNHLENNQSQSNGGGSDSALSLSSKTAALAAATTVNDGSDGATSS
AVGGSGSGSGSYQTQTNSPRRARSVASQSIIAYTGSGSGSKQIYKTPPIKDFGGFNFSQILPDP
SKPSKRSFIEDLLFNKVTLADAGFIKQYGDCLGDIAARDGSGSGSTQNVLYENQKLIANQFNSA
IGKIQDSLSSTAS
Theoretical pl/Mw: 6.71/21124.75
DNA sequence for “CDP-complete” (HisTag-CDP-GS-
linker-RBM-GS-linker-SD1-GS-linker-SD2-GS-
linker-S1/S2-GS-linker-FP-GS-linker-HR1)
(1089 nt)
SEQ ID NO: 66)
CATATGCACCATCACCATCACCATGATAACAACTCACTGTCGCAGGAAGTTCAAAATGGTTCTA
ACCACTTAGAAAACAACCAGAGCCAGAGTAATGGCGGCGGTTCTGATTCGGCTCTGTCCCTCAG
TTCTAAAACAGCGGCTCTGGCCGCTGCCACGACGGTTAATGATGGTTCTGATGGAGCAACCAGT
TCTGCTGTCGGTGGTTCTGGTTCTGGTAGCGGATCCAGCAACAACCTGGATAGCAAAGTGGGCG
GCAACTATAACTATCTGTATCGCCTGTTTCGCAAAAGCAACCTGAAACCGTTTGAACGCGATAT
TAGCACCGAAATTTATCAGGCGGGCAGCACCCCGTGCAACGGCGTGGAAGGCTTTAACTGCTAT
TTTCCGCTGCAGAGCTATGGCTTTCAGCCGACCAACGGCGTGGGCTATCAGGGTTCTGGTTCTG
GTAGCCCGAAAAAAAGCACCAACCTGGTGAAAAACAAATGCGTGAACTTTAACTTTAACGGCCT
GACCGGCACCGGCGTGCTGACCGAAAGCAACAAAAAATTTCTGCCGTTTGGTTCTGGTTCTGGT
AGCACCGAAGTGCCGGTGGCGATTCATGCGGATCAGCTGACCCCGACCTGGCGCGTGTATAGCA
CCGGCAGCAACGTGTTTCAGACCCGCGGTTCTGGTTCTGGTAGCTATCAGACCCAGACCAACAG
CCCGCGCCGCGCGCGCAGCGTGGCGAGCCAGAGCATTATTGCGTATACCGGTTCTGGTTCTGGT
AGCAAACAGATTTATAAAACCCCGCCGATTAAAGATTTTGGCGGCTTTAACTTTAGCCAGATTC
TGCCGGATCCGAGCAAACCGAGCAAACGCAGCTTTATTGAAGATCTGCTGTTTAACAAAGTGAC
CCTGGCGGATGCGGGCTTTATTAAACAGTATGGCGATTGCCTGGGCGATATTGCGGCGCGCGAT
GGTTCTGGTTCTGGTAGCACCCAGAACGTGCTGTATGAAAACCAGAAACTGATTGCGAACCAGT
TTAACAGCGCGATTGGCAAAATTCAGGATAGCCTGAGCAGCACCGCGAGCTAGTGATAACTCGA
G
Additional information - (restriction sites in italics,
his-tag in bold, linkers underlined, 3x stop underlined
and bold): pET21a (+) - Nde1 - HisTag (18 nt) - CDP
(180 nt) -GS-linker (18nt)- BamHI-RBM (207 nt) -GS-linker
(18nt)- SD1(108 nt) -GS-linker (18nt)- SD2 (87nt) -GS-
linker (18nt)- S1/S2 (69nt) -GS-linker (18nt)- FP (189nt)
-GS-linker (18nt)- HR1 (96nt) - 3x stop Xho1 - pET21a
DNA sequence for “CDP-binding” (HisTag-CDP-GS-linker-
RBM-GS-linker-SD1-GS-linker-SD2) (681 nt)
(SEQ ID NO: 67)
CATATGCACCATCACCATCACCATGATAACAACTCACTGTCGCAGGAAGTTCAAAATGGTTCTA
ACCACTTAGAAAACAACCAGAGCCAGAGTAATGGCGGCGGTTCTGATTCGGCTCTGTCCCTCAG
TTCTAAAACAGCGGCTCTGGCCGCTGCCACGACGGTTAATGATGGTTCTGATGGAGCAACCAGT
TCTGCTGTCGGTGGTTCTGGTTCTGGTAGCGGATCCAGCAACAACCTGGATAGCAAAGTGGGCG
GCAACTATAACTATCTGTATCGCCTGTTTCGCAAAAGCAACCTGAAACCGTTTGAACGCGATAT
TAGCACCGAAATTTATCAGGCGGGCAGCACCCCGTGCAACGGCGTGGAAGGCTTTAACTGCTAT
TTTCCGCTGCAGAGCTATGGCTTTCAGCCGACCAACGGCGTGGGCTATCAGGGTTCTGGTTCTG
GTAGCCCGAAAAAAAGCACCAACCTGGTGAAAAACAAATGCGTGAACTTTAACTTTAACGGCCT
GACCGGCACCGGCGTGCTGACCGAAAGCAACAAAAAATTTCTGCCGTTTGGTTCTGGTTCTGGT
AGCACCGAAGTGCCGGTGGCGATTCATGCGGATCAGCTGACCCCGACCTGGCGCGTGTATAGCA
CCGGCAGCAACGTGTTTCAGACCCGCTAGTGATAACTCGAG
Additional information - (restriction sites in italics,
his-tag in bold, linkers underlined, 3x stop underlined
and bold): pET21a (+) - Nde1 - HisTag (18 nt) - CDP (180 nt)
-GS-linker (18nt)- BamHI-RBM (207 nt) -GS-linker (18nt)-
SD1(108 nt) -GS-linker (18nt)- SD2 (87nt) - 3x stop Xho1 -
pET21a
DNA sequence for “CDP-fusion” (HisTag-CDP-GS-linker-S1/S2-
GS-linker-FP-GS-linker-HR1)
(SEQ ID NO: 68)
CATATGCACCATCACCATCACCATGATAACAACTCACTGTCGCAGGAAGTTCAAAATGGTTCTA
ACCACTTAGAAAACAACCAGAGCCAGAGTAATGGCGGCGGTTCTGATTCGGCTCTGTCCCTCAG
TTCTAAAACAGCGGCTCTGGCCGCTGCCACGACGGTTAATGATGGTTCTGATGGAGCAACCAGT
TCTGCTGTCGGTGGTTCTGGTTCTGGTAGCGGATCCTATCAGACCCAGACCAACAGCCCGCGCC
GCGCGCGCAGCGTGGCGAGCCAGAGCATTATTGCGTATACCGGTTCTGGTTCTGGTAGCAAACA
GATTTATAAAACCCCGCCGATTAAAGATTTTGGCGGCTTTAACTTTAGCCAGATTCTGCCGGAT
CCGAGCAAACCGAGCAAACGCAGCTTTATTGAAGATCTGCTGTTTAACAAAGTGACCCTGGCGG
ATGCGGGCTTTATTAAACAGTATGGCGATTGCCTGGGCGATATTGCGGCGCGCGATGGTTCTGG
TTCTGGTAGCACCCAGAACGTGCTGTATGAAAACCAGAAACTGATTGCGAACCAGTTTAACAGC
GCGATTGGCAAAATTCAGGATAGCCTGAGCAGCACCGCGAGCTAGTGATAACTCGAG
Additional information - (restriction sites in italics,
his-tag in bold, linkers underlined, 3x stop underlined
and bold): pET21a (+) - Nde1 - HisTag (18 nt) - CDP
(180 nt) -GS-linker (18nt)- BamHI-S1/S2 (69nt) -GS-linker
(18nt)- FP (189nt) -GS-linker (18nt)- HR1 (96nt) -
3x stop Xho1 - pET21a
RBM of SARS-COV-2
(SEQ ID NO: 69)
SNNLDSKVGGNYNYLYRLFRKSNLKPFERDISTEIYQAGSTPCNGVEGFNCYFPLQSYGFQPTN
GVGYQ
SD1 of SARS-COV-2
(SEQ ID NO: 70)
PKKSTNLVKNKCVNFNFNGLTGTGVLTESNKKFLPF
SD2 of SARS-COV-2
(SEQ ID NO: 71)
TEVPVAIHADQLTPTWRVYSTGSNVFQTR
S1/S2 of SARS-COV-2
(SEQ ID NO: 72)
YQTQTNSPRRARSVASQSIIAYT
FP of SARS-COV-2
(SEQ ID NO: 73)
KQIYKTPPIKDFGGFNFSQILPDPSKPSKRSFIEDLLFNKVTLADAGFIKQYGDCLGDIAARD
HR1 of SARS-COV-2
(SEQ ID NO: 74)
TQNVLYENQKLIANQFNSAIGKIQDSLSSTAS

1. A fusion polypeptide for use in vaccinating or treating a mammalian subject, the fusion polypeptide comprising:
(i) a non-mammalian peptide sequence, wherein the peptide sequence is a sequence that is associated with a disease or disorder against which the mammalian subject is being vaccinated or treated; and
(ii) an immune stimulating peptide sequence having a ratio of bulky hydrophilic or charged amino acids to total amino acids of less than or equal to 0.55.

2. The fusion polypeptide for use according to claim 1, wherein the fusion polypeptide has an isoelectric point (pI) of less than or equal to 6.

3. The fusion polypeptide for use according to claim 1 or claim 2, wherein:
a) the non-mammalian peptide sequence is a viral peptide and the disease or disorder is a viral infection; or
b) the non-mammalian peptide sequence is a mycobacterial or a bacterial peptide and the disease or disorder is a mycobacterial or a bacterial infection; or
c) the non-mammalian peptide sequence is a yeast peptide and the disease or disorder is a yeast infection;
d) the non-mammalian peptide sequence is a parasite peptide and the disease or disorder is a parasite infection.

4. The fusion polypeptide for use according to any one of claims 1 to 3, wherein the non-mammalian peptide sequence is a SARS-CoV-2 peptide and the disease or disorder is a SARS-CoV-2 infection.

5. The fusion polypeptide for use according to claim 4, wherein the SARS-CoV-2 peptide is a SARS-CoV-2 receptor binding domain (RBD) peptide.

6. The fusion polypeptide for use according to claim 5, wherein the RBD peptide comprises the amino acid sequence: NITNLCPFGEVFNATRFASVYAWNRKRISNCVADYSVLYNSASFSTFKCYGVSPTKLNDLCFTN VYADSFVIRGDEVRQIAPGQTGKIADYNYKLPDDFTGCVIAWNSNNLDSKVGGNYNYLYRLFRK SNLKPFERDISTEIYQAGSTPCNGVEGFNCYFPLQSYGFQPTNGVGYQPYRVVVLSFELLHAP ATV (SEQ ID NO:1), or a conservative amino acid variant thereof.

7. The fusion polypeptide for use according to any one of claims 4 to 6, wherein the SARS-CoV-2 peptide is a S protein peptide, optionally wherein:
(a) the S protein peptide is a S1 subdomain peptide, further optionally wherein the S1 subdomain peptide is a RBD peptide (such as a receptor binding motif (RBM) peptide), a SD1 peptide or a SD2 peptide, or a combination thereof;
(b) the S protein peptide is a S2 subdomain peptide, further optionally wherein the S2 subdomain peptide is a S1/S2 peptide, a FP+S2′ peptide, or a HR1 peptide, or a combination thereof.

8. The fusion polypeptide for use according to claim 7, wherein the SARS-CoV-2 peptide comprises the amino acid sequence:
(a) of SEQ ID NO:39, or a conservative amino acid variant thereof;
(b) of SEQ ID NO:40, or a conservative amino acid variant thereof; or
(c) of SEQ ID NO:41, or a conservative amino acid variant thereof.

9. The fusion polypeptide for use according to any one of claims 1 to 3, wherein the non-mammalian peptide sequence is an influenza virus peptide sequence and the disease or disorder is influenza.

10. The fusion polypeptide for use according to claim 9, wherein the influenza virus peptide sequence is a H1 influenza virus peptide, a N1 influenza virus peptide, or a H1/N1 influenza virus peptide.

11. The fusion polypeptide for use according to claim 10, wherein the influenza virus peptide comprises the amino acid sequence:
(a) of SEQ ID NO:42, or a conservative amino acid variant thereof;
(b) of SEQ ID NO:43, or a conservative amino acid variant thereof; or
(c) of SEQ ID NO:44, or a conservative amino acid variant thereof.

12. The fusion polypeptide for use according to any one of the preceding claims, wherein the immune stimulating peptide sequence is at least 40 amino acids long and is no more than 150 amino acids long.

13. The fusion polypeptide for use according to any one of the preceding claims, wherein the immune stimulating peptide sequence has an isoelectric point (pI) of less than or equal to 4.

14. The fusion polypeptide for use according to any one of the preceding claims, wherein the immune stimulating peptide sequence comprises the amino acid sequence DDEDFVDEDDD (SEQ ID NO:4) of the RRAB protein or a conservative amino acid variant thereof.

15. The fusion polypeptide for use according to any one of the preceding claims, wherein the immune stimulating peptide sequence comprises at least one predicted T cell epitope.

16. The fusion polypeptide for use according to claim 15, wherein the predicted T cell epitopes are selected from: a MSYB protein T cell epitope and/or a RRAB protein T cell epitope.

17. The fusion polypeptide for use according to claim 16, wherein the MSYB protein T cell epitope is comprised within the amino sequence: NPGIDAEDANVQQFNAQKYVLQDGDIMWQV (SEQ ID NO:2), and/or EGEFQLEPPLDTEEGRAAADE (SEQ ID NO:3), or a conservative amino acid variant thereof.

18. The fusion polypeptide for use according to any one of the preceding claims, wherein the immune stimulating peptide sequence comprises the amino acid sequence:
a) DNNSLSQEVQNGSNHLENNQSQSNGGGSDSALSLSSKTAALAAATTVNDGSDGA TSSAVG (SEQ ID NO:5), or a conservative amino acid variant thereof; or
b) HHAHTMYATLEEAIDAAREEFLADNPGIDAEDANVQQFNAQKYVLQDGDIMWQVE FFADEGEEGEDDEDFVDEDDD (SEQ ID NO:6), or a conservative amino acid variant thereof; or
c) HHAHNPGIDAEDANVQQFNAQKYVLQDGDIMWQVEGEFQLEPPLDTEEGRAAAD EDDEDFVDEDDD (SEQ ID NO:7), or a conservative amino acid variant thereof; or
d) HHAHNPGIDAEDANVQQFNAQKYVLQDGDIMWQVDDEDFVDEDDD (SEQ ID NO:8), or a conservative amino acid variant thereof.

19. A fusion polypeptide comprising:
(i) a non-mammalian peptide sequence, wherein the peptide sequence is a sequence that is associated with a disease or disorder against which the mammalian subject is being vaccinated or treated; and
(ii) an immune stimulating peptide sequence having a ratio of bulky hydrophilic or charged amino acids to total amino acids of less than or equal to 0.55;
wherein the non-mammalian peptide sequence is linked to the immune stimulating peptide sequence and wherein the immune stimulating peptide sequence comprises the amino acid sequence:
a) HHAHTMYATLEEAIDAAREEFLADNPGIDAEDANVQQFNAQKYVLQDGDIMWQVE FFADEGEEGEDDEDFVDEDDD (SEQ ID NO:6), or a conservative amino acid variant thereof; or
b) HHAHNPGIDAEDANVQQFNAQKYVLQDGDIMWQVEGEFQLEPPLDTEEGRAAAD EDDEDFVDEDDD (SEQ ID NO:7), or a conservative amino acid variant thereof; or
c) HHAHNPGIDAEDANVQQFNAQKYVLQDGDIMWQVDDEDFVDEDDD (SEQ ID NO:8), or a conservative amino acid variant thereof; or
d) DNNSLSQEVQNGSNHLENNQSQSNGGGSDSALSLSSKTAALAAATTVNDGSDGA TSSAVG (SEQ ID NO: 5), or a conservative amino acid variant thereof.

20. A fusion polypeptide comprising:
(i) a mammalian peptide sequence, wherein the peptide sequence is a sequence that is associated with a disease or disorder against which the mammalian subject is being vaccinated or treated; and
(ii) an immune stimulating peptide sequence having a ratio of bulky hydrophilic or charged amino acids to total amino acids of less than or equal to 0.55;
wherein the mammalian peptide sequence is linked to the immune stimulating peptide sequence and wherein the immune stimulating peptide sequence comprises the amino acid sequence:
d) HHAHTMYATLEEAIDAAREEFLADNPGIDAEDANVQQFNAQKYVLQDGDIMWQVE FFADEGEEGEDDEDFVDEDDD (SEQ ID NO:6), or a conservative amino acid variant thereof; or
e) HHAHNPGIDAEDANVQQFNAQKYVLQDGDIMWQVEGEFQLEPPLDTEEGRAAAD EDDEDFVDEDDD (SEQ ID NO:7), or a conservative amino acid variant thereof; or
f) HHAHNPGIDAEDANVQQFNAQKYVLQDGDIMWQVDDEDFVDEDDD (SEQ ID NO:8), or a conservative amino acid variant thereof.

21. The fusion polypeptide according to claim 19 or 20, wherein the fusion polypeptide has an isoelectric point (pI) of less than or equal to 6.

22. The fusion polypeptide according to claim 19 or 21, wherein:
a) the non-mammalian peptide sequence is a viral peptide and the disease or disorder is a viral infection; or
b) the non-mammalian peptide sequence is a mycobacterial or a bacterial peptide and the disease or disorder is a mycobacterial or a bacterial infection; or
c) the non-mammalian peptide sequence is a yeast peptide and the disease or disorder is a yeast infection;
d) the non-mammalian peptide sequence is a parasite peptide and the disease or disorder is a parasite infection.

23. The fusion polypeptide according to claim 20, wherein the mammalian peptide sequence is associated with tumor angiogenesis.

24. The fusion polypeptide according to claim 20, wherein the mammalian peptide sequence is vimentin (Vim), apelin, notum or timp1.

25. The fusion polypeptide according to any one of claims 19 and 21 to 22, wherein the non-mammalian peptide sequence is a SARS-CoV-2 peptide.

26. The fusion polypeptide according to claim 25, wherein the SARS-CoV-2 peptide is a SARS-CoV-2 receptor binding domain (RBD) peptide.

27. The fusion polypeptide according to claim 26, wherein the RBD peptide comprises the amino acid sequence: NITNLCPFGEVFNATRFASVYAWNRKRISNCVADYSVLYNSASFSTFKCYGVSPTKLNDLCFTN VYADSFVIRGDEVRQIAPGQTGKIADYNYKLPDDFTGCVIAWNSNNLDSKVGGNYNYLYRLFRK SNLKPFERDISTEIYQAGSTPCNGVEGFNCYFPLQSYGFQPTNGVGYQPYRVVVLSFELLHAP ATV (SEQ ID NO:1), or a conservative amino acid variant thereof.

28. The fusion polypeptide according to any one of claims 25 to 27, wherein the SARS-CoV-2 peptide is a S protein peptide, optionally wherein:
(a) the S protein peptide is a S1 subdomain peptide, further optionally wherein the S1 subdomain peptide is a RBD peptide (such as a receptor binding motif (RBM) peptide), a SD1 peptide or a SD2 peptide, or a combination thereof;
(b) the S protein peptide is a S2 subdomain peptide, further optionally wherein the S2 subdomain peptide is a S1/S2 peptide, a FP+S2′ peptide, or a HR1 peptide, or a combination thereof.

29. The fusion polypeptide according to claim 28, wherein the SARS-CoV-2 peptide comprises the amino acid sequence:
(a) of SEQ ID NO:39, or a conservative amino acid variant thereof;
(b) of SEQ ID NO:40, or a conservative amino acid variant thereof; or
(c) of SEQ ID NO:41, or a conservative amino acid variant thereof.

30. The fusion polypeptide according to any one of claims 19 and 21 to 22, wherein the non-mammalian peptide sequence is an influenza virus peptide sequence and the disease or disorder is influenza.

31. The fusion polypeptide according to claim 30, wherein the influenza virus peptide sequence is a H1 influenza virus peptide, a N1 influenza virus peptide, or a H1/N1 influenza virus peptide.

32. The fusion polypeptide according to claim 31, wherein the influenza virus peptide comprises the amino acid sequence:
(a) of SEQ ID NO:42, or a conservative amino acid variant thereof;
(b) of SEQ ID NO:43, or a conservative amino acid variant thereof; or
(c) of SEQ ID NO:44, or a conservative amino acid variant thereof.

33. The fusion polypeptide according to any one of claims 19 to 32, wherein the immune stimulating peptide sequence is at least 40 amino acids long and is no more than 150 amino acids long.

34. The fusion polypeptide according to any one of claims 19 to 33, wherein the immune stimulating peptide sequence has an isoelectric point (pI) of less than or equal to 4.

35. A nucleic acid encoding a fusion polypeptide according to any one of claims 19 to 34, optionally wherein the nucleic acid is an expression vector.

36. A pharmaceutical composition comprising a fusion polypeptide according to any one of claims 19 to 34, or a nucleic acid according to claim 35, wherein the composition further comprises a pharmaceutically acceptable carrier, adjuvant, excipient or diluent.

37. A fusion polypeptide according to any one of claims 19 to 34, or a composition according to claim 36, for use as a medicament.

38. Use of a fusion polypeptide according to any one of claims 19 to 34, or a composition according to claim 36, for vaccination or treatment of a subject.

39. A method for vaccinating or treating a subject, the method comprising administering a fusion polypeptide according to any one of claims 19 to 34, or a composition according to claim 36, to the subject.


SEQUENCE LISTING (75 sequences)

SEQ ID NO: 1 - protein, 194 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 2 - protein, 30 aa, Escherichia coli
NPGIDAEDANVQQFNAQKYVLQDGDIMWQV

SEQ ID NO: 3 - protein, 21 aa, Escherichia coli
EGEFQLEPPLDTEEGRAAADE

SEQ ID NO: 4 - protein, 11 aa, Escherichia coli
DDEDFVDEDDD

SEQ ID NO: 5 - protein, 60 aa, synthetic construct
  REGION 1..60: note = Immune stimulating peptide sequence [original CDP]
DNNSLSQEVQNGSNHLENNQSQSNGGGSDSALSLSSKTAALAAATTVNDGSDGATSSAVG

SEQ ID NO: 6 - protein, 76 aa, synthetic construct
  REGION 1..76: note = Immune stimulating peptide sequence [IDP1]
HHAHTMYATLEEAIDAAREEFLADNPGIDAEDANVQQFNAQKYVLQDGDIMWQVEFFADEGEEGEDDEDFVDEDDD

SEQ ID NO: 7 - protein, 66 aa, synthetic construct
  REGION 1..66: note = Immune stimulating peptide sequence [IDP2]
HHAHNPGIDAEDANVQQFNAQKYVLQDGDIMWQVEGEFQLEPPLDTEEGRAAADEDDEDFVDEDDD

SEQ ID NO: 8 - protein, 45 aa, synthetic construct
  REGION 1..45: note = Immune stimulating peptide sequence [IDP3]
HHAHNPGIDAEDANVQQFNAQKYVLQDGDIMWQVDDEDFVDEDDD

SEQ ID NO: 9 - protein, 108 aa, Escherichia coli
SDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 10 - protein, 58 aa, Escherichia coli
GKLTVAKLNIDQNPGTAPKYGIRGIPTLLLFKNGEVAATKVGALSKGQLKEFLDANLA

SEQ ID NO: 11 - protein, 131 aa, Escherichia coli
AATTVNGGTVHFKGEVVNAACAVDAGSVDQTVQLGQVRTASLAQEGATSSAVGFNIQLNDCDTNVASKAAVAFLGTAIDAGHTNVLALQSSAAGSATNVGVQILDRTGAALTLDGATFSSETTLNNGTNTI

SEQ ID NO: 12 - protein, 131 aa, synthetic construct
  REGION 1..131: note = Variant Type-1 fimbrial protein, A chain sequence
KATTVNGGTVHFKGEVVNAACAVDAGSVDQTVQLGQVRTASLAQEGATSSKVGFNIQLNDCDTNVASKAAVAFLGTKIDAGHTNVLALQSSAAGSDTNVGVQILDRTGAALTLDGATFSSETTLNNDTNTI

SEQ ID NO: 13 - protein, 18 aa, Escherichia coli
KLGYEVYEVTDPEELEVE

SEQ ID NO: 14 - protein, 18 aa, Escherichia coli
FKLGYEVYEVTDPEELEV

SEQ ID NO: 15 - protein, 9 aa, Escherichia coli
YEVTDPEEL

SEQ ID NO: 16 - protein, 15 aa, Escherichia coli
EGEFQLEPPLDTEEG

SEQ ID NO: 17 - protein, 15 aa, Escherichia coli
AQKYVLQDGDIMWQV

SEQ ID NO: 18 - protein, 15 aa, Escherichia coli
DEGEFQLEPPLDTEE

SEQ ID NO: 19 - protein, 15 aa, Escherichia coli
NAQKYVLQDGDIMWQ

SEQ ID NO: 20 - protein, 14 aa, Escherichia coli
EPPLDTEEGRAADE

SEQ ID NO: 21 - protein, 15 aa, Escherichia coli
NPGIDAEDANVQQFN

SEQ ID NO: 22 - protein, 14 aa, Escherichia coli
LEPPLDTEEGRAAD

SEQ ID NO: 23 - protein, 18 aa, Escherichia coli
FQLEPPLDTYVLQDGDIM

SEQ ID NO: 24 - protein, 9 aa, Escherichia coli
LDTEEGRAA

SEQ ID NO: 25 - protein, 9 aa, Escherichia coli
IDAEDANVQ

SEQ ID NO: 26 - protein, 6 aa, synthetic construct
  REGION 1..6: note = His tag
HHHHHH

SEQ ID NO: 27 - protein, 6 aa, synthetic construct
  REGION 1..6: note = Linker
GSGSGS

SEQ ID NO: 28 - protein, 10 aa, Escherichia coli
NNSLSQEVQN

SEQ ID NO: 29 - protein, 13 aa, Escherichia coli
NHLENNQSQSNGG

SEQ ID NO: 30 - protein, 18 aa, Escherichia coli
SALSLSSTAALAAATTVN

SEQ ID NO: 31 - protein, 8 aa, Escherichia coli
GATSSAVG

SEQ ID NO: 32 - protein, 4 aa, Escherichia coli
HHAH

SEQ ID NO: 33 - protein, 266 aa, synthetic construct
  REGION 1..266: note = CDP-RBD
HHHHHHDNNSLSQEVQNGSNHLENNQSQSNGGGSDSALSLSSKTAALAAATTVNDGSDGATSSAVGGSGSGSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 34 - protein, 540 aa, synthetic construct
  REGION 1..540: note = TRXtrunc- Vimentin protein sequence (540 aa)
MGKLTVAKLNIDQNPGTAPKYGIRGIPTLLLFKNGEVAATKVGALSKGQLKEFLDANLAGSGSGSGSSTRSVSSSSYRRMFGGSGTSSRPSSNRSYVTTSTRTYSLGSALRPSTSRSLYSSSPGGAYVTRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAESTLQSFRQDVDNASLARLDLERKVESLQEEIAFLKKLHDEEIQELQAQIQEQHVQIDVDVSKPDLTAALRDVRQQYESVAAKNLQEAEEWYKSKFADLSEAANRNNDALRQAKQESNEYRRQVQSLTCEVDALKGTNESLERQMREMEENFALEAANYQDTIGRLQDEIQNMKEEMARHLREYQDLLNVKMALDIEIATYRKLLEGEESRISLPLPTFSSLNLRETNLESLPLVDTHSKRTLLIKTVETRDGQVINETSQHHDDLERSHHHHHH

SEQ ID NO: 35 - protein, 542 aa, synthetic construct
  REGION 1..542: note = CDP-mVimentin protein (542 aa)
MDNNSLSQEVQNGSNHLENNQSQSNGGGSDSALSLSSKTAALAAATTVNDGSDGATSSAVGGSGSGSGSSTRSVSSSSYRRMFGGSGTSSRPSSNRSYVTTSTRTYSLGSALRPSTSRSLYSSSPGGAYVTRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAESTLQSFRQDVDNASLARLDLERKVESLQEEIAFLKKLHDEEIQELQAQIQEQHVQIDVDVSKPDLTAALRDVRQQYESVAAKNLQEAEEWYKSKFADLSEAANRNNDALRQAKQESNEYRRQVQSLTCEVDALKGTNESLERQMREMEENFALEAANYQDTIGRLQDEIQNMKEEMARHLREYQDLLNVKMALDIEIATYRKLLEGEESRISLPLPTFSSLNLRETNLESLPLVDTHSKRTLLIKTVETRDGQVINETSQHHDDLEEFHHHHHH

SEQ ID NO: 36 - protein, 550 aa, synthetic construct
  REGION 1..550: note = IDP1-mVimentin protein
MHHAHTMYATLEEAIDAAREEFLADNPGIDAEDANVQQFNAQKYVLQDGDIMWQVEFFADEGEEGEDDEDFVDEDDDGSGSGSGSSTRSVSSSSYRRMFGGSGTSSRPSSNRSYVTTSTRTYSLGSALRPSTSRSLYSSSPGGAYVTRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAESTLQSFRQDVDNASLARLDLERKVESLQEEIAFLKKLHDEEIQELQAQIQEQHVQIDVDVSKPDLTAALRDVRQQYESVAAKNLQEAEEWYKSKFADLSEAANRNNDALRQAKQESNEYRRQVQSLTCEVDALKGTNESLERQMREMEENFALEAANYQDTIGRLQDEIQNMKEEMARHLREYQDLLNVKMALDIEIATYRKLLEGEESRISLPLPTFSSLNLRETNLESLPLVDTHSKRTLLIKTVETRDGQVINETSQHHDDLE

SEQ ID NO: 37 - protein, 540 aa, synthetic construct
  REGION 1..540: note = IDP2-mVimentin protein
MHHAHNPGIDAEDANVQQFNAQKYVLQDGDIMWQVEGEFQLEPPLDTEEGRAAADEDDEDFVDEDDDGSGSGSGSSTRSVSSSSYRRMFGGSGTSSRPSSNRSYVTTSTRTYSLGSALRPSTSRSLYSSSPGGAYVTRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAESTLQSFRQDVDNASLARLDLERKVESLQEEIAFLKKLHDEEIQELQAQIQEQHVQIDVDVSKPDLTAALRDVRQQYESVAAKNLQEAEEWYKSKFADLSEAANRNNDALRQAKQESNEYRRQVQSLTCEVDALKGTNESLERQMREMEENFALEAANYQDTIGRLQDEIQNMKEEMARHLREYQDLLNVKMALDIEIATYRKLLEGEESRISLPLPTFSSLNLRETNLESLPLVDTHSKRTLLIKTVETRDGQVINETSQHHDDLE

SEQ ID NO: 38 - protein, 519 aa, synthetic construct
  REGION 1..519: note = IDP3-mVimentin protein
MHHAHNPGIDAEDANVQQFNAQKYVLQDGDIMWQVDDEDFVDEDDDGSGSGSGSSTRSVSSSSYRRMFGGSGTSSRPSSNRSYVTTSTRTYSLGSALRPSTSRSLYSSSPGGAYVTRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAESTLQSFRQDVDNASLARLDLERKVESLQEEIAFLKKLHDEEIQELQAQIQEQHVQIDVDVSKPDLTAALRDVRQQYESVAAKNLQEAEEWYKSKFADLSEAANRNNDALRQAKQESNEYRRQVQSLTCEVDALKGTNESLERQMREMEENFALEAANYQDTIGRLQDEIQNMKEEMARHLREYQDLLNVKMALDIEIATYRKLLEGEESRISLPLPTFSSLNLRETNLESLPLVDTHSKRTLLIKTVETRDGQVINETSQHHDDLE

SEQ ID NO: 39 - protein, 282 aa, synthetic construct
  REGION 1..282: note = SARS-CoV-2 sequence used in CDP-complete
SNNLDSKVGGNYNYLYRLFRKSNLKPFERDISTEIYQAGSTPCNGVEGFNCYFPLQSYGFQPTNGVGYQGSGSGSPKKSTNLVKNKCVNFNFNGLTGTGVLTESNKKFLPFGSGSGSTEVPVAIHADQLTPTWRVYSTGSNVFQTRGSGSGSYQTQTNSPRRARSVASQSIIAYTGSGSGSKQIYKTPPIKDFGGFNFSQILPDPSKPSKRSFIEDLLFNKVTLADAGFIKQYGDCLGDIAARDGSGSGSTQNVLYENQKLIANQFNSAIGKIQDSLSSTAS

SEQ ID NO: 40 - protein, 146 aa, synthetic construct
  REGION 1..146: note = The SARS-CoV-2 sequence used in CDP-binding
SNNLDSKVGGNYNYLYRLFRKSNLKPFERDISTEIYQAGSTPCNGVEGFNCYFPLQSYGFQPTNGVGYQGSGSGSPKKSTNLVKNKCVNFNFNGLTGTGVLTESNKKFLPFGSGSGSTEVPVAIHADQLTPTWRVYSTGSNVFQTR

SEQ ID NO: 41 - protein, 130 aa, synthetic construct
  REGION 1..130: note = The SARS-CoV-2 sequence used in CDP-fusion
YQTQTNSPRRARSVASQSIIAYTGSGSGSKQIYKTPPIKDFGGFNFSQILPDPSKPSKRSFIEDLLFNKVTLADAGFIKQYGDCLGDIAARDGSGSGSTQNVLYENQKLIANQFNSAIGKIQDSLSSTAS

SEQ ID NO: 42 - protein, 208 aa, Influenza virus
CNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAG

SEQ ID NO: 43 - protein, 214 aa, Influenza virus
WLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYS

SEQ ID NO: 44 - protein, 430 aa, Influenza virus
CNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGGSGSGSELWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYS

SEQ ID NO: 45 - protein, 284 aa, synthetic construct
  REGION 1..284: note = CDP - H1
HMHHHHHHDNNSLSQEVQNGSNHLENNQSQSNGGGSDSALSLSSKTAALAAATTVNDGSDGATSSAVGGSGSGSGSCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAG

SEQ ID NO: 46 - protein, 290 aa, synthetic construct
  REGION 1..290: note = CDP - N1
HMHHHHHHDNNSLSQEVQNGSNHLENNQSQSNGGGSDSALSLSSKTAALAAATTVNDGSDGATSSAVGGSGSGSGSWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYS

SEQ ID NO: 47 - protein, 506 aa, synthetic construct
  REGION 1..506: note = CDP - H1N1
HMHHHHHHDNNSLSQEVQNGSNHLENNQSQSNGGGSDSALSLSSKTAALAAATTVNDGSDGATSSAVGGSGSGSGSCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGGSGSGSELWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYS

SEQ ID NO: 48 - protein, 149 aa, synthetic construct
  REGION 1..149: note = His-TRXtr-mApelin protein
MHHHHHHGKLTVAKLNIDQNPGTAPKYGIRGIPTLLLFKNGEVAATKVGALSKGQLKEFLDANLAGSGSGSGSNLRLCVQALLLLWLSLTAVCGVPLMLPPDGTGLEEGSMRYLVKPRTSRTGPGAWQGGRRKFRRQRPRLSHKGPMPF

SEQ ID NO: 49 - protein, 151 aa, synthetic construct
  REGION 1..151: note = His-CDP-mApelin protein
MHHHHHHDNNSLSQEVQNGSNHLENNQSQSNGGGSDSALSLSSKTAALAAATTVNDGSDGATSSAVGGSGSGSGSNLRLCVQALLLLWLSLTAVCGVPLMLPPDGTGLEEGSMRYLVKPRTSRTGPGAWQGGRRKFRRQRPRLSHKGPMPF

SEQ ID NO: 50 - protein, 161 aa, synthetic construct
  REGION 1..161: note = CDP2.1-Apelin
MHHAHTMYATLEEAIDAAREEFLADNPGIDAEDANVQQFNAQKYVLQDGDIMWQVEFFADEGEEGEDDEDFVDEDDDGSGSGSGSNLRLCVQALLLLWLSLTAVCGVPLMLPPDGTGLEEGSMRYLVKPRTSRTGPGAWQGGRRKFRRQRPRLSHKGPMPF

SEQ ID NO: 51 - protein, 151 aa, synthetic construct
  REGION 1..151: note = CDP2.2-Apelin
MHHAHNPGIDAEDANVQQFNAQKYVLQDGDIMWQVEGEFQLEPPLDTEEGRAAADEDDEDFVDEDDDGSGSGSGSNLRLCVQALLLLWLSLTAVCGVPLMLPPDGTGLEEGSMRYLVKPRTSRTGPGAWQGGRRKFRRQRPRLSHKGPMPF

SEQ ID NO: 52 - protein, 130 aa, synthetic construct
  REGION 1..130: note = CDP2.3-Apelin
MHHAHNPGIDAEDANVQQFNAQKYVLQDGDIMWQVDDEDFVDEDDDGSGSGSGSNLRLCVQALLLLWLSLTAVCGVPLMLPPDGTGLEEGSMRYLVKPRTSRTGPGAWQGGRRKFRRQRPRLSHKGPMPF

SEQ ID NO: 53 - protein, 558 aa, synthetic construct
  REGION 1..558: note = TRXtr-Notum
HMHHHHHHGKLTVAKLNIDQNPGTAPKYGIRGIPTLLLFKNGEVAATKVGALSKGQLKEFLDANLAGSGSGSGSSEGRKTWRRRGQQPPQPPPPPPLPQRAEVEPGAGQPVESFPLDFTAVEGNMDSFMAQVKSLAQSLYPCSAQQLNEDLRLHLLLNTSVTCNDGSPAGYYLKESKGSRRWLLFLEGGWYCFNRENCDSRYSTMRRLMSSKDWPHTRTGTGILSSQPEENPHWWNANMVFIPYCSSDVWSGASPKSDKNEYAFMGSLIIQEVVRELLGKGLSGAKVLLLAGSAAGGTGVLLNVDRVAELLEELGYPSIQVRGLADSGWFLDNKQYRRSDCIDTINCAPTDAIRRGIRYWSGMVPERCQRQFKEGEEWNCFFGYKVYPTLRCPVFVVQWLFDEAQLTVDNVHLTGQPVQEGQWLYIQNLGRELRGTLKDVQASFAPACLSHEIIIRSYWTDVQVKGTSLPRALHCWDRSFHDSHKASKTPMKGCPFHLVDSCPWPHCNPSCPTIRDQFTGQEMNVAQFLMHMGFDVQTVAQQQGMEPSKLLGMLSNGN

SEQ ID NO: 54 - protein, 560 aa, synthetic construct
  REGION 1..560: note = CDP - mNotum
HMHHHHHHDNNSLSQEVQNGSNHLENNQSQSNGGGSDSALSLSSKTAALAAATTVNDGSDGATSSAVGGSGSGSGSSEGRKTWRRRGQQPPQPPPPPPLPQRAEVEPGAGQPVESFPLDFTAVEGNMDSFMAQVKSLAQSLYPCSAQQLNEDLRLHLLLNTSVTCNDGSPAGYYLKESKGSRRWLLFLEGGWYCFNRENCDSRYSTMRRLMSSKDWPHTRTGTGILSSQPEENPHWWNANMVFIPYCSSDVWSGASPKSDKNEYAFMGSLIIQEVVRELLGKGLSGAKVLLLAGSAAGGTGVLLNVDRVAELLEELGYPSIQVRGLADSGWFLDNKQYRRSDCIDTINCAPTDAIRRGIRYWSGMVPERCQRQFKEGEEWNCFFGYKVYPTLRCPVFVVQWLFDEAQLTVDNVHLTGQPVQEGQWLYIQNLGRELRGTLKDVQASFAPACLSHEIIIRSYWTDVQVKGTSLPRALHCWDRSFHDSHKASKTPMKGCPFHLVDSCPWPHCNPSCPTIRDQFTGQEMNVAQFLMHMGFDVQTVAQQQGMEPSKLLGMLSNGN

SEQ ID NO: 55 - protein, 570 aa, synthetic construct
  REGION 1..570: note = CDP2.1-mNotum
HMHHAHTMYATLEEAIDAAREEFLADNPGIDAEDANVQQFNAQKYVLQDGDIMWQVEFFADEGEEGEDDEDFVDEDDDGSGSGSGSSEGRKTWRRRGQQPPQPPPPPPLPQRAEVEPGAGQPVESFPLDFTAVEGNMDSFMAQVKSLAQSLYPCSAQQLNEDLRLHLLLNTSVTCNDGSPAGYYLKESKGSRRWLLFLEGGWYCFNRENCDSRYSTMRRLMSSKDWPHTRTGTGILSSQPEENPHWWNANMVFIPYCSSDVWSGASPKSDKNEYAFMGSLIIQEVVRELLGKGLSGAKVLLLAGSAAGGTGVLLNVDRVAELLEELGYPSIQVRGLADSGWFLDNKQYRRSDCIDTINCAPTDAIRRGIRYWSGMVPERCQRQFKEGEEWNCFFGYKVYPTLRCPVFVVQWLFDEAQLTVDNVHLTGQPVQEGQWLYIQNLGRELRGTLKDVQASFAPACLSHEIIIRSYWTDVQVKGTSLPRALHCWDRSFHDSHKASKTPMKGCPFHLVDSCPWPHCNPSCPTIRDQFTGQEMNVAQFLMHMGFDVQTVAQQQGMEPSKLLGMLSNGN

SEQ ID NO: 56 - protein, 560 aa, synthetic construct
  REGION 1..560: note = CDP2.2- mNotum
HMHHAHNPGIDAEDANVQQFNAQKYVLQDGDIMWQVEGEFQLEPPLDTEEGRAAADEDDEDFVDEDDDGSGSGSGSSEGRKTWRRRGQQPPQPPPPPPLPQRAEVEPGAGQPVESFPLDFTAVEGNMDSFMAQVKSLAQSLYPCSAQQLNEDLRLHLLLNTSVTCNDGSPAGYYLKESKGSRRWLLFLEGGWYCFNRENCDSRYSTMRRLMSSKDWPHTRTGTGILSSQPEENPHWWNANMVFIPYCSSDVWSGASPKSDKNEYAFMGSLIIQEVVRELLGKGLSGAKVLLLAGSAAGGTGVLLNVDRVAELLEELGYPSIQVRGLADSGWFLDNKQYRRSDCIDTINCAPTDAIRRGIRYWSGMVPERCQRQFKEGEEWNCFFGYKVYPTLRCPVFVVQWLFDEAQLTVDNVHLTGQPVQEGQWLYIQNLGRELRGTLKDVQASFAPACLSHEIIIRSYWTDVQVKGTSLPRALHCWDRSFHDSHKASKTPMKGCPFHLVDSCPWPHCNPSCPTIRDQFTGQEMNVAQFLMHMGFDVQTVAQQQGMEPSKLLGMLSNGN

SEQ ID NO: 57 - protein, 539 aa, synthetic construct
  REGION 1..539: note = CDP2.3- mNotum
HMHHAHNPGIDAEDANVQQFNAQKYVLQDGDIMWQVDDEDFVDEDDDGSGSGSGSSEGRKTWRRRGQQPPQPPPPPPLPQRAEVEPGAGQPVESFPLDFTAVEGNMDSFMAQVKSLAQSLYPCSAQQLNEDLRLHLLLNTSVTCNDGSPAGYYLKESKGSRRWLLFLEGGWYCFNRENCDSRYSTMRRLMSSKDWPHTRTGTGILSSQPEENPHWWNANMVFIPYCSSDVWSGASPKSDKNEYAFMGSLIIQEVVRELLGKGLSGAKVLLLAGSAAGGTGVLLNVDRVAELLEELGYPSIQVRGLADSGWFLDNKQYRRSDCIDTINCAPTDAIRRGIRYWSGMVPERCQRQFKEGEEWNCFFGYKVYPTLRCPVFVVQWLFDEAQLTVDNVHLTGQPVQEGQWLYIQNLGRELRGTLKDVQASFAPACLSHEIIIRSYWTDVQVKGTSLPRALHCWDRSFHDSHKASKTPMKGCPFHLVDSCPWPHCNPSCPTIRDQFTGQEMNVAQFLMHMGFDVQTVAQQQGMEPSKLLGMLSNGN

SEQ ID NO: 58 - protein, 255 aa, synthetic construct
  REGION 1..255: note = TRXtr-Timp1
HMHHHHHHGKLTVAKLNIDQNPGTAPKYGIRGIPTLLLFKNGEVAATKVGALSKGQLKEFLDANLAGSGSGSGSCSCAPPHPQTAFCNSDLVIRAKFMGSPEINETTLYQRYKIKMTKMLKGFKAVGNAADIRYAYTPVMESLCGYAHKSQNRSEEFLITGRLRNGNLHISACSFLVPWRTLSPAQQRAFSKTYSAGCGVCTVFPCLSIPCKLESDTHCLWTDQVLVGSEDYQSRHFACLPRNPGLCTWRSLGAR

SEQ ID NO: 59 - protein, 257 aa, synthetic construct
  REGION 1..257: note = CDP-Timp1
HMHHHHHHDNNSLSQEVQNGSNHLENNQSQSNGGGSDSALSLSSKTAALAAATTVNDGSDGATSSAVGGSGSGSGSCSCAPPHPQTAFCNSDLVIRAKFMGSPEINETTLYQRYKIKMTKMLKGFKAVGNAADIRYAYTPVMESLCGYAHKSQNRSEEFLITGRLRNGNLHISACSFLVPWRTLSPAQQRAFSKTYSAGCGVCTVFPCLSIPCKLESDTHCLWTDQVLVGSEDYQSRHFACLPRNPGLCTWRSLGAR

SEQ ID NO: 60 - protein, 267 aa, synthetic construct
  REGION 1..267: note = CDP2.1-Timp1
HMHHAHTMYATLEEAIDAAREEFLADNPGIDAEDANVQQFNAQKYVLQDGDIMWQVEFFADEGEEGEDDEDFVDEDDDGSGSGSGSCSCAPPHPQTAFCNSDLVIRAKFMGSPEINETTLYQRYKIKMTKMLKGFKAVGNAADIRYAYTPVMESLCGYAHKSQNRSEEFLITGRLRNGNLHISACSFLVPWRTLSPAQQRAFSKTYSAGCGVCTVFPCLSIPCKLESDTHCLWTDQVLVGSEDYQSRHFACLPRNPGLCTWRSLGAR

SEQ ID NO: 61 - protein, 257 aa, synthetic construct
  REGION 1..257: note = CDP2.2-Timp1
HMHHAHNPGIDAEDANVQQFNAQKYVLQDGDIMWQVEGEFQLEPPLDTEEGRAAADEDDEDFVDEDDDGSGSGSGSCSCAPPHPQTAFCNSDLVIRAKFMGSPEINETTLYQRYKIKMTKMLKGFKAVGNAADIRYAYTPVMESLCGYAHKSQNRSEEFLITGRLRNGNLHISACSFLVPWRTLSPAQQRAFSKTYSAGCGVCTVFPCLSIPCKLESDTHCLWTDQVLVGSEDYQSRHFACLPRNPGLCTWRSLGAR

SEQ ID NO: 62 - protein, 236 aa, synthetic construct
  REGION 1..236: note = CDP2.3-Timp1
HMHHAHNPGIDAEDANVQQFNAQKYVLQDGDIMWQVDDEDFVDEDDDGSGSGSGSCSCAPPHPQTAFCNSDLVIRAKFMGSPEINETTLYQRYKIKMTKMLKGFKAVGNAADIRYAYTPVMESLCGYAHKSQNRSEEFLITGRLRNGNLHISACSFLVPWRTLSPAQQRAFSKTYSAGCGVCTVFPCLSIPCKLESDTHCLWTDQVLVGSEDYQSRHFACLPRNPGLCTWRSLGAR

SEQ ID NO: 63 - protein, 357 aa, synthetic construct
  REGION 1..357: note = CDP-complete
MHHHHHHDNNSLSQEVQNGSNHLENNQSQSNGGGSDSALSLSSKTAALAAATTVNDGSDGATSSAVGGSGSGSGSSNNLDSKVGGNYNYLYRLFRKSNLKPFERDISTEIYQAGSTPCNGVEGFNCYFPLQSYGFQPTNGVGYQGSGSGSPKKSTNLVKNKCVNFNFNGLTGTGVLTESNKKFLPFGSGSGSTEVPVAIHADQLTPTWRVYSTGSNVFQTRGSGSGSYQTQTNSPRRARSVASQSIIAYTGSGSGSKQIYKTPPIKDFGGFNFSQILPDPSKPSKRSFIEDLLFNKVTLADAGFIKQYGDCLGDIAARDGSGSGSTQNVLYENQKLIANQFNSAIGKIQDSLSSTAS

SEQ ID NO: 64 - protein, 221 aa, synthetic construct
  REGION 1..221: note = CDP-binding
MHHHHHHDNNSLSQEVQNGSNHLENNQSQSNGGGSDSALSLSSKTAALAAATTVNDGSDGATSSAVGGSGSGSGSSNNLDSKVGGNYNYLYRLFRKSNLKPFERDISTEIYQAGSTPCNGVEGFNCYFPLQSYGFQPTNGVGYQGSGSGSPKKSTNLVKNKCVNFNFNGLTGTGVLTESNKKFLPFGSGSGSTEVPVAIHADQLTPTWRVYSTGSNVFQTR

SEQ ID NO: 65 - protein, 205 aa, synthetic construct
  REGION 1..205: note = CDP-fusion
MHHHHHHDNNSLSQEVQNGSNHLENNQSQSNGGGSDSALSLSSKTAALAAATTVNDGSDGATSSAVGGSGSGSGSYQTQTNSPRRARSVASQSIIAYTGSGSGSKQIYKTPPIKDFGGFNFSQILPDPSKPSKRSFIEDLLFNKVTLADAGFIKQYGDCLGDIAARDGSGSGSTQNVLYENQKLIANQFNSAIGKIQDSLSSTAS

SEQ ID NO: 66 - DNA, 1089 bp, synthetic construct
  source 1..1089: mol_type = other DNA
  misc_feature 1..1089: note = CDP-complete
catatgcaccatcaccatcaccatgataacaactcactgtcgcaggaagttcaaaatggttctaaccacttagaaaacaaccagagccagagtaatggcggcggttctgattcggctctgtccctcagttctaaaacagcggctctggccgctgccacgacggttaatgatggttctgatggagcaaccagttctgctgtcggtggttctggttctggtagcggatccagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagggttctggttctggtagcccgaaaaaaagcaccaacctggtgaaaaacaaatgcgtgaactttaactttaacggcctgaccggcaccggcgtgctgaccgaaagcaacaaaaaatttctgccgtttggttctggttctggtagcaccgaagtgccggtggcgattcatgcggatcagctgaccccgacctggcgcgtgtatagcaccggcagcaacgtgtttcagacccgcggttctggttctggtagctatcagacccagaccaacagcccgcgccgcgcgcgcagcgtggcgagccagagcattattgcgtataccggttctggttctggtagcaaacagatttataaaaccccgccgattaaagattttggcggctttaactttagccagattctgccggatccgagcaaaccgagcaaacgcagctttattgaagatctgctgtttaacaaagtgaccctggcggatgcgggctttattaaacagtatggcgattgcctgggcgatattgcggcgcgcgatggttctggttctggtagcacccagaacgtgctgtatgaaaaccagaaactgattgcgaaccagtttaacagcgcgattggcaaaattcaggatagcctgagcagcaccgcgagctagtgataactcgag

SEQ ID NO: 67 - DNA, 681 bp, synthetic construct
  source 1..681: mol_type = other DNA
  misc_feature 1..681: note = CDP-binding
catatgcaccatcaccatcaccatgataacaactcactgtcgcaggaagttcaaaatggttctaaccacttagaaaacaaccagagccagagtaatggcggcggttctgattcggctctgtccctcagttctaaaacagcggctctggccgctgccacgacggttaatgatggttctgatggagcaaccagttctgctgtcggtggttctggttctggtagcggatccagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagggttctggttctggtagcccgaaaaaaagcaccaacctggtgaaaaacaaatgcgtgaactttaactttaacggcctgaccggcaccggcgtgctgaccgaaagcaacaaaaaatttctgccgtttggttctggttctggtagcaccgaagtgccggtggcgattcatgcggatcagctgaccccgacctggcgcgtgtatagcaccggcagcaacgtgtttcagacccgctagtgataactcgag

SEQ ID NO: 68 - DNA, 633 bp, synthetic construct
  source 1..633: mol_type = other DNA
  misc_feature 1..633: note = CDP-fusion
catatgcaccatcaccatcaccatgataacaactcactgtcgcaggaagttcaaaatggttctaaccacttagaaaacaaccagagccagagtaatggcggcggttctgattcggctctgtccctcagttctaaaacagcggctctggccgctgccacgacggttaatgatggttctgatggagcaaccagttctgctgtcggtggttctggttctggtagcggatcctatcagacccagaccaacagcccgcgccgcgcgcgcagcgtggcgagccagagcattattgcgtataccggttctggttctggtagcaaacagatttataaaaccccgccgattaaagattttggcggctttaactttagccagattctgccggatccgagcaaaccgagcaaacgcagctttattgaagatctgctgtttaacaaagtgaccctggcggatgcgggctttattaaacagtatggcgattgcctgggcgatattgcggcgcgcgatggttctggttctggtagcacccagaacgtgctgtatgaaaaccagaaactgattgcgaaccagtttaacagcgcgattggcaaaattcaggatagcctgagcagcaccgcgagctagtgataactcgag

SEQ ID NO: 69 - protein, 69 aa, SARS-CoV-2
SNNLDSKVGGNYNYLYRLFRKSNLKPFERDISTEIYQAGSTPCNGVEGFNCYFPLQSYGFQPTNGVGYQ

SEQ ID NO: 70 - protein, 36 aa, SARS-CoV-2
PKKSTNLVKNKCVNFNFNGLTGTGVLTESNKKFLPF

SEQ ID NO: 71 - protein, 29 aa, SARS-CoV-2
TEVPVAIHADQLTPTWRVYSTGSNVFQTR

SEQ ID NO: 72 - protein, 23 aa, SARS-CoV-2
YQTQTNSPRRARSVASQSIIAYT

SEQ ID NO: 73 - protein, 63 aa, SARS-CoV-2
KQIYKTPPIKDFGGFNFSQILPDPSKPSKRSFIEDLLFNKVTLADAGFIKQYGDCLGDIAARD

SEQ ID NO: 74 - protein, 32 aa, SARS-CoV-2
TQNVLYENQKLIANQFNSAIGKIQDSLSSTAS

SEQ ID NO: 75 - protein, 4 aa, synthetic construct
  REGION 1..4: note = Linker
GSGS